(19) United States
(12) Patent Application Publication
(10) Pub. No.: US 2012/0039806 A1
(43) Pub. Date: Feb. 16, 2012

(54) Compounds and Methods for Modulating an Immune Response

(76) Inventors: Mireille Hanna Lahoud, Victoria (AU); Irina Caminschi, Victoria (AU); Jian-Guo Zhang, Victoria (AU); Mark Francis Van Delft, Victoria (AU); David Ching Siang Huang, Victoria (AU); Nicos Anthony NIcola, Victoria (AU); Alan Cowman, Victoria (AU); Mark Dexter Wright, Victoria (AU)

(21) Appl. No.: 13/203,682

(22) PCT Filed: Mar. 22, 2010
(86) PCT No.: PCT/AU10/00325

§ 371 Date: Nov. 1, 2011

Related U.S. Application Data
(60) Provisional application No. 61/162,616, filed on Mar. 23, 2009.

(51) Int. Cl. A61K 49/00; A61K 9/48; A61K 39/00; A61K 47/00; G01N 33/567; C12N 5/10; C12N 1/21; C12N 1/19; C12N 15/63; A61P 37/00; A61P 35/00; A61K 38/17; A61K 31/7088; C07K 14/00; C07K 16/00; A61P 37/08; A61P 31/00; A61P 33/02; A61P 29/00; A61K 9/127

(52) U.S. Cl. 424/9.1; 424/450; 424/451; 424/184.1; 424/185.1; 424/278.1; 435/7.2; 435/325; 435/348; 435/375; 435/419; 435/252.3; 435/254.2; 435/320.1; 514/4.4; 514/19.3; 514/21.2; 514/44 R; 514/44 A; 530/350; 530/387.3; 530/387.9; 530/391.3; 530/391.7; 536/23.4

(57) ABSTRACT

The present invention relates to the identification of proteins which bind the dendritic cell marker known as Clec9A. The present invention provides new compounds for targeting therapeutic agents such as antigens to dendritic cells. Also provided are methods of modulating a humoral and/or T cell mediated immune response to the antigen, methods of delivery of a cytotoxic agent to dendritic cells thereof involved in diseased states, methods of modulating the uptake and/or clearance of cells with a disrupted cell membrane, cells infected with a pathogen, dying cells or dead cells, or a portion thereof, and methods of modulating antigen recognition, processing and/or presentation, as well as immune responses to material derived from cells with a disrupted cell membrane, cells infected with a pathogen, dying cells or dead cells, or a portion thereof.

### FIELD OF THE INVENTION

The present invention relates to the identification of proteins which bind the dendritic cell marker known as Clec9A. The present invention provides new compounds for targeting therapeutic agents such as antigens to dendritic cells. Also provided are methods of modulating a humoral and/or T cell mediated immune response to the antigen, methods of delivery of a cytotoxic agent to dendritic cells thereof involved in diseased states, methods of modulating the uptake and/or clearance of cells with a disrupted cell membrane, cells infected with a pathogen, dying cells or dead cells, or a portion thereof, and methods of modulating antigen recognition, processing and/or presentation, as well as immune responses to material derived from cells with a disrupted cell membrane, cells infected with a pathogen, dying cells or dead cells, or a portion thereof.

### BACKGROUND OF THE INVENTION

### Dendritic Cell Biology

Dendritic cells (DC) are found in most tissues, where they continuously sample the antigenic environment and use several types of receptors to monitor for invading pathogens. In steady state, and at an increased rate upon detection of pathogens, sentinel DC in non-lymphoid tissues migrate to the lymphoid organs where they present to T cells the Ag they have collected and processed. The phenotype acquired by the T cell depends on the context in which the DC presents its Ag. If the Ag is an innocuous self-component, DC may induce various forms of T cell unresponsiveness (tolerance) (Kenna et al., 2008). However, if the Ag is derived from a pathogen, or damaged self, DC receive danger signals, become activated and the T cells are then stimulated to become effectors, necessary to provide protective immunity (Heath and Villadangos, 2005). Moreover, DC can instruct effector T cells to acquire distinct abilities (migratory properties, cytokine secretion) tailored to fighting particular pathogens. The information required to tailor immunity is determined by the DC-pathogen interaction and is communicated by the DC to T cells via secreted cytokines or membrane proteins.

### DC Heterogeneity

The DC network is composed of distinct DC subtypes (Shortman and Naik, 2007). DC can be broadly classified into plasmacytoid pre-DC (pDC) and conventional DC (cDC). pDC secrete high levels of IFNα upon stimulation, and only develop DC form after activation (Hochrein et al., 2001; O'Keeffe et al., 2002). cDC have immediate DC form and Ag presentation function, and may be divided into “lymphoid tissue resident” DC and classical “migratory” DC which arrive in lymph nodes (LN) via the lymph. Lymphoid tissue resident DC in mice may in turn be divided into the CD8⁺ and CD8⁻ subsets (Belz et al., 2004; Lahoud et al., 2006; Henri et al., 2001). In addition, inflammatory DC develop from monocytes as a consequence of infection or inflammation (Shortman and Naik, 2007). Importantly, DC subtypes share many functions (uptake, processing and presentation of Ag to activate naïve T cells), but also exhibit subset-specific roles. These include differential expression of Toll-like receptors, production of particular chemokines, IL-12 secretion, restricted primarily to CD8⁺DC which directs T cells to a Th1 cytokine profile and cross-presentation of exogenous Ag via MHC I, mainly restricted to CD8⁺DC, which allows these DC to be major presenters of viral Ag to the CD8⁺ T cells, allowing them to develop cytotoxic function.

Humans most likely contain equivalents of these DC subsets, as mouse and human DC extracted from the same tissue (blood or thymus) are similar (Vandenabeele et al., 2001; O'Keeffe et al., 2003). However, human equivalents of most murine lymphoid organ resident DC subsets remain unknown, due to differences in markers between species (CD8α is not on human DC) and the difficulty of obtaining human lymphoid organs for detailed analyses. Definition of DC subset-specific markers conserved between mice and humans is therefore a major challenge.

### Cell Death and the Immune Response

Apoptosis occurs continuously during development and within the immune system. It is characterised by convoluting of cell membranes, condensing of nuclei and fragmenting of cells into apoptotic bodies that still retain their cellular contents; these are usually engulfed by “phagocytes” without release of cellular contents. In contrast, necrosis, which may occur during injury or infection, is characterised by cell membrane rupturing and cellular content release. However, cellular contents may also be released from apoptotic cells if they are not rapidly engulfed (secondary necrosis). Rapid clearance of apoptotic cells generally promotes an immunosuppressive environment that avoids inflammatory responses to self-Ag, whereas necrosis or failure of apoptotic cell clearance promotes immune responses to self-Ag (Hume, 2008; Nagata, 2007; Peng et al., 2007). Thus, early recognition of apoptotic cells is essential for homeostatic maintenance and prevention of autoimmunity.

### Recognition of Dead/Dying Cells

Phagocytes, including DC, sense many molecular changes in dying cells. Molecules normally within the cell may be secreted or presented on the cell surface of apoptotic cells, or finally exposed when the cell membrane is disrupted. Existing molecules may be modified or new molecules produced in response to stress. An example of such a molecule is phosphotidylserine (PS) which is a lipid that is translocated from the inside to the outside of the cell membrane early in the apoptotic process, and serves as an “eat me” signal. Many molecules on phagocytes mediate the recognition of PS including CD36, MFG-E8 and Tim4.

Another example of molecules which are produced in response to stress are heat shock or stress proteins (Hsp), which serve as molecular chaperones in intact cells (Binder et al., 2004), and may provide signals to DC from stressed/dying cells (Delneste, 2004). Proposed Hsp receptors on phagocytes and DC include CD91, Lox1/Clec8, CD40, TLR2, TLR4, CD36. Furthermore, immunisation with Hsp-chaperoned peptides is extremely effective, requiring only pg amounts of protein.

### Recognition and Uptake of Dead Cells by DC

Both macrophages and DC can take up dead or dying cells. Many of the scavenger receptors on macrophages are also found on DC. However, only DC have the capacity to process cell components and then effectively present them to, and activate, naive T cells. While the uptake of apoptotic cells by DC in the absence of additional pathogen signals generally induces tolerance (Steinman et al., 2000), uptake of necrotic cells induces DC maturation and stimulation of immune response (Sauter et al., 2000). Thus differential recognition of these states by receptors on DC is crucial to the immune system. Murine CD8⁺ cDC are more efficient than CD8⁻ DC at both uptake of apoptotic/dead cells and subsequent presentation of cell bound and viral Ag to T cells, particularly the “cross-presentation” on MHC I to CD8 T cells (Belz et al., 2004; Schnorrer et al., 2006; Belz et al., 2005; Iyoda et al., 2002). Human pDC have been claimed to be effective at uptake of dying cells (Hoeffel et al., 2007). Thus, the selective expression of dead cell uptake receptors by DC subtypes is an important issue.

### Clec9A

Clec9A (also referred to by the present inventors as 5B6) is one of a family of C-type lectin-like molecules. In humans, Clec9A expression is restricted to a subset of dendritic cells which appear to be the human equivalent of mouse CD8+ dendritic cells. Clec9A can be targeted to modulate the immune response (Caminschi et al., 2008). To date, antibodies which bind Clec9A, as well as soluble forms of Clec9A, have been determined to be useful for targeting antigens to dendritic cells. However, natural ligands of Clec9A had not been identified before now.

### SUMMARY OF THE INVENTION

The present inventors have identified ligands of Clec9A. These ligands can be used to target therapeutic agents to Clec9A expressing cells such as dendritic cells.

In a first aspect, the present invention provides a compound comprising a polypeptide conjugated to a therapeutic agent, wherein the polypeptide comprises

i) an amino acid sequence as provided in any one of SEQ ID NO's 48 to 80;

ii) an amino acid sequence which is at least 50% identical to any one or more of SEQ ID NO's 48 to 80; and/or

iii) a biologically active fragment of i) or ii), and wherein the polypeptide of the compound binds a second polypeptide comprising

a) an amino acid sequence as provided in any one of SEQ ID NO's 1 to 8; and/or

b) an amino acid sequence which is at least 50% identical to any one or more of SEQ ID NO's 1 to 8.

Examples of suitable therapeutic agents include, but are not limited to, an antigen, a cytotoxic agent, a drug and/or pharmacological agent.

The antigen can be any molecule that induces an immune response in an animal. Examples include, but are not limited to, a cancer antigen, a self antigen, an allergen, and/or an antigen from a pathogenic and/or infectious organism.

In an embodiment, the antigen from a pathogenic and/or infectious organism can be from, but not limited to, Plasmodium falciparum or Plasmodium vivax.

In another aspect, the present invention provides a compound comprising a polypeptide conjugated to a detectable label, wherein the polypeptide comprises

i) an amino acid sequence as provided in any one of SEQ ID NO's 48 to 80;

ii) an amino acid sequence which is at least 50% identical to any one or more of SEQ ID NO's 48 to 80; and/or

iii) a biologically active fragment of i) or ii), and wherein the polypeptide of the compound binds a second polypeptide comprising

a) an amino acid sequence as provided in any one of SEQ ID NO's 1 to 8; and/or

b) an amino acid sequence which is at least 50% identical to any one or more of SEQ ID NO's 1 to 8.

In another aspect, the present invention provides a compound that binds a polypeptide which comprises:

i) an amino acid sequence as provided in any one of SEQ ID NO's 48 to 80;

ii) an amino acid sequence which is at least 50% identical to any one or more of SEQ ID NO's 48 to 80; and/or

iii) a biologically active fragment of i) or ii).

In a preferred embodiment of the above aspect, the compound is not an antibody which binds Clec9A, Clec9A per se or a fragment of Clec9A which binds

Clec9A such as a soluble fragment.

In a preferred embodiment, the compound is a polypeptide.

In another preferred embodiment of the above aspect, the compound is an antibody or antigenic binding fragment thereof. Examples of antibodies or antigenic binding fragment thereof include, but are not limited to, a monoclonal antibody, humanized antibody, single chain antibody, diabody, triabody, or tetrabody.

In a further preferred embodiment, the compound of the above aspect is conjugated to a therapeutic agent. Examples of such therapeutic agents are described above in relation to the first aspect.

In a further preferred embodiment, the compound of the above aspect is detectably labelled.

In a further aspect, the present invention provides a composition comprising a compound of the invention and a pharmaceutically acceptable carrier:

In an embodiment, the composition further comprises an adjuvant.

In another embodiment, the compound is encapsulated in, or exposed on the surface of, a liposome.

In a further aspect, the present invention provides a method of modulating an immune response in a subject, the method comprising administering to the subject a compound of the invention and/or a composition of the invention.

In an embodiment, the immune response to an antigen is induced and/or enhanced.

In a particularly preferred embodiment, the immune response is modulated by enhancing a helper T cell response.

In a further preferred embodiment, the immune response is modulated by the activation of CD4+ and/or CD8+ T cells.

In another particularly preferred embodiment, the immune response is modulated by enhancing B cell antibody production. Examples of antibodies produced include, but are not necessarily limited to, IgG1, IgG2b, IgG2c, IgG3, IgG4, IgM, IgA1, IgA2, IgE and/or IgD antibody isotypes.

In a further preferred embodiment, the immune response is modulated by generating a memory response.

In a particularly preferred embodiment, the subject is administered with a compound comprising the antigen.

In another embodiment, an immune response to a self antigen or allergen is reduced. In this embodiment, it is preferred that the immune response is modulated by suppressing a T cell response and/or a B cell antibody response.

In another aspect, the present invention provides a method of modulating an immune response to an antigen in a subject, the method comprising exposing dendritic cells or precursors thereof in vitro to a compound of the invention, and/or a composition of the invention, and administering said cells to the subject.

In an embodiment, the cells have been isolated from the subject.

Preferably, a humoral and/or T cell mediated response is modulated.

In a further embodiment, naïve CD8+ T cell activation, and/or naïve CD4+ T cell activation, is modulated.

In yet another embodiment, the humoral response comprises the production of IgG1, IgG2b, IgG2c, IgG3, IgG4, IgM, IgA1, IgA2, IgE, and/or IgD antibody isotypes. In another embodiment, the humoral response at least comprises the production of IgG1 antibody isotype.

Preferably, the dendritic cell is an animal dendritic cell or precursor of an animal dendritic cell. More preferably, the dendritic cell is a human dendritic cell. Even more preferably, the human dendritic cell is Necl-2+, HLA DR+ and/or BDCA-3+.

In yet another aspect, the present invention provides a method of treating and/or preventing a disease involving dendritic cells or precursors thereof, the method comprising administering to the subject a compound of the invention, and/or a composition of the invention.

Preferably, the method comprises administering a compound comprising the cytotoxic agent, drug and/or pharmacological agent.

In a further aspect, the present invention provides a method of treating and/or preventing a disease involving dendritic cells or precursors thereof, the method comprising administering to the subject an isolated polynucleotide and/or construct encoding said polynucleotide which, when present in a cell of the subject, modulates the level of activity of a polypeptide which comprises:

i) an amino acid sequence as provided in any one of SEQ ID NO's 48 to 80; and/or

ii) an amino acid sequence which is at least 50% identical to any one or more of SEQ ID NO's 48 to 80,

in the cell when compared to a cell that lacks said polynucleotide.

In an embodiment, the polynucleotide down-regulates the level of activity of the polypeptide in the cell. Examples of such polynucleotides include, but are not limited to, an antisense polynucleotide, a sense polynucleotide, a catalytic polynucleotide, a microRNA, and a double stranded RNA.

In an alternate embodiment, the polynucleotide up-regulates the level of activity of the polypeptide. For example, the polynucleotide encodes a polypeptide which comprises an amino acid sequence as provided in any one of SEQ ID NO's 48 to 80.

Examples of diseases involving dendritic cells or precursors thereof include, but are not limited to, cancer, an infection, an autoimmune disease or an allergy.

In an embodiment, the autoimmune disease is lupus erythematosus.

In another embodiment, the infection is a Plasmodium sp., such as Plasmodium falciparum or Plasmodium vivax, infection.

In another aspect, the present invention provides a method of modulating the uptake and/or clearance of cells with a disrupted cell membrane, cells infected with a pathogen, dying cells or dead cells, or a portion thereof, in a subject, the method comprising administering

i) a polypeptide comprising
a) an amino acid sequence as provided in any one of SEQ ID NO's 48 to 80;
b) an amino acid sequence which is at least 50% identical to any one or more of SEQ ID NO's 48 to 80; and/or
c) a biologically active fragment of a) or b),

ii) a compound of the invention, and/or

iii) a composition of the invention.

In another aspect, the present invention provides a method of modulating the antigen recognition, processing and/or presentation of material derived from cells with a disrupted cell membrane, cells infected with a pathogen, dying cells or dead cells, or a portion thereof, or surrounding cells, in a subject, the method comprising administering

i) a polypeptide comprising
a) an amino acid sequence as provided in any one of SEQ ID NO's 48 to 80;
b) an amino acid sequence which is at least 50% identical to any one or more of SEQ ID NO's 48 to 80; and/or
c) a biologically active fragment of a) or b),

ii) a compound of the invention, and/or

iii) a composition of the invention.

In a further aspect, the present invention provides a method of modulating an immune response to material derived from cells with a disrupted cell membrane, cells infected with a pathogen, dying cells or dead cells, or a portion thereof, or surrounding cells, in a subject, the method comprising administering a

i) a polypeptide comprising
a) an amino acid sequence as, provided in any one of SEQ ID NO's 48 to 80;
b) an amino acid sequence which is at least 50% identical to any one or more of SEQ ID NO's 48 to 80; and/or
c) a biologically active fragment of a) or b),

ii) a compound of the invention, and/or

iii) a composition of the invention.

In a further aspect, the present invention provides a method of modulating the uptake and/or clearance of cells with a disrupted cell membrane, cells infected with a pathogen, dying cells or dead cells, or a portion thereof, in a subject, the method comprising administering a compound which modulates the production of a polypeptide which comprises:

i) an amino acid sequence as provided in any one of SEQ ID NO's 48 to 80;

ii) an amino acid sequence which is at least 50% identical to any one or more of SEQ ID NO's 48 to 80; and/or

iii) a biologically active fragment of i) or ii).

In another aspect, the present invention provides a method of modulating the antigen recognition, processing and/or presentation of material derived from cells with a disrupted cell membrane, cells infected with a pathogen, dying cells or dead cells, or a portion thereof, or surrounding cells, in a subject, the method comprising administering a compound which modulates the production of a polypeptide which comprises:

i) an amino acid sequence as provided in any one of SEQ ID NO's 48 to 80;

ii) an amino acid sequence which is at least 50% identical to any one or more of SEQ ID NO's 48 to 80; and/or

iii) a biologically active fragment of i) or ii).

In a further aspect, the present invention provides a method of modulating an immune response to material derived from cells with a disrupted cell membrane, cells infected with a pathogen, dying cells or dead cells, or a portion thereof, or surrounding cells, in a subject, the method comprising administering a compound which modulates the production of a polypeptide which comprises:

i) an amino acid sequence as provided in any one of SEQ ID NO's 48 to 80;

ii) an amino acid sequence which is at least 50% identical to any one or more of SEQ ID NO's 48 to 80; and/or

iii) a biologically active fragment of i) or ii).

Preferably, the compound of the previous three aspects is a polynucleotide. Preferably, the polynucleotide is operably linked to a promoter capable of directing expression of the polynucleotide in a cell of an animal.

In an embodiment, the polynucleotide down-regulates mRNA levels from a gene encoding the polypeptide. Examples include, but are not limited to, an antisense polynucleotide, a sense polynucleotide, a catalytic polynucleotide, a microRNA, and a double stranded RNA.

In one embodiment, the polynucleotide is an antisense polynucleotide which hybridises under physiological conditions to a polynucleotide comprising any one or more of the sequence of nucleotides provided as SEQ ID NO's 81 to 113.

In another embodiment, the polynucleotide is a catalytic polynucleotide capable of cleaving a polynucleotide comprising any one or more of the sequence of nucleotides provided as SEQ ID NO's 81 to 113.

In a further embodiment, the polynucleotide is a double stranded RNA (dsRNA) molecule comprising an oligonucleotide which comprises at least 19 contiguous nucleotides of any one or more of the sequence of nucleotides provided as SEQ ID NO's 81 to 113, wherein the portion of the molecule that is double stranded is at least 19 basepairs in length and comprise's said oligonucleotide. In an embodiment, the polynucleotide is expressed from a single promoter, wherein the strands of the double stranded portion are linked by a single stranded portion.

In an alternate embodiment, the polynucleotide up-regulates mRNA levels from a gene encoding the polypeptide.

In one embodiment, the uptake and/or clearance of cells with a disrupted cell membrane, cells infected with a pathogen, dying cells or dead cells, or a portion thereof, is increased. In an alternate embodiment, the uptake and/or clearance of cells with a disrupted cell membrane, cells infected with a pathogen, dying cells or dead cells, or a portion thereof, is decreased.

In another embodiment, antigen recognition, processing and/or presentation of material derived from cells with a disrupted cell membrane, cells infected with a pathogen, dying cells, or dead cells, or a portion thereof, or surrounding cells, is increased. In an alternate embodiment, antigen recognition, processing and/or presentation of material derived from cells with a disrupted cell membrane, cells infected with a pathogen, dying cells, or dead cells, or a portion thereof, or surrounding cells, is decreased.

In another embodiment, the immune response to material derived from cells with a disrupted cell membrane, cells infected with a pathogen, dying cells or dead cells, or a portion thereof, or surrounding cells, is increased. In an alternate embodiment, the immune responses to material derived from cells with a disrupted cell membrane, cells infected with a pathogen, dying cells or dead cells, or a portion thereof, or surrounding cells, is decreased.

In a further preferred embodiment, the subject is suffering from a disease associated with cells with a disrupted cell membrane, cells infected with a pathogen, dying cells or dead cells, or a portion thereof. Examples of such diseases include, but are not limited to, graft versus host disease (GVHD), an autoimmune disease, an infection, a neurodegenerative disease, systemic inflammatory reaction syndrome (SIRS), cancer and injury.

Also provided is the use of a compound of the invention, and/or a composition of the invention for the manufacture of a medicament for modulating an immune response in a subject.

Furthermore, provided is the use of dendritic cells or precursors thereof exposed in vitro to a compound of the invention and/or a composition of the invention for the manufacture of a medicament for modulating an immune response to an antigen in a subject.

Also provided is the use of a compound of the invention and/or a composition of the invention for the manufacture of a medicament for treating and/or preventing a disease involving dendritic cells or precursors thereof in a subject.

In another aspect, provided is the use of

i) a polypeptide comprising
a) an amino acid sequence as provided in any one of SEQ ID NO's 48 to 80;
b) an amino acid sequence which is at least 50% identical to any one or more of SEQ ID NO's 48 to 80; and/or
c) a biologically active fragment of a) or b),

ii) a compound of the invention, and/or

iii) a composition of the invention, for the manufacture of a medicament for modulating the uptake and/or clearance of cells with a disrupted cell membrane, cells infected with a pathogen, dying cells or dead cells, or a portion thereof, in a subject.

In another aspect, provided is the use of

i) a polypeptide comprising
a) an amino acid sequence as provided in any one of SEQ ID NO's 48 to 80;
b) an amino acid sequence which is at least 50% identical to any one or more of SEQ ID NO's 48 to 80; and/or
c) a biologically active fragment of a) or b),

ii) a compound of the invention, and/or

iii) a composition of the invention,

for the manufacture of a medicament for modulating the antigen recognition, processing and/or presentation of material derived from cells with a disrupted cell membrane, cells infected with a pathogen, dying cells or dead cells, or a portion thereof, or surrounding cells, in a subject.

In another aspect, provided is the use of

i) a polypeptide comprising
a) an amino acid sequence as provided in any one of SEQ ID NO's 48 to 80;
b) an amino acid sequence which is at least 50% identical to any one or more of SEQ ID NO's 48 to 80; and/or
c) a biologically active fragment of a) or b),

ii) a compound of the invention, and/or

iii) a composition of the invention,

for the manufacture of a medicament for modulating an immune response to material derived from cells with a disrupted cell membrane, cells infected with a pathogen, dying cells or dead cells, or a portion thereof, or surrounding cells, in a subject.

In another aspect, provided is the use of a compound which modulates the production of a polypeptide which comprises:

i) an amino acid sequence as provided in any one of SEQ ID NO's 48 to 80;

ii) an amino acid sequence which is at least 50% identical to any one or more of SEQ ID NO's 48 to 80; and/or

iii) a biologically active fragment of i) or ii),

for the manufacture of a medicament for modulating the uptake and/or clearance of cells with a disrupted cell membrane, cells infected with a pathogen, dying cells or dead cells, or a portion thereof, in a subject.

In another aspect, provided is the use of a compound which modulates the production of a polypeptide which comprises:

i) an amino acid sequence as provided in any one of SEQ ID NO's 48 to 80;

ii) an amino acid sequence which is at least 50% identical to any one or more of SEQ ID NO's 48 to 80; and/or

iii) a biologically active fragment of i) or ii), for the manufacture of a medicament for modulating the antigen recognition, processing and/or presentation of material derived from cells with a disrupted cell membrane, cells infected with a pathogen, dying cells or dead cells, or a portion thereof, or surrounding cells, in a subject.

In another aspect, provided is the use of a compound which modulates the production of a polypeptide which comprises:

i) an amino acid sequence as provided in any one of SEQ ID NO's 48 to 80;

ii) an amino acid sequence which is at least 50% identical to any one or more of SEQ ID NO's 48 to 80; and/or

iii) a biologically active fragment of i) or ii),

for the manufacture of a medicament for modulating an immune response to material derived from cells with a disrupted cell membrane, cells infected with a pathogen, dying cells or dead cells, or a portion thereof, or surrounding cells, in a subject.

In a further aspect, the present invention provides a method of diagnosing, prognosing and/or monitoring the status of a disease associated with cells with a disrupted cell membrane, cells infected with a pathogen, dying cells or dead cells, the method comprising

i) contacting a cell with a compound that binds a polypeptide comprising
a) an amino acid sequence as provided in any one of SEQ ID NO's 48 to 80;
b) an amino acid sequence which is at least 50% identical to any one or more of SEQ ID NO's 48 to 80; and/or
c) a biologically active fragment of a) or b), and

ii) determining whether the polypeptide is present or absent,

wherein the presence of the polypeptide provides a diagnosis, prognosis and/or status of the disease.

In a preferred embodiment of the above aspect, the compound is not an antibody which binds Clec9A, Clec9A per se or a fragment of Clec9A which binds Clec9A such as a soluble fragment.

In an embodiment, the compound is an antibody or antigenic binding fragment thereof. Examples include, but are not limited to, a monoclonal antibody, humanized antibody, single chain antibody, diabody, triabody, or tetrabody.

In a preferred embodiment, the compound is detectably labelled.

In an embodiment, the method is performed in vivo on a subject. In an alternate embodiment, the method is performed in vitro on a sample obtained from a subject.

As noted above, examples of diseases associated with cells with a disrupted cell membrane, cells infected with a pathogen, dying cells or dead cells include, but are not limited to, graft versus host disease (GVHD), an autoimmune disease, an infection, a neurodegenerative disease, systemic inflammatory reaction syndrome (SIRS), cancer and injury.

In a further aspect, the present invention provides a method of monitoring the effectiveness of a therapy for killing a cell, the method comprising;

i) exposing a cell to the therapy, and

ii) detecting a cell with a disrupted cell membrane, dying cell or a dead cell, or a portion thereof, using a method of the invention,

wherein the presence of a cell with a disrupted cell membrane, a dying cell or a dead cell indicates that the therapy is effective,

In an embodiment, the cell in step i) is in vivo. In an alternate embodiment, the cell in step i) is in vitro.

Preferably, the therapy is administered to a subject. In an embodiment, the subject is suffering from a disease associated with cells with a disrupted cell membrane, cells infected with a pathogen, dying cells or dead cells. In a preferred embodiment, the subject has cancer or an infection.

In a further embodiment, step ii) is performed on a sample obtained from a subject.

The therapy can be any type of procedure. Examples include, but are not limited to, drug therapy or radiotherapy.

In a further aspect, the present invention provides a method of distinguishing between an early stage apoptotic cell and a late stage apoptotic cell, necrotic cell or dead cell, the method comprising

i) contacting a cell with a compound that binds
a) an amino acid sequence as provided in any one of SEQ ID NO's 48 to 80;
b) an amino acid sequence which is at least 50% identical to any one or more of SEQ ID NO's 48 to 80; and/or
c) a biologically active fragment of a) or b), and

ii) determining whether binding of the compound to the polypeptide is present or absent,

wherein the compound binding to the polypeptide indicates that the cell is a late stage apoptotic cell, necrotic cell or dead cell.

In a preferred embodiment of the above aspect, the compound is not an antibody which binds Clec9A, Clec9A per se or a fragment of Clec9A which binds Clec9A such as a soluble fragment.

In another aspect, the present invention provides a method of modulating an immune response to an antigen in a subject, the method comprising

i) obtaining a population of dendritic cells or precursors thereof,

ii) modulating the production and/or activity of a polypeptide which comprises:
a) an amino acid sequence as provided in any one of SEQ ID NO's 48 to 80;
b) an amino acid sequence which is at least 50% identical to any one or more of SEQ ID NO's 48 to 80; and/or
c) a biologically active fragment of a) or b),

iii) contacting the dendritic cells or precursors thereof with the antigen, and

iv) administering the dendritic cells or precursors thereof to the subject.

In a preferred embodiment of the above aspect, the activity of the polypeptide is not modulated by an antibody which binds Clec9A, Clec9A per se or a fragment of Clec9A which binds Clec9A such as a soluble fragment.

In a preferred embodiment, step iii) comprises contacting the dendritic cells or precursors thereof with a cell with a disrupted cell membrane, a cell infected with a pathogen, a dying cell, a dead cell, and/or a portion thereof, comprising said antigen.

In a further embodiment, step iii) comprises
a) obtaining a cell comprising the antigen,
b) disrupting the cell membrane of the cell, and
c) contacting the product of step b) with the dendritic cells or precursors thereof.

In another embodiment, the method further comprises, before step ii), enriching the population for cells expressing the polypeptide.

In an embodiment, steps ii) and iii) are conducted concurrently.

In an embodiment, the cell with a disrupted cell membrane, dying cell or dead cell is a cancer cell.

In another embodiment, the production and/or activity of the polypeptide is increased. In an alternate embodiment, the production and/or activity of the polypeptide is decreased.

In an embodiment, the precursor is a monocyte.

Preferably, an immune response to the antigen is increased.

In another aspect, the present invention provides a method of enriching dendritic cells, or a subset or precursors thereof, from a sample comprising;

i) contacting a sample comprising dendritic cells or precursors thereof with a compound of the invention, and

ii) isolating cells bound to the compound.

In a further aspect, the present invention provides a method of enriching dendritic cells, or a subset or precursors thereof, from a sample comprising;

i) contacting a sample comprising dendritic cells or precursors thereof with a detectably labelled first polynucleotide that hybridizes to a second polynucleotide encoding a polypeptide which comprises
a) an amino acid sequence as provided in any one of SEQ ID NO's 48 to 80; and/or
b) an amino acid sequence which is at least 50% identical to any one or more of SEQ ID NO's 48 to 80, and

ii) isolating the detectably labelled cells.

In a preferred embodiment, the cells obtained from step ii) of the two above methods' are administered to a subject. In an embodiment, the cells are administered to treat and/or prevent a disease selected from cancer, an infection, an autoimmune disease or an allergy.

The present invention also provides a method of detecting dendritic cells, or a subset or precursors thereof, in a sample comprising;

i) contacting a sample comprising dendritic cells or precursors thereof with a compound of the invention, and

ii) detecting cells bound to the compound.

In yet another aspect, the present invention provides a method of detecting dendritic cells, or a subset or precursor thereof, in a sample comprising;

i) contacting a sample comprising dendritic cells or precursors thereof with a detectably labelled first polynucleotide that hybridizes to a second polynucleotide encoding a polypeptide which comprises
a) an amino acid sequence as provided in any one of SEQ ID NO's 48 to 80; and/or
b) an amino acid sequence which is at least 50% identical to any one or more of SEQ ID NO's 48 to 80, and

ii) detecting the detectably labelled cells.

In another aspect, the present invention provides a method of detecting dendritic cells, or a subset or precursor thereof, in a subject comprising;

i) administering to the subject a compound of the invention,

ii) detecting cells bound to the compound.

In an embodiment, the compound is detectably labelled. However, as the skilled addressee will appreciate other procedures could be used, for example, using a detectably labelled secondary antibody that binds the compound.

In yet another aspect, the present invention provides a method of detecting dendritic cells, or a subset or precursor thereof, in a subject comprising;

i) administering to the subject a detectably labelled first polynucleotide that hybridizes to a second polynucleotide encoding a polypeptide which comprises
a) an amino acid sequence as provided in any one of SEQ ID NO's 48 to 80; and/or
b) an amino acid sequence which is at least 50% identical to any one or more of SEQ ID NO's 48 to 80, and,

ii) detecting the detectably labelled cells.

In a preferred embodiment, the dendritic cells express one or more of the following markers, CD8, CD24, Necl-2, CD11c, HLADR and BDCA3.

Preferably, the dendritic cells are human dendritic cells that express one or more of the following markers, Necl-2, HLADR and BDCA3.

In an alternative embodiment, the dendritic cells are murine dendritic cells that express one or more of the following markers, CD24, Necl-2, CD11c and CD8.

Preferably, the precursor dendritic cells are intermediate or late precursor dendritic cells which are capable of differentiating into dendritic cells in culture and/or on transfer into irradiated recipients.

In another aspect, the present invention provides a method of detecting a cell with a disrupted cell membrane, a cell infected with a pathogen, a dying cell or a dead cell, the method comprising

i) contacting a cell with a compound that binds a polypeptide comprising
a) an amino acid sequence as provided in any one of SEQ ID NO's 48 to 80;
b) an amino acid sequence which is at least 50% identical to any one or more of SEQ ID NO's 48 to 80; and/or
c) a biologically active fragment of a) or b), and

ii) determining whether binding of the compound to the polypeptide is present or absent,

wherein the compound binding to the polypeptide indicates that the cell has a disrupted cell membrane, is infected with a pathogen, is dying or is dead.

In a preferred embodiment of the above aspect, the compound is not an antibody which binds Clec9A, Clec9A per se or a fragment of Clec9A which binds Clec9A such as a soluble fragment.

Also provided is an isolated and/or exogenous polynucleotide encoding a compound of the invention, wherein the compound is a polypeptide.

In an embodiment, the polynucleotide comprises

i) a nucleotide sequence as provided in any one of SEQ ID NO's 81 to 113;

ii) a nucleotide sequence which is at least 50% identical to any one or more of SEQ ID NO's 81 to 113; and/or

iii) a nucleotide sequence which hybridizes to i) and/or ii), or a complement thereof.

In another aspect, the present invention provides an isolated polynucleotide which, when present in a cell of a subject, modulates the level of activity of a polypeptide in the cell when compared to a cell that lacks said polynucleotide, wherein the polypeptide comprises

i) an amino acid sequence as provided in any one of SEQ ID NO's 48 to 80;

ii) an amino acid sequence which is at least 50% identical to any one or more of SEQ ID NO's 48 to 80; and/or

iii) a biologically active fragment of i) or ii).

In an embodiment, the polynucleotide is operably linked to a promoter capable of directing expression of the polynucleotide in a cell of an animal.

In a preferred embodiment, the polynucleotide down-regulates mRNA levels from a gene encoding the polypeptide. Examples of such polynucleotides include, but are not limited to, an antisense polynucleotide, a sense polynucleotide, a catalytic polynucleotide, a microRNA and a double stranded RNA (dsRNA).

In an embodiment, the antisense polynucleotide hybridises under physiological conditions to a polynucleotide comprising any one or more of the sequence of nucleotides provided as SEQ ID NO's 81 to 113.

In another embodiment, the catalytic polynucleotide is capable of cleaving a polynucleotide comprising any one or more of the sequence of nucleotides provided as SEQ ID NO's 81 to 113.

In a further embodiment, the dsRNA molecule comprises an oligonucleotide which comprises at least 19 contiguous nucleotides of any one or more of the sequence of nucleotides provided as SEQ ID NO's 81 to 113 where T is replaced with a U, wherein the portion of the molecule that is double stranded is at least 19 basepairs in length and comprises said oligonucleotide.

In yet a further embodiment, the dsRNA molecule is expressed from a single promoter, wherein the strands of the double stranded portion are linked by a single stranded portion.

In an alternate embodiment, the polynucleotide up-regulates mRNA levels from a gene encoding the polypeptide. For example, the polynucleotide encodes the polypeptide.

Also provided is a vector comprising at least one polynucleotide of the invention. Preferably, the vector is an expression vector.

In a further aspect, the present invention provides a host cell comprising at least one polynucleotide of the invention, and/or at least one vector of the invention. The cell can be any cell type such as, but not limited to, a bacterial, yeast, animal, insect or plant cell.

Also provided are transgenic non-human organisms, such as transgenic plants, comprising at least one cell of the invention.

In a further aspect, the present invention provides an enriched population of dendritic cells and/or precursors thereof, obtained by a method of the invention.

In another aspect, the present invention provides an expanded dendritic cell population, and/or precursors thereof, obtained by culturing an enriched population of dendritic cells and/or precursors thereof of the invention.

In a further aspect, provided is a composition comprising a polynucleotide of the invention, a vector of the invention, a host cell of the invention, and/or a cell population of the invention, and a pharmaceutically acceptable carrier.

In a further aspect, provided is a kit comprising a compound of the invention, a polynucleotide of the invention, a vector of the invention, a host cell of the invention, a cell population of the invention and/or a composition of the invention.

As will be apparent, preferred features and characteristics of one aspect of the invention are applicable to many other aspects of the invention.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The invention is hereinafter described by way of the following non-limiting Examples and with reference to the accompanying figures.

### BRIEF DESCRIPTION OF THE ACCOMPANYING DRAWINGS

FIG. 1. The genomic structure and predicted protein structure encoded by the mouse (m) and human (h) 5B6 genes. The full-length cDNA encoding (A) mouse and (B) human 5B6. (C) Protein sequence alignment of the predicted protein sequence encoded by mouse and human 5B6. Sequence identity is highlighted in dark grey, similarity is shown in a light grey. Arrowheads denote exon boundaries. (D) Gene structures of mouse and human 5B6, determined by alignment of the cDNA to the genomic sequence databases of the C57BL/6J mouse (UCSC assembly February 2006) and human databases (UCSC assembly March 2006) respectively, are represented schematically. Exons encoding the coding region of 5B6 genes are denoted by black boxes and the size (bp) of the exons and introns are shown below. (E) A schematic representation of the mouse and human 5B6 proteins.

FIG. 2. Alignment of the CTLD of mouse and human 5B6 (Clec9A) to proteins that share sequence homology. Rat mannose binding protein A (MBP-A) is included for comparison as a classical C-type lectin domain that has functional carbohydrate recognition domains. Grey boxes indicate conserved residues, (+) indicates additional pair of cysteine residues involved in protein homodimeration, (*) marks the conserved cysteine residues that form disulfide bonds. The residues that ligate Ca²⁺ in the MBP-A are designated 1 and 2.

FIG. 3. Gene expression profiles of mouse 5B6. Real-time RT-PCR was used to study the expression profiles of the 5B6 gene relative to Gapdh in (A) lymphoid organ steady state DC including splenic cDC subsets; DN, CD4⁺ and CD8⁺, thymic cDC subsets; CD8⁻ and CD8⁺, LN cDC subsets; CD8⁻, CD8⁺, Dermal and Langerhans' cells (LC) and in thymic and splenic pDC. (B) Haemopoietic cells including thymocytes (thym), lymph node (LN) B and T cells, spleen (spl) B and T cells, NK cells, immature macrophages (im mac), mature macrophages (mat mac), splenic pDC and cDC. (C) Splenic cDC isolated from both steady state (resting) mice and after 3 hours in vivo activation with LPS and CpG.

FIG. 4. Surface expression of m5B6 (Clec9A) protein on DCs and other hemopoietic cells. (A) The DCs were purified and surface labeled by 4-color immunofluorescent staining. DCs were stained with mAb against CD11c (N418-PeCy7), CD45RA (14.8-APC), CD8 (53-6.7-APC-Cy7) and m5B6 (10B4-biotin). Splenic DCs were also stained with CD4 (GK1.5-FITC), thymic DCs with Sirpα (p84-FITC), and subcutaneous LN DCs with CD205 (NLDC-145-FITC). Splenic cDCs were divided into CD4⁺ cDCs (CD11^{hi}CD45RA⁻CD4⁺CD8⁻), DN cDCs (CD11^{hi}CD45RA⁻CD4⁻CD8⁻) and CD8⁺cDCs (CD11^{hi}CD45RA⁻CD8⁺CD4⁻); thymic cDCs were divided into CD8⁻cDCs (Sirpα^{hi}CD8^{lo}) and CD8⁺cDCs (Sirpα^{lo}CD8⁺); and LN cDCs into CD8⁻cDC (CD11c⁺CD205⁻CD8⁻), dermal cDCs (CD11c⁺CD205^{int}CD8⁻), Langerhans' cells (CD11c⁺CD205^{hi}CD8⁻) and CD8⁺cDCs (CD11c⁺CD205^{hi}CD8⁺), as described previously³¹. pDCs were identified as CD11c^{int}CD45RA⁺. Splenocytes were stained with mAb against CD3 (KT3-1.1-FITC), CD 19 (1D3-PeCy7), NK1.1 (PK136-PeCy7), CD49b (Hmα2-APC) and B cells (CD19⁺CD3⁻), T cells (CD19⁻ CD3⁺) and NK cells (NK1.1⁺CD49b⁺CD3⁻) were identified. Splenic macrophages were enriched as indicated in Materials and Methods and stained with CD11b (M1/70-Cy5) and F4/80-FITC and defined as CD11b^{hi}F4/80⁺. Bone marrow cells and splenocytes were stained with mAb against CD11b (M1/70-Cy5) and Ly6C (5075-3,6-FITC) and monocytes were defined as side-scatter^{lo}Ly6C^{hi}CD11b^{hi}. Bone marrow macrophages were Ly6C^{int}CD11b^{hi}. Cell populations were counterstained with SA-PE and analysed for m5B6 expression. The solid line represents m5B6 staining on gated cells, the dotted line represents staining of the gated cells with an isotype-matched control. (B) Enriched preparations of splenic DCs were stained with mAb against m5B6 (10B4-biotin), CD11c (N418-Quantum dot 655), CD8a (YTS-169-PercpCy5.5) and CD24 (M1/69-Alexa 633) and 120G8-FITC, then counterstained with SA-PE. pDCs (CD11c^{int}120G8⁺) and cDCs (CD11c^{hi}120G8⁻) were analysed for expression of m5B6. m5B6 expression correlated with CD8a and CD24 expression on cDCs. Most splenic pDCs expressed m5B6. (C) An enriched preparation of blood DCs was stained in parallel with the splenic DCs (B) using the same mAbs and analysed using identical gating strategies. Blood DCs do not express CD8α, but do express CD24. Similar to splenic DCs, blood DCs expressing CD24 also co-expressed 5B6 pDCs from the blood, like their splenic counterpart, expressed m5B6.

FIG. 5. Expression of 5B6 on human and macaque DC and hemopoietic cells. (A) Human and macaque peripheral blood mononuclear cells (PBMCs) were isolated and surface immunofluorescence labeled with mAb against HLADR, BDCA3, 5B6, and a PE-conjugated Lineage cocktail including CD3 (T cells), CD14 (monocytes), CD19 (B cells) and CD56 (Natural killer cells). Blood DC were gated as HLADR⁺ Lineage (PE)⁻ and further analysed for their expression of 5B6 (human and macaque) and BDCA3 (human). (B) Human PBMC were surface immunofluorescence labeled with mAb against the required surface markers and 5B6. Monocytes (CD14⁺), NK cells (NKp46⁺), T cells (CD3⁺), and B cells (CD19⁺) were gated and analysed for their expression of 5B6 (solid line). The dotted line represents staining of the gated cells with an isotype matched control.

FIG. 6. Binding of soluble 5B6 to membrane bound 5B6 on transiently transfected 293T cells. 293T cells were transiently transfected with expression constructs encoding full length untagged m5B6 (283T-m5B6), h5B6 (293T-h5B6) or no DNA (293T). Two days later, cells were harvested and surface immunofluorescence labeled using soluble FLAG-tagged m5B6, h5B6 and Cire, and binding detected using biotinylated anti-Flag mAb 9H10 and Streptavidin PE. Live cells were gated on forward scatter and propidium iodide exclusion and analysed for their surface binding of soluble 5B6 (solid line) relative to control staining with anti-Flag Ab and streptavidin-PE (dashed line).

FIG. 7. Generation of soluble recombinant ectodomains of Clec9A. (A) A schematic representation of the endogenous and recombinant soluble mClec9A proteins. The endogenous protein includes the Clec9A extracellular domains, the transmembrane (TM) and the cytoplasmic (cyto) domains. Two forms of recombinant soluble mClec9A protein were generated: mClec9A-ecto which consists of the full Clec9A ectodomain, a FLAG tag and a biotinylation consensus sequence (predicted mol wt ˜27 kDa); and mClec9A-CTLD which consists of the Clec9A-CTLD, FLAG tag and biotinylation consensus sequence (predicted mol wt ˜19.7 kDa). (B) Western blot analysis of endogenous mClec9A expression. DCs were produced from cultures of bone marrow with Flt3L (Naik et al., 2005) and DC lysates electrophoresed under non-reducing (N) and reducing (R) conditions. Blots were hybridised using anti-mClec9A Ab (24/04-10B4) and binding detected using HRP conjugated anti-rat Ig and Enhanced Chemiluminescence-Plus (Amersham). mClec9A was observed to migrate as a dimer under non-reducing conditions. (C) Western blot analysis of biotinylated recombinant soluble mClec9A protein. Biotinylated mClec9A-ecto and mClec9A-CTLD were electrophoresed under nonreducing (N) and reducing (R) conditions, and proteins detected using SA-HRP and Enhanced Chemiluminescence (Amersham). Recombinant mClec9A-ecto, like endogenous mClec9A, was observed to migrate as a dimer under nonreducing conditions and as a monomer under reducing conditions whereas mClec9A-CTLD migrated as a monomer under all conditions (B, C).

FIG. 8. Binding of Clec9A ectodomains to dead cells. (A) Thymocytes were γ-irradiated (5Gy) then cultured for 4 h or 16 h at 37° C. in RPMI-1640 containing 10% FCS, to follow the progress of apoptotic death. Samples were incubated with biotinylated mClec9A-ecto (solid line), or biotinylated Cire-ecto as a background control (dashed line) and binding detected using SA-PE. Thymocytes were stained with Annexin V-FITC, analysed by flow cytometry, and gated as Annexin V⁻ (viable) cells or Annexin V⁺(apoptotic) for analysis of Clec9A binding. At 4 h 41% of the Annexin V⁺ cells were PI⁺; at 16 h 90% of the Annexin V⁺ cells were PI⁺. The backgrounds observed using biotinylated Cire-ecto were similar to those observed with second stage reagents alone. (B) MEFs overexpressing Noxa to inactivate Mcl-1 (van Delft et al., 2006) were grown to approximately 80% confluence then induced to undergo apoptosis by treatment with 2.5 μM ABT-737 for 16 h. Control untreated MEFs (viable) and ABT-737 treated MEFs (late apoptotic) were harvested and incubated with mClec9A-ecto (solid line), hCLEC9A-ecto (solid line), or Cire-ecto (background control, dashed line). Binding was detected using biotinylated anti-FLAG mAb, SA-PE and flow cytometry. 90% of the untreated MEFs were viable based on normal forward scatter (FSC) and PI exclusion (PI⁻), whereas 95% of the ABT-737 treated MEFs were dead based on reduced FSC and positive PI staining. (C) Control untreated MEFs (viable) and ABT-737 treated MEFs (2.5 μM ABT-737 for 16 h; late apoptotic) were incubated in PBS alone or in the presence of DNaseI, RNaseA, protease K or trypsin. Cells were washed extensively to remove nucleases and proteases, then incubated with biotinylated mClec9A-ecto (solid line), or biotinylated Cire-ecto as a control (dashed line). Binding was detected using SA-PE and flow cytometry. 80% of the untreated MEFs were viable based on normal FSC and PI exclusion, whereas 97% of the ABT-737 treated MEFs were dead based on reduced FSC and positive PI staining.

FIG. 9. Clec9A binding is mediated via the CTLD and is directed against a protein expressed by diverse species. (A) Viable or freeze-thawed mouse fibroblasts (3T3 cell line) were incubated with biotinylated mClec9A or hCLEC9A ectodomains or CTLD (solid line), or with biotinylated control (Cire-ecto, dashed line). (B) Human 293T cells were freeze-thawed then incubated with biotinylated mClec9A-ecto, hCLEC9A-ecto (solid line) or Cire (dashed line) in the absence or presence of 5 mM EDTA (solid line). (C) Mouse 3T3 cells, insect SF21 cells, bacterial JM109 cells and yeast (Pichia pastoris) cells were freeze-thawed twice then incubated with biotinylated mClec9A-ecto (solid line), or biotinylated control (Cire-ecto, dashed line). Binding in (A)-(C) was detected using SA-PE and flow cytometry.

FIG. 10. Binding of Clec9A to a cytoskeletal component of red blood cell membranes. (A) Live RBC or RBC membranes (Saponin ghosts) were incubated with biotinylated mClec9A-ecto, mClec9A-CTLD (solid line) or with biotinylated control proteins mClec12A (solid line) or Cire (dashed line). (B) RBC membranes (Saponin ghosts) were treated in the absence (thick solid line) or presence of spectrin removal buffer (thin solid line) before incubating with biotinylated mClec9A-ecto, mClec9A-CTLD (solid line) or with the biotinylated control protein Cire (dashed line). Binding in (A)-(B) was detected using SA-PE and flow cytometry.

FIG. 11. Clec9A binds to purified spectrin. ELISA plates were coated with spectrin or actin protein (10 μg/ml), then probed with graded concentration of purified biotinylated Clec9A-ecto, Clec9A-CTLD or control protein Cire. Bound proteins were detected using SA-HRP and visualized using ABTS. The cumulative data of three experiments is presented. Clec9A-ecto bound spectrin more efficiently than Clec9A-CTLD. Neither Clec9A-ecto nor Clec9A-CTLD bound to actin. The control protein Cire did not bind spectrin or actin.

FIG. 12. Soluble mClec9A does not block uptake of dead cells by CD8⁺ DC. (A) Surface expression of Clec9A on splenic cDCs. DCs were blocked using rat Ig and anti FcR mAb (2.4G2) and surface labelled using mAb against CD11c (N418-PE-Cy7), CD45RA (14.8-APC), CD8 (53-6.7-APC-Cy7), CD172a (p84-FITC) and either Clec9A (24/04-10B4-biotin) or an isotype control-biotin (IgG2a-kappa; BD Pharmingen), then counterstained with SA-PE and analysed by flow cytometry. Splenic cDCs were gated as CD 11 C^{hi} CD45RA⁻ CD172a^{hi} CD8⁻ (CD8⁻) or as CD11c^{hi} CD45RA⁻ CD172a^{lo} CD8⁺ (CD8⁺) and analysed for their surface expression of Clec9A. The solid line represents mClec9A staining on gated cells, the dotted line represents staining of the gated cells using an isotype matched control. (B) Phagocytic uptake of dead cells by splenic cDCs. Splenocytes were freeze-thawed then labelled with PI, then incubated in the absence or presence of mClec9A-ecto. DCs were surface labelled with mAb against CD11c and CD8, then cocultured with the PI labelled splenocytes for 3 h at 4° C. or at 37° C. DCs were gated as CD8⁺ (CD11c^{hi} CD8⁺) or CD8⁻ (CD11c^{hi}CD8⁻) and analysed for the percentage of cells that were PI positive as a measure of dead cell uptake.

FIG. 13. A) mClec9A and hCLEC9A bind to RNF41. mClec9A-ecto, hClec9A-ecto and mClec12A-ecto were incubated with bead bound RNF41-fusion proteins. Bound proteins were eluted and detected using anti-Flag-HRP (lane 1:GST-RNF41_{FL}, lane 2: GST-RNF41₇₂₋₃₁₇, lane3: GST control). A sample of the purified Clec-ecto is shown in lane 4. B) Coomassie staining of GST-RNF41 fusion proteins eluted from glutathione beads.

### Key to the Sequence Listing

### SEQ ID NO:1—Human 5B6.

### SEQ ID NO:2—Murine 5B6.

### SEQ ID NO:3—Chimpanzee 5B6.

SEQ ID NO:4—Rhesus monkey 5B6.

### SEQ ID NO:5—Dog 5B6.

### SEQ ID NO:6—Cow 5B6.

### SEQ ID NO:7—Horse 5B6.

### SEQ ID NO:8—Rat 5B6.

SEQ ID NO:9—Open reading frame encoding human 5B6.
SEQ ID NO:10—Open reading frame encoding murine 5B6.
SEQ ID NO:11—Open reading frame encoding chimpanzee 5B6.
SEQ ID NO:12—Open reading frame encoding rhesus monkey 5B6.
SEQ ID NO:13—Open reading frame encoding dog 5B6.
SEQ ID NO:14—Open reading frame encoding cow 5B6.
SEQ ID NO:15—Open reading frame encoding horse 5B6.
SEQ ID NO:16—Open reading frame encoding rat 5B6.
SEQ ID NO's 17 to 28, 38 and 39—Oligonucleotide primers.
SEQ ID NO:29—Antigenic fragment of murine 5B6.
SEQ ID NO:30—Antigenic fragment of human 5B6.
SEQ ID NO:31—Biotinylation consensus sequence.
SEQ ID NO:32—Partial sequence of mouse Clec12a.
SEQ ID NO:33—Partial sequence of mouse Dectin-1.
SEQ ID NO:34—Partial sequence of mouse Clec8a.
SEQ ID NO:35—Partial sequence of mouse NKG2D.
SEQ ID NO:36—Partial sequence of human NKG2D.
SEQ ID NO:37—Partial sequence of rat MBP-A.
SEQ ID NO:40—Soluble mouse 5B6 including stalk.
SEQ ID NO:41—Soluble human 5B6 including stalk.
SEQ ID NO:42—Soluble mouse 5B6 without stalk.
SEQ ID NO:43—Soluble human 5B6 without stalk.
SEQ ID NO:44—Soluble flag tagged mouse 5B6 including stalk.
SEQ ID NO:45—Soluble flag tagged human 5B6 including stalk.
SEQ ID NO:46—Soluble flag tagged mouse 5B6 without stalk.
SEQ ID NO:47—Soluble flag tagged human 5B6 without stalk.
SEQ ID NO:48—Human erythrocytic spectrin, alpha 1 (elliptocytosis 2 or SPTA1).
SEQ ID NO:49—Human non-erythrocytic spectrin, alpha 1 (alpha-fodrin or SPTAN1) (isoform 1).
SEQ ID NO:50—Human non-erythrocytic spectrin, alpha 1 (alpha-fodrin or SPTAN1) (isoform 2).
SEQ ID NO:51—Human erythrocytic beta spectrin, or SPTB (isoform a).
SEQ ID NO:52—Human erythrocytic beta spectrin, or SPTB (isoform b).
SEQ ID NO:53—Human non-erythrocytic beta spectrin 1, or SPTBN1 (isoform 1).
SEQ ID NO:54—Human non-erythrocytic beta spectrin 1, or SPTBN1 (isoform 2).
SEQ ID NO:55—Human non-erythrocytic beta spectrin 2, or SPTBN2.
SEQ ID NO:56—Mouse spectrin alpha 1, or SPNA1.
SEQ ID NO:57—Mouse spectrin alpha 2, or SPNA2.
SEQ ID NO:58—Mouse spectrin beta 1, or SPNB1.
SEQ ID NO:59—Mouse spectrin beta 2, or SPNB2 (isoform 1).
SEQ ID NO:60—Mouse spectrin beta 2, or SPNB2 (isoform 2).
SEQ ID NO:61—Mouse spectrin beta 3, or SPNB3.
SEQ ID NO:62—Mouse spectrin beta 4, or SPNB4.
SEQ ID NO:63—Mouse spectrin beta 5, or SPNB5.
SEQ ID NO:64—Chimpanzee erythrocytic alpha 1 spectrin, (elliptocytosis 2 or SPTA1) (isoform 1).
SEQ ID NO:65—Chimpanzee erythrocytic alpha 1 spectrin, (elliptocytosis 2 or SPTA1) (isoform 2).
SEQ ID NO:66—Chimpanzee erythrocytic alpha 1 spectrin, (elliptocytosis 2 or SPTA1) (isoform 3).
SEQ ID NO:67—Chimpanzee erythrocytic beta spectrin, or SPTB (isoform 1).
SEQ ID NO:68—Chimpanzee erythrocytic beta spectrin, or SPTB (isoform 2).
SEQ ID NO:69—Chimpanzee erythrocytic beta spectrin, or SPTB (isoform 3).
SEQ ID NO:70—Chimpanzee erythrocytic beta spectrin, or SPTB (isoform 4).
SEQ ID NO:71—Chimpanzee non-erythrocytic beta spectrin 1, or SPTBN1 (isoform 1).
SEQ ID NO:72—Chimpanzee non-erythrocytic beta spectrin 2, or SPTBN2 (isoform 1).
SEQ ID NO:73—Horse erythrocytic alpha spectrin 1 (elliptocytosis 2 or SPTA1).
SEQ ID NO:74—Horse erythrocytic beta spectrin, or SPTB.
SEQ ID NO:75—Horse non-erythrocytic beta spectrin 1, or SPTBN1.
SEQ ID NO:76—Human RNF41 RING (Really Interesting New Gene) finger protein 41 (isoform 1).
SEQ ID NO:77—Human RNF41 RING (Really Interesting New Gene) finger protein 41 (isoform 2).
SEQ ID NO:78—Mouse RNF41 RING (Really Interesting New Gene) finger protein 41 (isoform 1).
SEQ ID NO:79—Chimpanzee RNF41 RING (Really Interesting New Gene) finger protein 41 (isoform 1).
SEQ ID NO:80—Horse RNF41 RING (Really Interesting New Gene) finger protein 41.
SEQ ID NO:81—Open reading frame encoding human erythrocytic spectrin, alpha 1 (ellipiocytosis 2 or SPTA1).
SEQ ID NO:82—Open reading frame encoding human non-erythrocytic spectrin, alpha 1 (alpha-fodrin or SPTAN1) (isoform 1).
SEQ ID NO:83—Open reading frame encoding human non-erythrocytic spectrin, alpha 1 (alpha-fodrin or SPTAN1) (isoform 2).
SEQ ID NO:84—Open reading frame encoding human erythrocytic beta spectrin, or SPTB (isoform a).
SEQ ID NO:85—Open reading frame encoding human erythrocytic beta spectrin, or SPTB (isoform b).
SEQ ID NO:86—Open reading frame encoding human non-erythrocytic beta spectrin 1, or SPTBN1 (isoform 1).
SEQ ID NO:87—Open reading frame encoding human non-erythrocytic beta spectrin 1, or SPTBN1 (isoform 2).
SEQ ID NO:88—Open reading frame encoding human non-erythrocytic beta spectrin 2, or SPTBN2.
SEQ ID NO:89—Open reading frame encoding mouse spectrin alpha 1, or SPNA1.
SEQ ID NO:90—Open reading frame encoding. mouse spectrin alpha 2, or SPNA2.
SEQ ID NO:91—Open reading frame encoding. mouse spectrin beta 1, or SPNB1.
SEQ ID NO:92—Open reading frame encoding mouse spectrin beta 2, or SPNB2 (isoform 1).
SEQ ID NO:93—Open reading frame encoding mouse spectrin beta 2, or SPNB2 (isoform 2).
SEQ ID NO:94—Open reading frame encoding mouse spectrin beta 3, or SPNB3.
SEQ ID NO:95—Open reading frame encoding mouse spectrin beta 4, or SPNB4.
SEQ ID NO:96—Open reading frame encoding mouse spectrin beta 5, or SPNB5.
SEQ ID NO:97—Open reading frame encoding chimpanzee erythrocytic alpha 1 spectrin, (elliptocytosis 2 or SPTA1) (isoform 1).
SEQ ID NO:98—Open reading frame encoding chimpanzee erythrocytic alpha 1 spectrin, (elliptocytosis 2 or SPTA1) (isoform 2).
SEQ ID NO:99—Open reading frame encoding chimpanzee erythrocytic alpha 1 spectrin, (elliptocytosis 2 or SPTA1) (isoform 3).
SEQ ID NO:100—Open reading frame encoding chimpanzee erythrocytic beta spectrin, or SPTB (isoform 1).
SEQ ID NO:101—Open reading frame encoding chimpanzee erythrocytic beta spectrin, or SPTB (isoform 2).
SEQ ID NO:102—Open reading frame encoding chimpanzee erythrocytic beta spectrin, or SPTB (isoform 3).
SEQ ID NO:103—Open reading frame encoding chimpanzee erythrocytic beta spectrin, or SPTB (isoform 4).
SEQ ID NO:104—Open reading frame encoding chimpanzee non-erythrocytic beta spectrin 1, or SPTBN1 (isoform 1).
SEQ ID NO:105—Open reading frame encoding chimpanzee non-erythrocytic beta spectrin 2, or SPTBN2 (isoform 1).
SEQ ID NO:106—Open reading frame encoding horse erythrocytic alpha spectrin 1 (elliptocytosis 2 or SPTA1).
SEQ ID NO:107—Open reading frame encoding horse erythrocytic beta spectrin, or SPTB.
SEQ ID NO:108—Open reading frame encoding horse non-erythrocytic beta spectrin 1, or SPTBN1.
SEQ ID NO:109—Open reading frame encoding human RNF41 RING (Really Interesting New Gene) finger protein 41 (isoform 1).
SEQ ID NO:110—Open reading frame encoding human RNF41 RING (Really Interesting New Gene) finger protein 41 (isoform 2).
SEQ ID NO:111—Open reading frame encoding mouse RNF41 RING (Really Interesting New Gene) finger protein 41 (isoform 1).
SEQ ID NO:112—Open reading frame encoding chimpanzee RNF41 RING (Really Interesting New Gene) finger protein 41 (isoform 1).
SEQ ID NO:113—Open reading frame encoding horse RNF41 RING (Really Interesting New Gene) finger protein 41.

### DETAILED DESCRIPTION OF THE INVENTION

### General Techniques and Definitions

Unless specifically defined otherwise, all technical and scientific terms used herein shall be taken to have the same meaning as commonly understood by one of ordinary skill in the art (e.g., in cell culture, molecular genetics, molecular biology, immunology, immunohistochemistry, protein chemistry, and biochemistry).

Unless otherwise indicated, the recombinant protein, cell culture, and immunological techniques utilized in the present invention are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

The phrase “cells with a disrupted cell membrane, cells infected with a pathogen, dying cells or dead cells”, and variations thereof, as used herein includes situations where the cell can have, where possible, one or more of these features. For example, the cell could have a disrupted membrane, be infected with a pathogen and be dying.

As used herein, the term “cells with a disrupted cell membrane” or “cell with a disrupted cell membrane” refers to cells where the integrity of the cell membrane has been compromised. This includes cells with pores, as well as damaged or ruptured cells.

As used herein, the term “dying cell” or “dying cells” refers to later stage apoptotic cells or necrotic cells. Preferably, the dying cell(s) are AnnexinV⁺ or they are propidium iodide (PI)⁺. For dying cells with a nuclei, it is preferred that they are AnnexinV⁺ and PI⁺. In a particularly preferred embodiment, the dying cells are at least AnnexinV⁺. In yet another embodiment, the necrotic cells are secondary necrotic cells.

As used herein, “early apoptotic cell” or “early apoptotic cells” includes cells that are AnnexinV⁺ and PI⁺.

As used herein, the term “dead cell” or “dead cells” refers to cell(s) that has passed a point of no return in the death process and which changes cannot be reversed. The cell(s) may have died through apoptosis or necrosis.

With regard to the phrase “cells infected with a pathogen”, the term pathogen includes any organism which can infect a cell. Examples include, but are not limited to, viruses, protozoa and bacteria.

As used herein, the term “or portion thereof” refers to any part of cells with a disrupted cell membrane, cells infected with a pathogen, dying cells or dead cells which comprise a ligand of a polypeptide defined herein. Examples include, but are not limited to, blebs and cell homogenates/lysates.

As used herein, the term “uptake and/or clearance” of cells with a disrupted cell membrane, cells infected with a pathogen, dying cells or dead cells, or a portion thereof, refers to the removal of cellular material, such a proteins or fragments thereof, of the cells. In an embodiment, dendritic cells are responsible, at least in part, for the uptake and/or clearance of the cells. Preferably, the dendritic cells are 5B6⁺ (also known as Clec9A⁺).

As used herein, the term “surrounding cells” refers to cells in close proximity to one or more of cells with a disrupted cell membrane, cells infected with a pathogen, dying cells or dead cells.

As used herein, the terms “treating”, “treat” or “treatment” include administering a therapeutically effective amount of a compound useful for the invention sufficient to reduce or eliminate at least one symptom of the specified condition.

As used herein, the terms “preventing”, “prevent” or “prevention” include administering a therapeutically effective amount of a compound useful for the invention sufficient to stop or hinder the development of at least one symptom of the specified condition.

As used herein, the term “diagnosing” or variations thereof refers to the detection of a disease.

As used herein, the term “prognosing” or variations thereof refers to an assessment of the future outcome of a disease.

As used herein, the term “monitoring the status” or variations thereof refers to determining the stage of a disease. The status can be determined before, during and/or after a subject has been administered with a treatment for the disease.

As used herein, the term “5B6” and “Clec9A” refers to a polypeptide which comprises;

i) an amino acid sequence as provided in any one of SEQ ID NO's 1 to 8;

ii) an amino acid sequence which is at least 50% identical to any one or more of SEQ ID NO's 1 to 8; and/or

iii) a biologically active, soluble and/or antigenic fragment of i) or ii). Preferably, the polypeptide is at least expressed on a subset of dendritic cells.

As used herein, in some embodiments the “sample” can be any biological material suspected of having cells with a disrupted cell membrane, cells infected with a pathogen, dying cells or dead cells, or a portion thereof. In other embodiments, the “sample” can be any biological material suspected of having 5B6+ dendritic cells Examples include, but are not limited to, blood, for example, whole peripheral blood, cord blood, fetus blood, bone marrow, plasma, serum, urine, cultured cells, saliva or urethral swab, lymphoid tissues, for example tonsils, peters patches, appendix, thymus, spleen and lymph nodes, and any biopsy samples taken for routine screening, diagnostic or surgical reason such as tumour biopsy or biopsy of inflamed organs/tissues. The sample may be tested directly or may require some form of treatment prior to testing. For example, a biopsy sample may require homogenization to produce a cell suspension prior to testing. Furthermore, to the extent that the biological sample is not in liquid form (for example, it may be a solid, semi-solid or a dehydrated liquid sample), it may require the addition of a reagent, such as a buffer, to mobilize the sample. The mobilizing reagent may be mixed with the sample prior to placing the sample in contact with a compound as defined herein.

As used herein, the terms “conjugate”, “conjugated” or variations thereof are used broadly to refer to any form to covalent or non-covalent association between a compound useful for the invention and a therapeutic agent or a detectable label, or to placing a compound useful for the invention and a therapeutic agent or detectable label in close proximity to each other such as in a liposome.

As used herein, the term “immune response” refers to an alteration in the reactivity of the immune system of a subject in response to an antigen and may involve antibody production, induction of cell-mediated immunity, complement activation and/or development of immunological tolerance.

As used herein, the phrase “disrupting the cell membrane of the cell” refers to any method that compromises the integrity of the cell membrane. Examples of such methods include, but are not limited to, irradiation, exposure to a detergent and freeze/thawing. In an embodiment, the cell is killed by the method.

As used herein, the term “subject” preferably relates to an animal. More preferably, the subject is a mammal such as a human, dog, cat, horse, cow, or sheep. Most preferably, the subject is a human.

### 5B6 (Clec9A) Ligands

As used herein, the term “5B6 ligand” or “Clec9A ligand” or variations thereof refers to a protein defined herein which binds 5B6 (Clec9A), namely spectrin, RNF41 as well as variants/mutants/fragments thereof.

### Spectrin

The term “spectrin” as used herein refers to membrane-associated cytoskeletal proteins involved in the crosslinking of filamentous actin which act as molecular scaffold proteins to link the actin cytoskeleton to the plasma membrane, and function in the determination of cell shape, arrangement of transmembrane proteins, and organization of organelles (Broderick and Winder, 2005).

The spectrins are traditionally divided into erythrocytic and non-erythrocytic forms, the former being exclusive to red blood cells and being responsible for the elasticity of RBCs. Spectrins are ubiquitous in cells and different isoforms may be expressed in different tissues in different organisms. Spectrins are highly modular proteins, containing many repeating alpha-helical 106-amino acid units (or ‘spectrin repeats’).

Alpha forms generally contain 20 spectrin repeats and, in contrast to the beta forms, generally lack an actin-binding domain (ABD). Most alpha forms contain and SH3 (Src homology 3 domains) for binding polyproline-containing proteins. Non-erythrocytic alpha isoforms generally contain an EF-hand motif for binding calcium. Examples of erythrocytic alpha forms of spectrin are given as SEQ ID Nos: 48, 64-66 and 73. Examples of non-erythrocytic alpha forms of spectrin are given as SEQ ID Nos: 49-50. Mutations in the human SPTA1 gene (encoding erythrocytic spectrin alpha 1) are the cause of elliptocytosis type 2 (EL2), an autosomal dominant hematological disorder characterised by hemolytic anemia and elliptical or oval RBC shape. SPTA1 mutations also cause hereditary pyropoikilocytosis (HPP) and spherocytosis type III (SPH3), both being hemolytic disorders. Mutations in the non-erythrocytic alpha 1 gene (SPTAN1) cause Sjogrens syndrome, autoimmune diseases, rheumatoid arthritis, multiple sclerosis, neurodegenerative diseases and xerostomia. Non-erythrocytic forms of alpha 1 spectrin (encoded by the SPTAN1 gene) are also known as alpha-fodrin.

Beta forms generally contain 17 spectrin repeats and an actin-binding domain (ABD). ABDs generally contain two CH (calponin homology) domains, which enable beta forms of spectrin to interact with F-actin. Non-erythrocytic forms of beta spectrin contain a PH (pleckstrin homology) domain for interaction with membrane phospholipids. Beta forms of spectrin generally lack EF-hand motifs. Examples of erythrocytic beta forms of spectrin are given as SEQ ID Nos:51-52, 67-70 and 74. Examples of non-erythrocytic beta forms of spectrin are given as SEQ ID Nos: 53-55, 71-72 and 75. Mutations in the human SPTB gene (encoding erythrocytic spectrin beta) are the cause of RBC disorders including elliptocytosis type 3 (EL3), spherocytosis type I (SPH1), muscular dystrophy, various anemic disease and pyropoikilocytosis. Mutations in the non-erythrocytic beta 1 gene (SPTBN1) cause neurofibromatosis type 2 and leukemia. Non-erythrocytic forms of beta 1 spectrin (encoded by the SPTBN1 gene) are also known as beta-fodrin.

Spectrin functions as a tetramer of alpha and beta dimers linked in a head-to-head arrangement. Alpha and beta spectrin interact to form a dimer and two heterodimers form the functional tetramer. Tetramers bind via their tail ends to a junctional complex consisting of filamentous actin and band 4.1 protein. Spectrin also binds to integral membrane proteins via ankyrin and band 3 protein (especially in RBCS) and also via protein 4.1 and glycophorin C. Interactions also occur with phospholipids via the PH domains of beta spectrin.

### RNF-41 Protein

RNF-41 protein is also known as RING (Really Interesting New Gene) finger protein, neuregulin receptor degradation protein-1 (NRDP1), or fetal liver RING protein (FLRF), refers to a protein which acts as an E3-ubiquitin ligase and regulates the degradation of target proteins. Target proteins for RNF-41 include members of the EGF (epidermal growth factor) receptor family, for example ErbB3 (or Her3). Other targets of RNF_{—}41 include ErbB4, ubiquitin-specific protease 8 (Usp8), Birch and reticulon 4 (Rtn4, also known as NogoA). Mutations in RNF-41 have been linked to tumour diseases. Overexpression of RNF-41 has been shown to decrease ErbB3 and inhibition of breast cancer growth. Decreased levels of RNF-41 are inversely correlated with ErbB3 levels in primary human breast cancer tissue.

In humans, three transcript variants encode 2 isoforms of RNF-41, namely isoform 1 and 2, given in SEQ ID Nos:76 and 77, respectively. Examples of RNF-41 proteins from other organisms are given in SEQ ID Nos: 78-80.

### Compounds

The present inventors have previously shown that 5B6 (also referred to in the art as CLEC9A and HEEE9341) is expressed in a subset of dendritic cells, can be targeted to modulate an immune response, and binds a ligand on cells with a disrupted cell membrane, cells infected with a pathogen, dying cells and dead cells (Caminschi et al., 2008; WO 09/026,660; U.S. 61/052,983; WO 09/137,871; U.S. 61/120,801). This enabled compounds which modulate the binding of 5B6 to the ligand, and/or compounds which modulate the production the ligand to be used in a wide variety of diagnostic, prognostic and therapeutic procedures. The present inventors have now identified further molecules which bind 5B6 which can be used to, inter alia, target therapeutic molecules to dendritic cells.

Compounds useful for the invention include the ligands such as spectrin or RNF41 modified to deliver a therapeutic agent, as well as those which bind, preferably which specifically bind, these ligands. The binding may be mediated by covalent or non-covalent interactions or a combination of covalent and non-covalent interactions. When the interaction produces a non-covalently bound complex, the binding which occurs is typically electrostatic, hydrogen-bonding, or the result of hydrophilic/lipophilic interactions. In a preferred embodiment, the compound is a purified and/or recombinant polypeptide.

Although not essential, the compound may bind specifically to 5B6 or the ligand. The phrase “specifically binds”, means that under particular conditions, the compound binds 5B6 or the ligand and does not bind to a significant amount to other, for example, proteins or carbohydrates. In one embodiment, the compound specifically binds 5B6 and not other molecules in a sample obtained from a subject comprising dendritic cells. In another embodiment, the compound specifically binds the ligand and not other molecules in a sample obtained from a subject comprising cells with a disrupted cell membrane, cells infected with a pathogen, dying cells or dead cells, or a portion thereof. Specific binding under such conditions may require an antibody that is selected for its specificity. In another embodiment, a compound is considered to “specifically binds” if there is a greater than 5-fold difference, and preferably a 25, 50 or 100 fold greater difference between the binding of the compound when compared to another protein.

### Antibodies

In one embodiment, the compound that binds the ligand, such as spectrin or RNF41, comprises an antibody or antigen binding fragment thereof. The terms “antibodies” and “immunoglobulin” refers to a class of structurally related glycoproteins consisting of two pairs of polypeptide chains, one pair of light (L) low molecular weight chains and one pair of heavy (H) chains, all four inter-connected by disulfide bonds. The structure of immunoglobulins has been well characterized, see for instance Fundamental Immunology Ch. 7 (Paul, W., ed., 2nd ed. Raven Press, N.Y. (1989)). Briefly, each heavy chain typically is comprised of a heavy chain variable region (abbreviated herein as V_{H}) and a heavy chain constant region (abbreviated herein as C_{H}). The heavy chain constant region typically is comprised of three domains, C_{H}1, C_{H}2, and C_{H}3. Each light chain typically is comprised of a light chain variable region (abbreviated herein as V_{L}) and a light chain constant region (abbreviated herein as C_{L}). The light chain constant region typically is comprised of one domain, C_{L}. The V_{H} and V_{L} regions may be further subdivided into regions of hypervariability (or hypervariable regions which may be hypervariable in sequence and/or form of structurally defined loops), also termed complementarity determining regions (CDRs), interspersed with regions that are more conserved, termed framework regions (FRs).

Each V_{H} and V_{L} is typically composed of three CDRs and four FRs, arranged from amino-terminus to carboxy-terminus in the following order: FR1, CDR1, FR2, CDR2, FR3, CDR3, FR4 (see also Chothia and Lesk, 1987). Typically, the numbering of amino acid residues in this region is performed by the method described in Kabat et al., Sequences of Proteins of Immunological Interest, 5th Ed. Public Health Service, National Institutes of Health, Bethesda, Md. (1991) (phrases such as variable domain residue numbering as in Kabat or according to Kabat herein refer to this numbering system for heavy chain variable domains or light chain variable domains). Using this numbering system, the actual linear amino acid sequence of a peptide may contain fewer or additional amino acids corresponding to a shortening of, or insertion into, a FR or CDR of the variable domain.

The term “humanized antibody”, as used herein, refers to herein an antibody derived from a non-human antibody, typically murine, that retains or substantially retains the antigen-binding properties of the parent antibody but which is less immunogenic in humans.

The term complementarity determining region (CDR), as used herein, refers to amino acid sequences which together define the binding affinity and specificity of a variable fragment (Fv) region of a immunoglobulin binding site.

The term framework region (FR), as used herein, refers to amino acid sequences interposed between CDRs. These portions of the antibody serve to hold the CDRs in appropriate orientation (allows for CDRs to bind antigen). A variable region, either light or heavy, comprises a framework and typically three CDRs.

The term constant region (CR) as used herein, refers to the portion of the antibody molecule which confers effector functions. The constant regions of the subject humanized antibodies are derived from human immunoglobulins. The heavy chain constant region can be selected from any of the five isotypes: alpha, delta, epsilon, gamma or mu. Further, heavy chains of various subclasses (such as the IgG subclasses of heavy chains) are responsible for different effector functions and thus, by choosing the desired heavy chain constant region, antibodies with desired effector function can be produced. Preferred heavy chain constant regions are gamma 1 (IgG1), gamma 2 (IgG2), gamma 3 (IgG3) and gamma 4 (IgG4), more preferably gamma 4 (IgG4). The light chain constant region can be of the kappa or lambda type, preferably of the kappa type.

Antibodies may exist as intact immunoglobulins, or as modifications in a variety of forms including, for example, but not limited to, domain antibodies including either the VH or VL domain, a dimer of the heavy chain variable region (VHH, as described for a camelid), a dimer of the light chain variable region (VLL), Fv fragments containing only the light and heavy chain variable regions, or Fd fragments containing the heavy chain variable region and the CH1 domain. A scFv consisting of the variable regions of the heavy and light chains linked together to form a single-chain antibody (Bird et al., 1988; Huston et al., 1988) and oligomers of scFvs such as diabodies and triabodies are also encompassed by the term “antibody”. Also encompassed are fragments of antibodies such as Fab, (Fab′)₂ and FabFc₂ fragments which contain the variable regions and parts of the constant regions. CDR-grafted antibody fragments and oligomers of antibody fragments are also encompassed. The heavy and light chain components of an Fv may be derived from the same antibody or different antibodies thereby producing a chimeric Fv region. The antibody may be of animal (for example mouse, rabbit or rat) or human origin or may be chimeric (Morrison et al., 1984) or humanized (Jones et al., 1986), and UK 8707252). As used herein the term “antibody” includes these various forms. Using the guidelines provided herein and those methods well known to those skilled in the art which are described in the references cited above and in such publications as Harlow & Lane (supra) the antibodies for use in the methods of the present invention can be readily made.

As used herein, an “antigenic binding fragment” refers to a portion of an antibody as defined herein that is capable of binding the same antigen as the full length molecule.

Antibodies or antigen binding fragments of the invention which are not from a natural source, such as a humanized antibody, preferably retain a significant proportion of the binding properties of the parent antibody. In particular, such antibodies or fragments of the invention retain the ability to specifically bind the antigen recognized by the parent antibody used to produce the antibody or fragment such as a humanized antibody. Preferably, the antibody or fragment exhibits the same or substantially the same antigen-binding affinity and avidity as the parent antibody. Ideally, the affinity of the antibody or fragment will not be less than 10% of the parent antibody affinity, more preferably not less than about 30%, and most preferably the affinity will not be less than 50% of the parent antibody. Methods for assaying antigen-binding affinity are well known in the art and include half-maximal binding assays, competition assays, and Scatchard analysis.

A variety of immunoassay formats may be used to select antibodies or fragments that are specifically immunoreactive with the ligand. For example, surface labelling and flow cytometric analysis or solid-phase ELISA immunoassays are routinely used to select antibodies specifically immunoreactive with a protein or carbohydrate. See Harlow & Lane (supra) for a description of immunoassay formats and conditions that can be used to determine specific immunoreactivity.

The antibodies may be Fv regions comprising a variable light (V_{L}) and a variable heavy (V_{H}) chain. The light and heavy chains may be joined directly or through a linker. As used herein a linker refers to a molecule that is covalently linked to the light and heavy chain and provides enough spacing and flexibility between the two chains such that they are able to achieve a conformation in which they are capable of specifically binding the epitope to which they are directed. Protein linkers are particularly preferred as they may be expressed as an intrinsic component of the Ig portion of the fusion polypeptide.

In another embodiment, recombinantly produced single chain scFv antibody, preferably a humanized scFv, is used in the methods of the invention.

### Monoclonal Antibodies

Monoclonal antibodies directed against the 5B6 ligands described herein can be readily produced by one skilled in the art.

The general methodology for making monoclonal antibodies by hybridomas is well known. Immortal antibody-producing cell lines can be created by cell fusion, and also by other techniques such as direct transformation of B lymphocytes with oncogenic DNA, or transfection with Epstein-Barr virus. Panels of monoclonal antibodies produced against 5B6 ligand epitopes can be screened for various properties; i.e. for isotype and epitope affinity.

Animal-derived monoclonal antibodies can be used for both direct in vivo and extracorporeal immunotherapy. However, it has been observed that when, for example, mouse-derived monoclonal antibodies are used in humans as therapeutic agents, the patient produces human anti-mouse antibodies. Thus, animal-derived monoclonal antibodies are not preferred for therapy, especially for long term use. With established genetic engineering techniques it is possible, however, to create chimeric or humanized antibodies that have animal-derived and human-derived portions. The animal can be, for example, a mouse or other rodent such as a rat.

If the variable region of the chimeric antibody is, for example, mouse-derived while the constant region is human-derived, the chimeric antibody will generally be less immunogenic than a “pure” mouse-derived monoclonal antibody. These chimeric antibodies would likely be more suited for therapeutic use, should it turn out that “pure” mouse-derived antibodies are unsuitable.

Methodologies for generating chimeric antibodies are available to those in the art. For example, the light and heavy chains can be expressed separately, using, for example, immunoglobulin light chain and immunoglobulin heavy chains in separate plasmids. These can then be purified and assembled in vitro into complete antibodies; methodologies for accomplishing such assembly have been described (see, for example, Sun et al., 1986). Such a DNA, construct may comprise DNA encoding functionally rearranged genes for the variable region of a light or heavy chain of an antibody linked to DNA encoding a human constant region. Lymphoid cells such as myelomas or hybridomas transfected with the DNA constructs for light and heavy chain can express and assemble the antibody chains.

In vitro reaction parameters for the formation of IgG antibodies from reduced isolated light and heavy chains have also been described. Co-expression of light and heavy chains in the same cells to achieve intracellular association and linkage of heavy and light chains into complete H2L2 IgG antibodies is also possible. Such co-expression can be accomplished using either the same or different plasmids in the same host cell.

In another preferred embodiment of the present invention the antibody is humanized, that is, an antibody produced by molecular modeling techniques wherein the human content of the antibody is maximised while causing little or no loss of binding affinity attributable to the variable region of, for example, a parental rat, rabbit or murine antibody.

There are several factors to consider in deciding which human antibody sequence to use during the humanisation. The humanisation of light and heavy chains are considered independently of one another, but the reasoning is basically similar for each.

This selection process is based on the following rationale: A given antibody's antigen specificity and affinity is primarily determined by the amino acid sequence of the variable region CDRs. Variable domain framework residues have little or no direct contribution. The primary function of the framework regions is to hold the CDRs in their proper spatial orientation to recognize antigen. Thus the substitution of animal, for example, rodent CDRs into a human variable domain framework is most likely to result in retention of their correct spatial orientation if the human variable domain framework is highly homologous to the animal variable domain from which they originated. A human variable domain should preferably be chosen therefore that is highly homologous to the animal variable domain(s). A suitable human antibody variable domain sequence can be selected as follow.

Step 1. Using a computer program, search all available protein (and DNA) databases for those human antibody variable domain sequences that are most homologous to the animal-derived antibody variable domains. The output of a suitable program is a list of sequences most homologous to the animal-derived antibody, the percent homology to each sequence, and an alignment of each sequence to the animal-derived sequence. This is done independently for both the heavy and light chain variable domain sequences. The above analyses are more easily accomplished if only human immunoglobulin sequences are included.

Step 2. List the human antibody variable domain sequences and compare for homology. Primarily the comparison is performed on length of CDRs, except CDR3 of the heavy chain which is quite variable. Human heavy chains and Kappa and Lambda light chains are divided into subgroups; Heavy chain 3 subgroups, Kappa chain 4 subgroups, Lambda chain 6 subgroups. The CDR sizes within each subgroup are similar but vary between subgroups. It is usually possible to match an animal-derived antibody CDR to one of the human subgroups as a first approximation of homology. Antibodies bearing CDRs of similar length are then compared for amino acid sequence homology, especially within the CDRs, but also in the surrounding framework regions. The human variable domain which is most homologous is chosen as the framework for humanisation.

### The Actual Humanising Methodologies/Techniques

An antibody may be humanized by grafting the desired CDRs onto a human framework according to EP 0239400. A DNA sequence encoding the desired reshaped antibody can therefore be made beginning with the human DNA whose CDRs it is wished to reshape. The animal-derived variable domain amino acid sequence containing the desired CDRs is compared to that of the chosen human antibody variable domain sequence. The residues in the human variable domain are marked that need to be changed to the corresponding residue in the animal to make the human variable region incorporate the animal-derived CDRs. There may also be residues that need substituting in, adding to or deleting from the human sequence.

Oligonucleotides are synthesized that can be used to mutagenize the human variable domain framework to contain the desired residues. Those oligonucleotides can be of any convenient size. One is normally only limited in length by the capabilities of the particular synthesizer one has available. The method of oligonucleotide-directed in vitro mutagenesis is well known.

Synthetic gene sequences, such as those encoding humanized antibodies or fragments thereof, can be commercially ordered through any of a number of service companies, including DNA 2.0 (Menlo Park, Calif.), Geneart (Regensburg, Germany), CODA Genomics (Irvine, Calif.), and GenScript, Corporation (Piscataway, N.J.).

Alternatively, humanisation may be achieved using the recombinant polymerase chain reaction (PCR) methodology of WO 92/07075. Using this methodology, a CDR may be spliced between the framework regions of a human antibody. In general, the technique of WO 92/07075 can be performed using a template comprising two human framework regions, AB and CD, and between them, the CDR which is to be replaced by a donor CDR. Primers A and B are used to amplify the framework region AB, and primers C and D used to amplify the framework region CD. However, the primers B and C each also contain, at their 5′ ends, an additional sequence corresponding to all or at least part of the donor CDR sequence. Primers B and C overlap by a length sufficient to permit annealing of their 5′ ends to each other under conditions which allow a PCR to be performed. Thus, the amplified regions AB and CD may undergo gene splicing by overlap extension to produce the humanized product in a single reaction.

Following the mutagenesis reactions to reshape the antibody, the mutagenised DNAs can be linked to an appropriate DNA encoding a light or heavy chain constant region, cloned into an expression vector, and transfected into host cells, preferably mammalian cells. These steps can be carried out in routine fashion. A reshaped antibody may therefore be prepared by a process comprising:

(a) preparing a first replicable expression vector including a suitable promoter operably linked to a DNA sequence which encodes at least a variable domain of an Ig heavy or light chain, the variable domain comprising framework regions from a human antibody and the CDRs required for the humanized-antibody of the invention;

(b) preparing a second replicable expression vector including a suitable promoter operably linked to a DNA sequence which encodes at least the variable domain of a complementary Ig light or'heavy chain respectively;

(c) transforming a cell line with the first or both prepared vectors; and

(d) culturing said transformed cell line to produce said altered antibody.

Preferably the DNA sequence in step (a) encodes both the variable domain and each constant domain of the human antibody chain. The humanized antibody can be prepared using any suitable recombinant expression system. The cell line which is transformed to produce the altered antibody may be a Chinese Hamster Ovary (CHO) cell line or an immortalised mammalian cell line, which is advantageously of lymphoid origin, such as a myeloma, hybridoma, trioma or quadroma cell line. The cell line may also comprise a normal lymphoid cell, such as a B-cell, which has been immortalised by transformation with a virus, such as the Epstein-Barr virus. Most preferably, the immortalised cell line is a myeloma cell line or a derivative thereof.

The CHO cells used for expression of the antibodies may be dihydrofolate reductase (dhfr) deficient and so dependent on thymidine and hypoxanthine for growth. The parental dhfr⁻ CHO cell line is transfected with the DNA encoding the antibody and dhfr gene which enables selection of CHO cell transformants of dhfr positive phenotype. Selection is carried out by culturing the colonies on media devoid of thymidine and hypoxanthine, the absence of which prevents untransformed cells from growing and transformed cells from resalvaging the folate pathway and thus bypassing the selection system. These transformants usually express low levels of the DNA of interest by virtue of co-integration of transfected DNA of interest and DNA encoding dhfr. The expression levels of the DNA encoding the antibody may be increased by amplification using methotrexate (MTX). This drug is a direct inhibitor of the enzyme dhfr and allows isolation of resistant colonies which amplify their dhfr gene copy number sufficiently to survive under these conditions. Since the DNA sequences encoding dhfr and the antibody are closely linked in the original transformants, there is usually concomitant amplification, and therefore increased expression of the desired antibody.

Another preferred expression system for use with CHO or myeloma cells is the glutamine synthetase (GS) amplification system described in WO 87/04462. This system involves the transfection of a cell with DNA encoding the enzyme GS and with DNA encoding the desired antibody. Cells are then selected which grow in glutamine free medium and can thus be assumed to have integrated the DNA encoding GS. These selected clones are then subjected to inhibition of the enzyme GS using methionine sulphoximine (Msx). The cells, in order to survive, will amplify the DNA encoding GS with concomitant amplification of the DNA encoding the antibody.

Although the cell line used to produce the humanized antibody is preferably a mammalian cell line, any other suitable cell line, such as a bacterial cell line or a yeast cell line, may alternatively be used. In particular, it is envisaged that E. coli-derived bacterial strains could be used. The antibody obtained is checked for functionality. If functionality is lost, it is necessary to return to step (2) and alter the framework of the antibody.

Once expressed, the whole antibodies, their dimers, individual light and heavy chains, or other immunoglobulin forms can be recovered and purified according to standard procedures of the art, including ammonium sulfate precipitation, affinity columns, column chromatography, gel electrophoresis and the like (See, generally, Scopes, R., Protein Purification, Springer-Verlag, N.Y. (1982)). Substantially pure immunoglobulins of at least about 90 to 95% homogeneity are preferred, and 98 to 99% or more homogeneity most preferred, for pharmaceutical uses. Once purified, partially or to homogeneity as desired, a humanized antibody may then be used therapeutically or in developing and performing assay procedures, immunofluorescent stainings, and the like (See, generally, Lefkovits and Pernis (editors), Immunological Methods, Vols. I and II, Academic Press, (1979 and 1981)).

Studies carried out by Greenwood et al. (1993) have demonstrated that recognition of the Fc region of an antibody by human effector cells can be optimised by engineering the constant region of the immunoglobulin molecule. This could be achieved by fusing the variable region genes of the antibody, with the desired specificity, to human constant region genes encoding immunoglobulin isotypes that have demonstrated effective antigen dependent cellular cytotoxicity (ADCC) in human subjects, for example the IgG1 and IgG3 isotypes (Greenwood and Clark, Protein Engineering of Antibody Molecules for Prophylactic and Therapeutic Applications in Man. Mike Clark (editor), Academic Titles, Section II, p. 85-113, (1993)). The resulting chimeric or humanized antibodies should be particularly effective in modulating humoral immunity and/or T-cell mediated immunity.

Antibodies with fully human variable regions can also be prepared by administering the antigen to a transgenic animal which has been modified to produce such antibodies in response to antigenic challenge, but whose endogenous loci have been disabled. Various subsequent manipulations can be performed to obtain either antibodies per se or analogs thereof (see, for example, U.S. Pat. No. 6,075,181).

### Preparation of Genes Encoding Antibodies or Fragments Thereof

Genes encoding antibodies, both light and heavy chain genes or portions thereof, e.g., single chain Fv regions, may be cloned from a hybridoma cell line. They may all be cloned using the same general strategy such as RACE using a commercially available kit, for example as produced by Clontech. Typically, for example, poly(A)⁺mRNA extracted from the hybridoma cells is reverse transcribed using random hexamers as primers. For Fv regions, the V_{H} and V_{L} domains are amplified separately by two polymerase chain reactions (PCR). Heavy chain sequences may be amplified using 5′ end primers which are designed according to the amino-terminal protein sequences of the anti-5B6 ligand heavy chains respectively and 3′ end primers according to consensus immunoglobulin constant region sequences (Kabat et al., Sequences of Proteins of Immunological Interest. 5th edition. U.S. Department of Health and Human Services, Public Health Service, National Institutes of Health, Bethesda, Md. (1991)). Light chain Fv regions are amplified using 5′ end primers designed according to the amino-terminal protein sequences of anti-5B6 ligand light chains and in combination with the primer C-kappa. One of skill in the art would recognize that many suitable primers may be employed to obtain Fv regions.

The PCR products are subcloned into a suitable cloning vector. Clones containing the correct size insert by DNA restriction are identified. The nucleotide sequence of the heavy or light chain coding regions may then be determined from double stranded plasmid DNA using sequencing primers adjacent to the cloning site. Commercially available kits (e.g., the Sequenase™ kit, United States Biochemical Corp., Cleveland, Ohio, USA) may be used to facilitate sequencing the DNA. DNA encoding the Fv regions may be prepared by any suitable method, including, for example, amplification techniques such as PCR and LCR.

Chemical synthesis produces a single stranded oligonucleotide. This may be converted into double stranded DNA by hybridization with a complementary sequence, or by polymerization with a DNA polymerase using the single strand as a template. While it is possible to chemically synthesize an entire single chain Fv region, it is preferable to synthesize a number of shorter sequences (about 100 to 150 bases) that are later ligated together.

Alternatively, sub-sequences may be cloned and the appropriate subsequences cleaved using appropriate restriction enzymes. The fragments may then be ligated to produce the desired DNA sequence.

Once the Fv variable light and heavy chain DNA is obtained, the sequences may be ligated together, either directly or through a DNA sequence encoding a peptide linker, using techniques well known to those of skill in the art. In one embodiment, heavy and light chain regions are connected by a flexible peptide linker (e.g., (Gly₄Ser)₃) which starts at the carboxyl end of the heavy chain Fv domain and ends at the amino terminus of the light chain Fv domain. The entire sequence encodes the Fv domain in the form of a single-chain antigen binding protein.

### Therapeutic Agents

Compounds defined herein can be used to deliver a therapeutic agent. Examples of therapeutic agents include, but are not limited to, an antigen, a cytotoxic agent, a drug and/or pharmacological agent.

In some embodiments, the therapeutic agent may be a polypeptide fused to the compound. Fusion polypeptides comprising the compound may be prepared by methods known to one of skill in the art. For example, a gene encoding an Fv region is fused to a gene encoding a therapeutic agent. Optionally, the Fv gene is linked to a segment encoding a peptide connector. The peptide connector may be present simply to provide space between the compound and the therapeutic agent or to facilitate mobility between these regions to enable them to each attain their optimum conformation. The DNA sequence comprising the connector may also provide sequences (such as primer sites or restriction sites) to facilitate cloning or may preserve the reading frame between the sequence encoding the binding moiety and the sequence encoding the therapeutic agent. The design of such connector peptides is well known to those of skill in the art.

Generally producing fusion polypeptides involves, e.g., separately preparing the Fv light and heavy chains and DNA encoding any other protein to which they are fused and recombining the DNA sequences in a plasmid or other vector to form a construct encoding the particular desired fusion polypeptide. However, a simpler approach involves inserting the DNA encoding the particular Fv region into a construct already encoding the desired fused polypeptide. The DNA sequence encoding the Fv region is inserted into the construct using techniques well known to those of skill in the art.

Compounds useful for the invention, e.g., recombinant single chain antibodies, may be fused to, or otherwise bound to the therapeutic agent by any method known and available to those in the art. The two components may be chemically bonded together by any of a variety of well-known chemical procedures. For example, the linkage may be by way of heterobifunctional cross-linkers; e.g., SPDP, carbodiimide, glutaraldehyde, or the like. Production of various immunotoxins, as Well as chemical conjugation methods, are well-known within the art (see, for example, “Monoclonal Antibody-Toxin Conjugates: Aiming the Magic Bullet,” Thorpe et al., Monoclonal Antibodies in Clinical Medicine, Academic Press, p. 168-190 (1982); Waldmann, 1991; Vitetta et al., 1987; Pastan et al., 1986; and Thorpe et al., 1987).

Examples of drugs and/or pharmacological agents include, but are not limited to, agents that promote DC activation (e.g. TLR ligands), agents that suppress DC activation or function (e.g. specific inhibitors or promoters of DC signalling molecules such as kinases and phosphatases), and agents that modulate DC death (e.g. promoters or suppressors of apoptosis). Such drugs and/or pharmacological agents are well known to those skilled in the art.

The skilled person will appreciate that there are a number of bacterial or plant polypeptide toxins that are suitable for use as cytotoxic agents in the methods of the invention. These polypeptides include, but are not limited to, polypeptides such as native or modified Pseudomonas exotoxin, diphtheria toxin (DT), ricin, abrin, gelonin, momordin II, bacterial RIPs such as shiga and shiga-like toxin a-chains, luffin, atrichosanthin, momordin I, Mirabilis anti-viral protein, pokeweed antiviral protein, byodin 2 (U.S. Pat. No. 5,597,569), gaporin, as well as genetically engineered variants thereof. Native PE and DT are highly toxic compounds that typically bring about death through liver toxicity. Preferably, Pseudomonas exotoxin and DT are modified into a form that removes the native targeting component of the toxin, e.g., domain Ia of Pseudomonas exotoxin and the B chain of DT. One of skill in the art will appreciate that the invention is not limited to a particular cytotoxic agent.

Other suitable cytotoxic agents for use in the present invention include, but are not limited to, agents such as bacterial or plant toxins, drugs, e.g., cyclophosphamide (CTX; Cytoxan), chlorambucil (CHL; leukeran), cisplatin (Cis P; CDDP; platinol), busulfan (myleran), melphalan, carmustine (BCNU), streptozotocin, triethylenemelamine (TEM), mitomycin C, and other alkylating agents; methotrexate (MTX), etoposide (VP-16; vepesid), 6-mercaptopurine (6 MP), 6-thioguanine (6TG), cytarabine (Ara-C), 5-fluorouracil (5FU), dacarbazine (DTIC), 2-chlorodeoxyadenosine (2-CdA), and other antimetabolites; antibiotics including actinomycin D, doxorubicin (DXR; adriamycin), daunorubicin (daunomycin), bleomycin, mithramycin as well as other antibiotics; alkaloids such as vincristine (VCR), vinblastine, and the like; as well as other anti-cancer agents including the cytostatic agents glucocorticoids such as dexamethasone (DEX; decadron) and corticosteroids such as prednisone, nucleotide enzyme inhibitors such as hydroxyurea, and the like.

Those skilled in the art will realize that there are numerous other radioisotopes and chemocytotoxic agents that can be coupled to compounds of the invention by well known techniques, and delivered to specifically destroy dendritic cells (see, e.g., U.S. Pat. No. 4,542,225). Examples of photo-activated toxins include dihydropyridine- and omega-cynotoxin. Examples of cytotoxic reagents that can be used include ¹²⁵I, ¹³¹I, ¹¹¹In, ¹²³I, ⁹⁹mTc, and ³²P. The antibody can be labeled with such reagents using techniques known in the art. For example, see Wenzel and Meares, Radioimmunoimaging and Radioimmunotherapy, Elsevier, N.Y. (1983) for techniques relating to the radiolabeling of antibodies (see also, Colcher et al., 1986; “Order, Analysis, Results and Future Prospective of the Therapeutic Use of Radiolabeled Antibody in Cancer Therapy”, Monoclonal Antibodies for Cancer Detection and Therapy, Baldwin et al. (editors), Academic Press, p. 303-16, (1985)).

In one example, the linker-chelator tiuexutan is conjugated to the compound by a stable thiourea covalent bond to provide a high-affinity chelation site for Indium-111 or Yttrium-90.

### Antigens

Compounds useful for the methods of the invention may also be conjugated to an “antigen”.

The term “antigen” is further intended to encompass peptide or protein analogs of known or wild-type antigens such as those described above. The analogs may be more soluble or more stable than wild type antigen, and may also contain mutations or modifications rendering the antigen more immunologically active. Also useful in the present invention are peptides or proteins which have amino acid sequences homologous with a desired antigen's amino acid sequence, where the homologous antigen induces an immune response to the respective tumor or organism.

A “cancer antigen,” as used herein is a molecule or compound (e.g., a protein, peptide, polypeptide, lipid, glycolipid, carbohydrate and/or DNA) associated with a tumor or cancer cell and which is capable of provoking an immune response when expressed on the surface of an antigen presenting cell in the context of an MHC molecule. Cancer antigens include self antigens, as well as other antigens that may not be specifically associated with a cancer, but nonetheless induce and/or enhance an immune response to and/or reduce the growth of a tumor or cancer cell when administered to an animal.

An “antigen from a pathogenic and/or infectious organism” as used herein, is an antigen of any organism and includes, but is not limited to, infectious virus, infectious bacteria, infectious parasites including protozoa (such as Plasmodium sp.) and worms and infectious fungi. Typically, for use in the invention the antigen is a protein or antigenic fragment thereof from the organism, or a synthetic compound which is identical to or similar to naturally-occurring antigen which induces an immune response specific for the corresponding organism. Compounds or antigens that are similar to a naturally-occurring organism antigens are well known to those of ordinary skill in the art. A non-limiting example of a compound that is similar to a naturally-occurring organism antigen is a peptide mimic of a polysaccharide antigen.

Specific embodiments of cancer antigens include, e.g., mutated antigens such as the protein products of the Ras p21 protooncogenes, tumor suppressor p53 and HER-2/neu and BCR-abl oncogenes, as well as CDK4, MUM1, Caspase 8, and Beta catenin; overexpressed antigens such as galectin 4, galectin 9, carbonic anhydrase, Aldolase A, PRAME, Her2/neu, ErbB-2 and KSA, oncofetal antigens such as alpha fetoprotein (AFP), human chorionic gonadotropin (hCG); self antigens such as carcinoembryonic antigen (CEA) and melanocyte differentiation antigens such as Mart 1/Melan A, gp100, gp75, Tyrosinase, TRP1 and TRP2; prostate associated antigens such as PSA, PAP, PSMA, PSM-P1 and PSM-P2; reactivated embryonic gene products such as MAGE 1, MAGE 3, MAGE 4, GAGE 1, GAGE 2, BAGE, RAGE, and other cancer testis antigens such as NY-ESO1, SSX2 and SCP1; mucins such as Muc-1 and Muc-2; gangliosides such as GM2, GD2 and GD3, neutral glycolipids and glycoproteins such as Lewis (y) and globo-H; and glycoproteins such as Tn, Thompson-Freidenreich antigen (TF) and sTn.

Cancer antigens and their respective tumor cell targets include, e.g., cytokeratins, particularly cytokeratin 8, 18 and 19, as antigens for carcinoma. Epithelial membrane antigen (EMA), human embryonic antigen (HEA-125), human milk fat globules, MBr1, MBr8, Ber-EP4, 17-1A, C26 and T16 are also known carcinoma antigens. Desmin and muscle-specific actin are antigens of myogenic sarcomas. Placental alkaline phosphatase, beta-human chorionic gonadotropin, and alpha-fetoprotein are antigens of trophoblastic and germ cell tumors. Prostate specific antigen is an antigen of prostatic carcinomas, carcinoembryonic antigen of colon adenocarcinomas. HMB-45 is an antigen of melanomas. In cervical cancer, useful antigens could be encoded by human papilloma virus. Chromagranin-A and synaptophysin are antigens of neuroendocrine and neuroectodermal tumors. Of particular interest are aggressive tumors that form solid tumor masses having necrotic areas.

Antigens derived from pathogens known to predispose to certain cancers may also be advantageously used in the present invention. Pathogens of particular interest for use in the cancer vaccines provided herein include the hepatitis B virus (hepatocellular carcinoma), hepatitis C virus (heptomas), Epstein Barr virus (EBV) (Burkitt lymphoma, nasopharynx cancer, PTLD in immunosuppressed individuals), HTLVL (adult T cell leukemia), oncogenic human papilloma viruses types 16, 18, 33, 45 (adult cervical cancer), and the bacterium Helicobacter pylori (B cell gastric lymphoma). Other medically relevant microorganisms that may serve as antigens in mammals and more particularly humans are described extensively in the literature, e.g., C. G. A Thomas, Medical Microbiology, Bailliere Tindall, (1983).

Exemplary viral pathogens include, but are not limited to, infectious virus that infect mammals, and more particularly humans. Examples of infectious virus include, but are not limited to: Retroviridae (e.g., human immunodeficiency viruses, such as HIV-1 (also referred to as HTLV-III, LAV or HTLV-III/LAV, or HIV-III; and other isolates, such as HIV-LP; Picornaviridae (e.g. polio viruses, hepatitis A virus; enteroviruses, human Coxsackie viruses, rhinoviruses, echoviruses); Calciviridae (e.g. strains that cause gastroenteritis); Togaviridae (e.g. equine encephalitis viruses, rubella viruses); Flaviridae (e.g. dengue viruses, encephalitis viruses, yellow fever viruses); Coronoviridae (e.g. coronaviruses such as the SARS coronavirus); Rhabdoviradae (e.g. vesicular stomatitis viruses, rabies viruses); Filoviridae (e.g. ebola viruses); Paramyxoviridae (e.g. parainfluenza viruses, mumps virus, measles virus, respiratory syncytial virus); Orthomyxoviridae (e.g. influenza viruses); Bungaviridae (e.g. Hantaan viruses, bunga viruses, phleboviruses and Nairo viruses); Arena viridae (hemorrhagic fever viruses); Reoviridae (e.g. reoviruses, orbiviurses and rotaviruses); Birnaviridae; Hepadnaviridae (Hepatitis B virus); Parvovirida (parvoviruses); Papovaviridae (papilloma viruses, polyoma viruses); Adenoviridae (most adenoviruses); Herpesviridae herpes simplex virus (HSV) 1 and 2, varicella zoster virus, cytomegalovirus (CMV), herpes virus; Poxyiridae (variola viruses, vaccinia viruses, pox viruses); and Iridoviridae (e.g. African swine fever virus); and unclassified viruses (e.g. the etiological agents of Spongiform encephalopathies, the agent of delta hepatitis (thought to be a defective satellite of hepatitis B virus), the agents of non-A, non-B hepatitis (class 1=internally transmitted; class 2=parenterally transmitted (i.e. Hepatitis C); Norwalk and related viruses, and astroviruses).

Also, gram negative and gram positive bacteria may be targeted by the subject compositions and methods in vertebrate animals. Such gram positive bacteria include, but are not limited to Pasteurella sp., Staphylococci sp., and Streptococcus sp. Gram negative bacteria include, but are not limited to, Escherichia coli, Pseudomonas sp., and Salmonella sp. Specific examples of infectious bacteria include but are not limited to: Helicobacter pyloris, Borella burgdorferi, Legionella pneumophilia, Mycobacteria sp. (e.g. M. tuberculosis, M avium, M intracellulare, M kansaii, M gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus infuenzae, Bacillus antracis, Corynebacterium diphtheriae, Corynebacterium sp., Erysipelothrix rhusiopathiae, Clostridium perfringens, Clostridium tetani, Enterobacter aerogenes, Klebsiella pneumoniae, Pasturella multocida, Bacteroides sp., Fusobacterium nucleatum, Streptobacillus moniliformis, Treponema pallidium, Treponema pertenue, Leptospira, Rickettsia, and Actinomyces israelli.

Polypeptides of bacterial pathogens which may find use as sources of antigen in the subject compositions include but are not limited to an iron-regulated outer membrane protein, (“IROMP”), an outer membrane protein (“OMP”), and an A-protein of Aeromonis salmonicida which causes furunculosis, p57 protein of Renibacterium salmoninarum which causes bacterial kidney disease (“BIND”), major surface associated antigen (“msa”), a surface expressed cytotoxin (“mpr”), a surface expressed hemolysin (“ish”), and a flagellar antigen of Yersiniosis; an extracellular protein (“ECP”), an iron-regulated outer membrane protein (“IROMP”), and a structural protein of Pasteurellosis; an OMP and a flagellar protein of Vibrosis anguillarum and V. ordalii; a flagellar protein, an OMP protein, aroA, and purA of Edwardsiellosis ictaluri and E. tarda; and surface antigen of Ichthyophthirius; and a structural and regulatory protein of Cytophaga columnari; and a structural and regulatory protein of Rickettsia. Such antigens can be isolated or prepared recombinantly or by any other means known in the art.

Examples of pathogens further include, but are not limited to, infectious fungi and parasites that infect mammals, and more particularly humans. Examples of infectious fungi include, but are not limited to: Cryptococcus neoformans, Histoplasma capsulatum, Coccidioides immitis, Blastomyces dermatitidis, Chlamydia trachomatis, and Candida albicans.

Examples of parasites include intracellular parasites and obligate intracellular parasites. Examples of parasites include but are not limited to Plasmodium falciparum, Plasmodium ovale, Plasmodium malariae, Plasmdodium vivax, Plasmodium knowlesi, Babesia microti, Babesia divergens, Trypanosoma cruzi, Toxoplasma gondii, Trichinella spiralis, Leishmania major, Leishmania donovani, Leishmania braziliensis, Leishmania tropica, Trypanosoma gambiense, Trypanosoma rhodesiense, Wuchereria bancrofti, Brugia malayi, Brugia timori, Ascaris lumbricoides, Onchocerca volvulus and Schistosoma mansoni.

Other medically relevant microorganisms that serve as antigens in mammals and more particularly humans are described extensively in the literature, e.g., see C. G. A Thomas, Medical Microbiology, Bailliere Tindall, (1983). In addition to the treatment of infectious human diseases and human pathogens, the compositions and methods of the present invention are useful for treating infections of nonhuman mammals. Exemplary non-human pathogens include, but are not limited to, mouse mammary tumor virus (“MMTV”), Rous sarcoma virus (“RSV”), avian leukemia virus (“ALV”), avian myeloblastosis virus (“AMV”), murine leukemia virus (“MLV”), feline leukemia virus (“FeLV”), murine sarcoma virus (“MSV”), gibbon ape leukemia virus (“GALV”), spleen necrosis virus (“SNV”), reticuloendotheliosis virus (“RV”), simian sarcoma virus (“SSV”), Mason-Pfizer monkey virus (“MPMV”), simian retrovirus type 1 (“SRV-1”), lentiviruses such as HIV-1, SIV, Visna virus, feline immunodeficiency virus (“FIV”), and equine infectious anemia virus (“EIAV”), T-cell leukemia viruses such as HTLV-1, HTLV-II, simian T-cell leukemia virus (“STLV”), and bovine leukemia virus (“BLV”), and foamy viruses such as human foamy virus (“HFV”), simian foamy virus (“SFV”) and bovine foamy virus (“BFV”).

### Detectable Labels

Compounds useful for the invention may be employed in a range of detection systems. For example, the compound may be used in methods for imaging an internal region of a subject and/or diagnosing the presence or absence of a disease in a subject.

It will be apparent to those skilled in the art that the diagnostic, prognostic and/or monitoring methods of the present invention involve a degree of quantification to determine levels of 5B6, 5B6 expressing cells, ligand and/or ligand expressing cells present in patient samples. Such quantification is readily provided by the inclusion of appropriate control samples.

Preferably, internal controls are included in the methods of the present invention. A preferred internal control is one or more samples taken from one or more healthy individuals.

Compounds useful for the present invention when used diagnostically may be linked to a diagnostic reagent such as a detectable label to allow easy detection of binding events in vitro or in vivo. Suitable labels include radioisotopes, or non-radioactive labels such as biotin, enzymes, chemiluminescent molecules, fluorophores, dye markers or other imaging reagents for detection and/or localisation of target molecules. Alternatively, a second labelled antibody or avidin (for example) which binds the compound can be used for detection.

In the case of an enzyme immunoassay, an enzyme can be conjugated to the second antibody, generally by means of glutaraldehyde or periodate. As will be readily recognized, however, a wide variety of different conjugation techniques exist, which are readily available to the skilled artisan. Commonly used enzymes include horseradish peroxidase, glucose oxidase, β-galactosidase and alkaline phosphatase, amongst others. The substrates to be used with the specific enzymes are generally chosen for the production, upon hydrolysis by the corresponding enzyme, of a detectable color change. Examples of suitable enzymes include alkaline phosphatase and peroxidase. It is also possible to employ fluorogenic substrates, which yield a fluorescent product rather than the chromogenic substrates noted above.

In another example, fluorescent compounds, such, as but not limited to fluorecein and rhodamine amongst others, may be chemically coupled to, for examples, antibodies without altering their binding capacity. When activated by illumination with light of a particular wavelength, the fluorochrome-labelled ‘antibody adsorbs the light energy, inducing a state to excitability in’ the molecule, followed by emission of the light at a characteristic color visually detectable with a light microscope.

By further way of non-limiting example, the compounds coupled to imaging agents can be used in the detection of 5B6 or ligand expression in histochemical tissue sections. The compound may be covalently or non-covalently coupled to a suitable supermagnetic, paramagnetic, electron dense, chogenic, radioactive, or non-radioactive labels such as biotin or avidin.

### Labelled Cell Detection and Isolation

Cells with a disrupted cell membrane, cells infected with a pathogen, dying cells or dead cells can be detected in a sample by a variety of techniques well known in the art, including cell sorting, especially fluorescence-activated cell sorting (FACS), by using an affinity reagent bound to a substrate (e.g., a plastic surface, as in panning), or by using an affinity reagent bound to a solid phase particle which can be isolated on the basis of the properties of the beads (e.g., colored latex beads or magnetic particles). Naturally, the procedure used to detect the cells will depend upon how the cells have been labelled.

In one example, any detectable substance which has the appropriate characteristics for the cell sorter may be used (e.g., in the case of a fluorescent dye, a dye which can be excited by the sorter's light source, and an emission spectra which can be detected by the cell sorter's detectors). In flow cytometry, a beam of laser light is projected through a liquid stream that contains cells, or other particles, which when struck by the focussed light give out signals which are picked up by detectors. These signals are then converted for computer storage and data analysis, and can provide information about various cellular properties. Cells labelled with a suitable dye are excited by the laser beam, and emit light at characteristic wavelengths. This emitted light is picked up by detectors, and these analogue signals are converted to digital signals, allowing for their storage, analysis and display.

Many larger flow cytometers are also “cell sorters”, such as fluorescence-activated cell sorters (FACS), and are instruments which have the ability to selectively deposit cells from particular populations into tubes, or other collection vessels. In a particularly preferred embodiment, the cells are isolated using FACS. This procedure is well known in the art and described by, for example, Melamed et al., Flow Cytometry and Sorting, Wiley-Liss, Inc., (1990); Shapiro, Practical Flow Cytometry, 4th Edition, Wiley-Liss, Inc., (2003); and Robinson et al., Handbook of Flow Cytometry Methods, Wiley-Liss, Inc. (1993).

In order to sort cells, the instruments electronics interprets the signals collected for each cell as it is interrogated by the laser beam and compares the signal with sorting criteria set on the computer. If the cell meets the required criteria, an electrical charge is applied to the liquid stream which is being accurately broken into droplets containing the cells. This charge is applied to the stream at the precise moment the cell of interest is about to break off from the stream, then removed when the charged droplet has broken from the stream. As the droplets fall, they pass between two metal plates, which are strongly positively or negatively charged. Charged droplets get drawn towards the metal plate of the opposite polarity, and deposited in the collection vessel, or onto a microscope slide, for further examination.

The cells can automatically be deposited in collection vessels as single cells or as a plurality of cells, e.g. using a laser, e.g. an argon laser (488 nm) and for example with a Flow Cytometer fitted with an Autoclone unit (Coulter EPICS Altra, Beckman-Coulter, Miami, Fla., USA). Other examples of suitable FACS machines useful for the methods of the invention include, but are not limited to, MoFlo™ High-speed cell sorter (Dako-Cytomation ltd), FACS Aria™ (Becton Dickinson), FACS Diva (Becton Dickinson), ALTRA™ Hyper sort (Beckman Coulter) and CyFlow™ sorting system (Partec GmbH).

For the detection of cells with a disrupted cell membrane, cells infected with a pathogen, dying cells or dead cells from a sample using solid-phase particles, any particle with the desired properties may be utilized. For example, large particles (e.g., greater than about 90-100 μm in diameter) may be used to facilitate sedimentation. Preferably, the particles are “magnetic particles” (i.e., particles which can be collected using a magnetic field). Labelled cells are retained in the column (held by the magnetic field), whilst unlabelled cells pass straight through and are eluted at the other end. Magnetic particles are now commonly available from a variety of manufacturers including Dynal Biotech (Oslo, Norway) and Milteni Biotech GmbH (Germany). An example of magnetic cell sorting (MACS) is provided by Al-Mufti et al. (1999).

Laser-capture microdissection can also be used to selectively detect labelled cells on a slide using methods of the invention. Methods of using laser-capture microdissection are known, in the art (see, for example, U.S. 20030227611 and Bauer et al., 2002).

### Labelled Dendritic Cell or Precursor Thereof Detection and Isolation

As used herein, the terms “enriching” and “enriched” are used in their broadest sense to encompass the isolation of dendritic cells or precursors thereof such that the relative concentration of dendritic cells or precursors thereof to non-dendritic cells or precursors thereof in the treated sample is greater than a comparable untreated sample. Preferably, the enriched dendritic cells and/or precursors thereof are separated from at least 10%, more preferably at least 20%, more preferably at least 30%, more preferably at least 40%, more preferably at least 50%, more preferably at least 60%, more preferably at least 70%, more preferably at least 75%, more preferably at least 80%, more preferably at least 90%, more preferably at least 95%, and even more preferably at least 99% of the non-dendritic cells or precursors thereof in the sample obtained from the original sample. Most preferably, the enriched cell population contains no non-dendritic cells or precursors thereof (namely, pure). The terms “enrich” and variations thereof are used interchangeably herein with the term “isolate” and variations thereof. Furthermore, a population of cells enriched using a method of the invention may only comprise a single dendritic cell or precursor thereof. In addition, the enrichment methods of the invention may be used to isolate a single dendritic cell or precursor thereof.

Dendritic cells or precursors thereof can be enriched from the sample by a variety of techniques well known in the art, including cell sorting, especially fluorescence-activated cell sorting (FACS), by using an affinity reagent bound to a substrate (e.g., a plastic surface, as in panning), or by using an affinity reagent bound to a solid phase particle which can be isolated on the basis of the properties of the beads (e.g., colored latex beads or magnetic particles). Naturally, the procedure used to enrich the dendritic cells and/or precursors thereof will depend upon how the cells have been labelled.

In one example, any detectable substance which has the appropriate characteristics for the cell sorter may be used (e.g., in the case of a fluorescent dye, a dye which can be excited by the sorter's light source, and an emission spectra which can be detected by the cell sorter's detectors). In flow cytometry, a beam of laser light is projected through a liquid stream that contains cells, or other particles, which when struck by the focussed light give out signals which are picked up by detectors. These signals are then converted for computer storage and data analysis, and can provide information about various cellular properties. Cells labelled with a suitable dye are excited by the laser beam, and emit light at characteristic wavelengths. This emitted light is picked up by detectors, and these analogue signals are converted to digital signals, allowing for their storage, analysis and display.

Many larger flow cytometers are also “cell sorters”, such as fluorescence-activated cell sorters (FACS), and are instruments which have the ability to selectively deposit cells from particular populations into tubes, or other collection vessels. In a particularly preferred embodiment, the cells are isolated using FACS. This procedure is well known in the art and described by, for example, Melamed et al., Flow Cytometry and Sorting, Wiley-Liss, Inc., (1990); Shapiro, Practical Flow Cytometry, 4th Edition, Wiley-Liss, Inc., (2003); and Robinson et al., Handbook of Flow Cytometry Methods, Wiley-Liss, Inc. (1993).

In order to sort cells, the instruments electronics interprets the signals collected for each cell as it is interrogated by the laser beam and compares the signal with sorting criteria set on the computer. If the cell meets the required criteria, an electrical charge is applied to the liquid stream which is being accurately broken into droplets containing the cells. This charge is applied to the stream at the precise moment the cell of interest is about to break off from the stream, then removed when the charged droplet has broken from the stream. As the droplets fall, they pass between two metal plates, which are strongly positively or negatively charged. Charged droplets get drawn towards the metal plate of the opposite polarity, and deposited in the collection vessel, or onto a microscope slide, for further examination.

The cells can automatically be deposited in collection vessels as single cells or as a plurality of cells, e.g. using a laser, e.g. an argon laser (488 nm) and for example with a Flow Cytometer fitted with an Autoclone unit (Coulter EPICS Altra, Beckman-Coulter, Miami, Fla., USA). Other examples of suitable FACS machines useful for the methods of the invention include, but are not limited to, MoFlo™ High-speed cell sorter (Dako-Cytomation ltd), FACS Aria™ (Becton Dickinson), FACS Diva (Becton Dickinson), ALTRA™ Hyper sort (Beckman Coulter) and CyFlow™ sorting system (Partec GmbH).

The enrichment of dendritic cells and/or or precursors thereof from a sample using solid-phase particles, any particle with the desired properties may be utilized. For example, large particles (e.g., greater than about 90-100 μm in diameter) may be used to facilitate sedimentation. Preferably, the particles are “magnetic particles” (i.e., particles which can be collected using a magnetic field). Labelled cells are retained in the column (held by the magnetic field), whilst unlabelled cells pass straight through and are eluted at the other end. Magnetic particles are now commonly available from a variety of manufacturers including Dynal Biotech (Oslo, Norway) and Milteni Biotech GmbH (Germany). An example of magnetic cell sorting (MACS) is provided by Al-Mufti et al. (1999).

Laser-capture microdissection can also be used to selectively enrich labelled dendritic cells or precursors thereof on a slide using methods of the invention. Methods of using laser-capture microdissection are known in the art (see, for example, U.S. 20030227611 and Bauer et al., 2002).

Following enrichment, the cells can be used immediately or cultured in vitro to expand dendritic cells and/or precursors thereof numbers using techniques known in the art. Furthermore, dendritic cell precursors can be cultured to produce Mature dendritic cells.

Identification of Compounds that Bind 5B6 Ligands

Methods of screening test compounds are described which can identify a compound that binds to 5B6 ligands such as spectrin or RNF41, and are thus useful in a method of the invention.

Inhibitors of 5B6 ligand activity are screened by resort to assays and techniques useful in identifying drugs capable of binding to the ligand and thereby inhibiting its biological activity. Such assays include the use of mammalian cell lines (for example, CHO cells or 293T cells) for phage display system for expressing the ligand and using a culture of transfected mammalian or E. coli or other microorganism to produce the proteins for binding studies of potential binding compounds.

As another example, a method for identifying compounds which specifically bind to a 5B6 ligand can include simply the steps of contacting a selected cell expressing the ligand with a test compound to permit binding of the test compound to the ligand, and determining the amount of test compound, if any, which is bound to the ligand. Such a method involves the incubation of the test compound and the ligand immobilized on a solid support. Typically, the surface containing the immobilized compound is permitted to come into contact with a solution containing the protein and binding is measured using an appropriate detection system. Suitable detection systems are known in the art, some of which are described herein.

Computer modeling and searching technologies permit identification of compounds that can bind 5B6 ligand. The three dimensional geometric structure of the 5B6 ligand, or the active site thereof can be determined. This can be done by known methods, including X-ray crystallography, which can determine a complete molecular structure.

Methods of computer based numerical modeling can be used to complete the structure (e.g., in embodiments wherein an incomplete or insufficiently accurate structure is determined) or to improve its accuracy. Any method recognized in the art may be used, including, but not limited to, parameterized models specific to particular biopolymers such as proteins or nucleic acids, molecular dynamics models based on computing molecular motions, statistical mechanics models based on thermal ensembles, or combined models.

The three-dimensional structure of a 5B6 ligand can be used to identify antagonists or agonists through the use of computer modeling using a docking program such as GRAM, DOCK, or AUTODOCK (Dunbrack et al., 1997). Computer programs can also be employed to estimate the attraction, repulsion, and steric hindrance of a candidate compound to the polypeptide. Generally the tighter the fit (e.g., the lower the steric hindrance, and/or the greater the attractive force) the more potent the potential agonist or antagonist will be since these properties are consistent with a tighter binding constant. Furthermore, the more specificity in the design of a potential agonist or antagonist the more likely that it will not interfere with other proteins.

Initially a potential compound could be obtained, for example, by screening a random peptide library produced by a recombinant bacteriophage or a chemical library. A compound selected in this manner could be then be systematically modified by computer modeling programs until one or more promising potential compounds are identified.

Such computer modeling allows the selection of a finite number of rational chemical modifications, as opposed to the countless number of essentially random chemical modifications that could be made, and of which any one might lead to a useful agonist or antagonist. Each chemical modification requires additional chemical steps, which while being reasonable for the synthesis of a finite number of compounds, quickly becomes overwhelming if all possible modifications needed to be synthesized. Thus through the use of the three-dimensional structure and computer modeling, a large number of these compounds can be rapidly screened on the computer monitor screen, and a few likely candidates can be determined without the laborious synthesis of untold numbers of compounds.

For most types of models, standard molecular force fields, representing the forces between constituent atoms and groups, are necessary, and can be selected from force fields known in physical chemistry. Exemplary forcefields that are known in the art and can be used in such methods include, but are not limited to, the Constant Valence Force Field (CVFF), the AMBER force field and the CHARM force field. The incomplete or less accurate experimental structures can serve as constraints on the complete and more accurate structures computed by these modeling methods.

Further examples of molecular modeling systems are the CHARMm and QUANTA programs (Polygen Corporation, Waltham, Mass.). CHARMm performs the energy minimization and molecular dynamics functions. QUANTA performs the construction, graphic modelling and analysis of molecular structure. QUANTA allows interactive construction, modification, visualization, and analysis of the behaviour of molecules with each other.

Diseases Associated with Cells with a Disrupted Cell Membrane, Cells Infected with a Pathogen, Dying Cells or Dead Cells

Examples of the diseases associated with cells with a disrupted cell membrane, cells infected with a pathogen, dying cells or dead cells include, but are not necessarily limited to, the following:

1) Diseases in which apoptosis is induced in response to a signal generated by a cell of an immune system responsible for biophylaxis, for example, graft versus host disease (GVHD) and autoimmune diseases (systemic lupus erythematosus (SLE), rheumatoid arthritis (RA), scleroderma, Sjogren's syndrome, multiple sclerosis, insulin dependent diabetes mellitus, ulcerative colitis).

2) Diseases in which cell death is induced by viral infection or apoptosis is induced by reaction of a cell of an immune system with a cell infected by a virus or parasitic infection, for example, virus associated hemophagocytic syndrome (VAHS) and other viral infections (HCV, HIV, influenza virus).

3) Diseases in which cell death is induced by an abnormal apoptosis signal, for example, neurodegenerative diseases (Alzheimer's disease, Parkinson's disease).

4) Leukemia, for example, acute lymphatic leukemia.

5) Diseases in which apoptosis is artificially induced by, for example, radiation exposure or medication (anticancer drug etc.)

6) Systemic inflammatory reaction syndrome (SIRS), diseases in which organ disorder occurs because the immune system is nonspecifically activated in response to invasion to a living body and thus control of cytokine production becomes impossible (HPS, severe pancreatitis).

7) Diseases where there is a lack of cell death such as cancer.

8) Injury, particularly post-injury recovery.

Cell death progressing in a living body can be determined using the present invention, and hence progress of these diseases can be monitored. In particular, the invention is useful for GVHD, human immunodeficiency virus (HIV), hemophagocytic syndrome (HPS), especially virus associated hemophagocytic syndrome (VAHS), acute lymphatic leukemia, influenza encephalitis, encephalopathy, and malaria.

### Polypeptides

The terms “polypeptide” and “protein” are generally used interchangeably and refer to a single polypeptide chain which may or may not be modified by addition of non-amino acid groups. It would be understood that such polypeptide chains may associate with other polypeptides or proteins or other molecules such as co-factors. The terms “proteins” and “polypeptides” as used herein also include variants, mutants, biologically active fragments, modifications, analogous and/or derivatives of the polypeptides described herein.

The % identity of a polypeptide is determined by GAP (Needleman and Wunsch, 1970) analysis (GCG program) with a gap creation penalty=5, and a gap extension penalty=0.3. The query sequence is at least 25 amino acids in length, and the GAP analysis aligns the two sequences over a region of at least 25 amino acids. More preferably, the query sequence is at least 50 amino acids in length, and the GAP analysis aligns the two sequences over a region of at least 50 amino acids. More preferably, the query sequence is at least 100 amino acids in length and the GAP analysis aligns the two sequences over a region of at least 100 amino acids. Even more preferably, the query sequence is at least 200 amino acids in length and the GAP analysis aligns the two sequences over a region of at least 200 amino acids. Even more preferably, the GAP analysis aligns the two sequences over their entire length.

As used herein a “biologically active fragment” is a portion of a polypeptide as described herein which maintains a defined activity of the full-length polypeptide. Biologically active fragments can be any size as long as they maintain the defined activity. Preferably, biologically active fragments are at least 100 amino acids in length. With regard to ligands of 5B6 such as spectrin and RN41, a preferred biological activity is the binding to Clec9A.

In an embodiment, a second polypeptide comprising an amino acid sequence which is at least 50% identical to any one or more of SEQ ID NO's 1 to 8 is, or comprises, a biologically active and/or soluble fragment of one of SEQ ID NO's 1 to 8. As used herein, a “soluble fragment” refers to a portion of a polypeptide which is lacking a membrane spanning region. In a preferred embodiment, the soluble fragment does not comprise at least the about 40, at least about 50, at least about 55, or at least about 100, N-terminal residues of any one of SEQ ID NO's 1 to 8. In a further preferred embodiment, the soluble fragment comprises the C-type lectin-like domain of a polypeptide which comprises:

i) an amino acid sequence as provided in any one of SEQ ID NO's 1 to 8; or

ii) an amino acid sequence which is at least 50% identical to any one or more of SEQ ID NO's 1 to 8. In a further embodiment, the soluble fragment comprises:

i) an amino acid sequence as provided in any one of SEQ ID NO's 40 to 47; or ii) an amino acid sequence which is at least 50% identical to any one or more of SEQ ID NO's 40 to 47,

wherein the soluble fragment does not comprise at least the about 40 N-terminal residues of anyone of SEQ ID NO's 1 to 8.

With regard to a defined polypeptide, it will be appreciated that % identity figures higher than those provided above will encompass preferred embodiments. Thus, where applicable, in light of the minimum % identity figures, it is preferred that the polypeptide comprises an amino acid sequence which is at least 50%, more preferably at least 55%, more preferably at least 60%, more preferably at least 65%, more preferably at least 70%, more preferably at least 75%, more preferably at least 80%, more preferably at least 85%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, more preferably at least 99%, more preferably at least 99.1%, more preferably at least 99.2%, more preferably at least 99.3%, more preferably at least 99.4%, more preferably at least 99.5%, more preferably at least 99.6%, more preferably at least 99.7%, more preferably at least 99.8%, and even more preferably at least 99.9% identical to the relevant nominated SEQ ID NO.

Amino acid sequence mutants of a polypeptide described herein can be prepared by introducing appropriate nucleotide changes into a nucleic acid defined herein, or by in vitro synthesis of the desired polypeptide. Such mutants include, for example, deletions, insertions or substitutions of residues within the amino acid sequence. A combination of deletion, insertion and substitution can be made to arrive at the final construct, provided that the final polypeptide product possesses the desired characteristics.

Mutant (altered) polypeptides can be prepared using any technique known in the art. For example, a polynucleotide described herein can be subjected to in vitro mutagenesis. Such in vitro mutagenesis techniques may include sub-cloning the polynucleotide into a suitable vector, transforming the vector into a “mutator” strain such as the E. coli XL-1 red (Stratagene) and propagating the transformed bacteria for a suitable number of generations. In another example, the polynucleotides defined herein are subjected to DNA shuffling techniques as broadly described by Harayama (1998). Products derived from mutated/altered DNA can readily be screened using techniques described herein to determine if they are able to confer the desired phenotype.

In designing amino acid sequence mutants, the location of the mutation site and the nature of the mutation will depend on characteristic(s) to be modified. The sites for mutation can be modified individually or in series, e.g., by (1) substituting first with conservative amino acid choices and then with more radical selections depending upon the results achieved, (2) deleting the target residue, or (3) inserting other residues adjacent to the located site.

Amino acid sequence deletions generally range from about 1 to 15 residues, more preferably about 1 to 10 residues and typically about 1 to 5 contiguous residues.

Substitution mutants have at least one amino acid residue in the polypeptide molecule removed and a different residue inserted in its place. The sites of greatest interest for substitutional mutagenesis include sites identified as important for function. Other sites of interest are those in which particular residues obtained from various strains or species are identical, and/or those in which particular residues obtained from related proteins are identical. These positions may be important for biological activity. These sites, especially those falling within a sequence of at least three other identically conserved sites, are preferably substituted in a relatively conservative manner. Such conservative substitutions are shown in Table 1.

TABLE 1
Exemplary substitutions.

| Original | Exemplary |
|---|---|
| Residue | Substitutions |
| Ala (A) | val; leu; ile; gly |
| Arg (R) | lys |
| Asn (N) | gln; his |
| Asp (D) | glu |
| Cys (C) | ser |
| Gln (Q) | asn; his |
| Glu (E) | asp |
| Gly (G) | pro, ala |
| His (H) | asn; gln |
| Ile (I) | leu; val; ala |
| Leu (L) | ile; val; met; ala; phe |
| Lys (K) | arg |
| Met (M) | leu; phe |
| Phe (F) | leu; val; ala |
| Pro (P) | gly |
| Ser (S) | thr |
| Thr (T) | ser |
| Trp (W) | tyr |
| Tyr (Y) | trp; phe |
| Val (V) | ile; leu; met; phe; ala |

Furthermore, if desired, unnatural amino acids or chemical amino acid analogues can be introduced as a substitution or addition into a polypeptides described herein. Such amino acids include, but are not limited to, the D-isomers of the common amino acids, 2,4-diaminobutyric acid, α-amino isobutyric acid, 4-aminobutyric acid, 2-aminobutyric acid, 6-amino hexanoic acid, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, β-alanine, fluoro-amino acids, designer amino acids such as β-methyl amino acids, Cα-methyl amino acids, Nα-methyl amino acids, and amino acid analogues in general.

Also included within the scope of the invention are the use of polypeptides which are differentially modified during or after synthesis, e.g., by biotinylation, benzylation, glycosylation, acetylation, phosphorylation; amidation, derivatization by known protecting/blocking groups, proteolytic cleavage, linkage to an antibody molecule or other cellular ligand, etc. These modifications may serve to increase the stability and/or bioactivity of the polypeptide.

Polypeptides described herein can be produced in a variety of ways, including production and recovery of natural polypeptides, production and recovery of recombinant polypeptides, and chemical synthesis of the polypeptides. In one embodiment, an isolated polypeptide of the present invention is produced by culturing a cell capable of expressing the polypeptide under conditions effective to produce the polypeptide, and recovering the polypeptide. A preferred cell to culture is a recombinant cell of the present invention. Effective culture conditions include, but are not limited to, effective media, bioreactor, temperature, pH and oxygen conditions that permit polypeptide production. An effective medium refers to any medium in which a cell is cultured to produce a polypeptide of the present invention. Such medium typically comprises an aqueous medium having assailable carbon, nitrogen and phosphate sources, and appropriate salts, minerals, metals and other nutrients, such as vitamins. Cells of the present invention can be cultured in conventional fermentation bioreactors, tissue culture flasks, shake flasks, test tubes, microtiter dishes, and petri plates. Culturing can be carried out at a temperature, pH and oxygen content appropriate for a recombinant cell. Such culturing conditions are within the expertise of one of ordinary skill in the art.

### Polynucleotides

The term “polynucleotide” is used interchangeably herein with the term “nucleic acid”.

The % identity of a polynucleotide is determined by GAP (Needleman and Wunsch, 1970) analysis (GCG program) with a gap creation penalty=5, and a gap extension penalty=0.3. Unless stated otherwise, the query sequence is at least 45 nucleotides in length, and the GAP analysis aligns the two sequences over a region of at least 45 nucleotides. Preferably, the query sequence is at least 150 nucleotides in length, and the GAP analysis aligns the two sequences over a region of at least 150 nucleotides. More preferably, the query sequence is at least 250 nucleotides in length and the GAP analysis aligns the two sequences over a region of at least 250 nucleotides. Even more preferably, the GAP analysis aligns the two sequences over their entire length.

With regard to the defined polynucleotides, it will be appreciated that % identity figures higher than those provided above will encompass preferred embodiments. Thus, where applicable, in light of the minimum % identity figures, it is preferred that a polynucleotide defined herein comprises a sequence which is at least 50%, more preferably at least 55%, more preferably at least 60%, more preferably at least 65%, more preferably at least 70%, more preferably at least 75%, more preferably at least 80%, more preferably at least 85%, more preferably at least 90%, more preferably at least. 91%, more preferably at least 92%, more preferably at least 93%, more preferably at least 94%, more preferably at least 95%, more preferably at least 96%, more preferably at least 97%, more preferably at least 98%, more preferably at least 99%, more preferably at least 99.1%, more preferably at least 99.2%, more preferably at least 99.3%, more preferably at least 99.4%, more preferably at least 99.5%, more preferably at least 99.6%, more preferably at least 99.7%, more preferably at least 99.8%, and even more preferably at least 99.9% identical to the relevant nominated SEQ ID NO.

As used herein, the term “hybridizes” refers to the ability of two single stranded nucleic acid molecules being able to form at least a partially double stranded nucleic acid through hydrogen bonding.

As used herein, the phrase “stringent conditions” refers to conditions under which a polynucleotide, probe, primer and/or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides.

Stringent conditions may also be achieved with the addition of destabilizing agents, such as formamide. Stringent conditions are known to those skilled in the art and can be found in Ausubel et al. (supra), 6.3.1-6.3.6, as well as the Examples described herein. Preferably, the conditions are such that sequences at least about 65%, 70%, 75%, 85%, 90%, 95%, 98%, or 99% homologous to each other typically remain hybridized to each other. A non-limiting example of stringent hybridization conditions are hybridization in a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2.xSSC, 0.01% BSA at 50° C. In another embodiment, a nucleic acid sequence that is hybridizable to one or more of the nucleic acid molecule comprising the nucleotide sequence of SEQ ID NO's 81 to 113, under conditions of moderate stringency is provided. A non-limiting example of moderate stringency hybridization conditions are hybridization in 6×SSC, 5×Denhardt's solution, 0.5% SDS and 100 mg/ml denatured salmon sperm DNA at 55° C., followed by one or more washes in 1×SSC, 0.1% SDS at 37° C. Other conditions of moderate stringency that may be used are well-known within the art, see, e.g., Ausubel et al. (supra), and Kriegler, Gene Transfer and Expression, A Laboratory Manual, Stockton Press, (1990). In yet another embodiment, a nucleic acid that is hybridizable to the nucleic acid molecule comprising any one or more of the nucleotide sequences of SEQ ID NO's 81 to 113, under conditions of low stringency, is provided. A non-limiting example of low stringency hybridization conditions are hybridization in 35% formamide, 5×SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris-HCl (pH 7.4), 5 mM EDTA, and 0.1% SDS at 50° C. Other conditions of low stringency that may be used are well known in the art, see, e.g., Ausubel et al. (supra) and Kriegler (supra).

Polynucleotides may possess, when compared to naturally occurring molecules, one or more mutations which are deletions, insertions, or substitutions of nucleotide residues. Mutants can be either naturally occurring (that is to say, isolated from a natural source) or synthetic (for example, by performing site-directed mutagenesis on the nucleic acid).

Usually, monomers of a polynucleotide or oligonucleotide are linked by phosphodiester bonds or analogs thereof to form oligonucleotides ranging in size from a relatively short monomeric units, e.g., 12-18, to several hundreds of monomeric units. Analogs of phosphodiester linkages include: phosphorothioate, phosphorodithioate, phosphoroselenoate, phosphorodiselenoate, phosphoroanilothioate, phosphoranilidate and phosphoramidate.

### Antisense Polynucleotides

The term “antisense polynucleotide” shall be taken to mean a DNA or RNA, or combination thereof, molecule that is complementary to at least a portion of a specific mRNA molecule encoding a polypeptide and capable of interfering with a post-transcriptional event such as mRNA translation. The use of antisense methods is well known in the art (see for example, G. Hartmann and S. Endres, Manual of Antisense Methodology, Kluwer (1999)). The use of antisense techniques in plants has been reviewed by Bourque, 1995 and Senior, 1998. Bourque, 1995 lists a large number of examples of how antisense sequences have been utilized in plant systems as a method of gene inactivation. She also states that attaining 100% inhibition of any enzyme activity may not be necessary as partial inhibition will more than likely result in measurable change in the system. Senior (1998) states that antisense methods are now a very well established technique for manipulating gene expression.

An antisense polynucleotide useful for the invention will hybridize to a target polynucleotide under physiological conditions. As used herein, the term “an antisense, polynucleotide which hybridises under physiological conditions” means that the polynucleotide (which is fully or partially single stranded) is at least capable of forming a double stranded polynucleotide with mRNA encoding a protein, such as those provided in any one of SEQ ID NO's 81 to 113 under normal conditions in a cell, preferably a human cell.

Antisense molecules may include sequences that correspond to the structural genes or for sequences that effect control over the gene expression or splicing event. For example, the antisense sequence may correspond to the targeted coding region of the target gene, or the 5′-untranslated region (UTR) or the 3′-UTR or combination of these. It may be complementary in part to intron sequences, which may be spliced out during or after transcription, preferably only to exon sequences of the target gene. In view of the generally greater divergence of the UTRs, targeting these regions provides greater specificity of gene inhibition.

The length of the antisense sequence should be at least 19 contiguous nucleotides, preferably at least 50 nucleotides, and more preferably at least 100, 200, 500 or 1000 nucleotides. The full-length sequence complementary to the entire gene transcript may be used. The length is most preferably 100-2000 nucleotides. The degree of identity of the antisense sequence to the targeted transcript should be at least 90% and more preferably 95-100%. The antisense RNA molecule may of course comprise unrelated sequences which may function to stabilize the molecule.

### Catalytic Polynucleotides

The term catalytic polynucleotide/nucleic acid refers to a DNA molecule or DNA-containing molecule (also known in the art as a “deoxyribozyme”) or an RNA or RNA-containing molecule (also known as a “ribozyme”) which specifically recognizes a distinct substrate and catalyzes the chemical modification of this substrate. The nucleic acid bases in the catalytic nucleic acid can be bases A, C, G, T (and U for RNA).

Typically, the catalytic nucleic acid contains an antisense sequence for specific recognition of a target nucleic acid, and a nucleic acid cleaving enzymatic activity (also referred to herein as the “catalytic domain”). The types of ribozymes that are particularly useful in this invention are the hammerhead ribozyme (Haseloff and Gerlach, 1988; Perriman et al., 1992) and the hairpin ribozyme (Shippy et al., 1999).

The ribozymes useful for this invention and DNA encoding the ribozymes can be chemically synthesized using methods well known in the art. The ribozymes can also be prepared from a DNA molecule (that upon transcription, yields an RNA molecule) operably linked to an RNA polymerase promoter, e.g., the promoter for T7 RNA polymerase or SP6 RNA polymerase. When the vector also contains an RNA polymerase promoter operably linked to the DNA molecule, the ribozyme can be produced in vitro upon incubation with RNA polymerase and nucleotides. In a separate embodiment, the DNA can be inserted into an expression cassette or transcription cassette. After synthesis, the RNA molecule can be modified by ligation to a DNA molecule having the ability to stabilize the ribozyme and make it resistant to RNase.

As with antisense polynucleotides described herein, catalytic polynucleotides useful for the invention should also be capable of hybridizing a target nucleic acid molecule (for example an mRNA encoding any polypeptide provided in SEQ ID NO's 81 to 113) under “physiological conditions”, namely those conditions within a cell (especially conditions in an animal cell such as a human cell).

### RNA Interference

RNA interference (RNAi) is particularly useful for specifically inhibiting the production of a particular protein. Although not wishing to be limited by theory, Waterhouse et al. (1998) have provided a model for the mechanism by which dsRNA (duplex RNA) can be used to reduce protein production. This technology relies on the presence of dsRNA molecules that contain a sequence that is essentially identical to the mRNA of the gene of interest or part thereof, in this case an mRNA encoding a polypeptide according to the invention. Conveniently, the dsRNA can be produced from a single promoter in a recombinant vector or host cell, where the sense and anti-sense sequences are flanked by an unrelated sequence which enables the sense and anti-sense sequences to hybridize to form the dsRNA molecule with the unrelated sequence forming a loop structure. The design and production of suitable dsRNA molecules for the present invention is well within the capacity of a person skilled in the art, particularly considering Waterhouse et al. (1998), Smith et al. (2000), WO 99/32619, WO 99/53050, WO 99/49029, and WO 01/34815.

In one example, a DNA is introduced that directs the synthesis of an at least partly double stranded RNA product(s) with homology to the target gene to be inactivated. The DNA therefore comprises both sense and antisense sequences that, when transcribed into RNA, can hybridize to form the double-stranded RNA region. In a preferred embodiment, the sense and antisense sequences are separated by a spacer region that comprises an intron which, when transcribed into RNA, is spliced out. This arrangement has been shown to result in a higher efficiency of gene silencing. The double-stranded region may comprise one or two RNA molecules, transcribed from either one DNA region or two. The presence of the double stranded molecule is thought to trigger a response from an endogenous plant system that destroys both the double stranded RNA and also the homologous RNA transcript from the target plant gene, efficiently reducing or eliminating the activity of the target gene.

The length of the sense and antisense sequences that hybridise should each be at least 19 contiguous nucleotides, preferably at least 30 or 50 nucleotides, and more preferably at least 100, 200, 500 or 1000 nucleotides. The full-length sequence corresponding to the entire gene transcript may be used. The lengths are most preferably 100-2000 nucleotides. The degree of identity of the sense and antisense sequences to the targeted transcript should be at least 85%, preferably at least 90% and more preferably 95-100%. The RNA molecule may of course comprise unrelated sequences which may function to stabilize the molecule. The RNA molecule may be expressed under the control of a RNA polymerase II or RNA polymerase III promoter. Examples of the latter include tRNA or snRNA promoters.

Preferred small interfering RNA (‘siRNA’) molecules comprise a nucleotide sequence that is identical to about 19-21 contiguous nucleotides of the target mRNA. Preferably, the target mRNA sequence commences with the dinucleotide AA, comprises a GC-content of about 30-70% (preferably, 30-60%, more preferably 40-60% and more preferably about 45%-55%), and does not have a high percentage identity to any nucleotide sequence other than the target in the genome of the animal (preferably human) in which it is to be introduced, e.g., as determined by standard BLAST search.

microRNA

MicroRNA regulation is a clearly specialized branch of the RNA silencing pathway that evolved towards gene regulation, diverging from conventional RNAi/PTGS. MicroRNAs are a specific class of small RNAs that are encoded in gene-like elements organized in a characteristic inverted repeat. When transcribed, microRNA genes give rise to stem-looped precursor RNAs from which the microRNAs are subsequently processed. MicroRNAs are typically about 21 nucleotides in length. The released miRNAs are incorporated into RISC-like complexes containing a particular subset of Argonaute proteins that exert sequence-specific gene repression (see, for example, Millar and Waterhouse, 2005; Pasquinelli et al., 2005; Almeida and Allshire, 2005).

### Cosuppression

Another molecular biological approach that may be used is co-suppression. The mechanism of co-suppression is not well understood but is thought to involve post-transcriptional gene silencing (PTGS) and in that regard may be very similar to many examples of antisense suppression. It involves introducing an extra copy of a gene or a fragment thereof into a plant in the sense orientation with respect to a promoter for its expression. The size of the sense fragment, its correspondence to target gene regions, and its degree of sequence identity to the target gene are as for the antisense sequences described above. In some instances the additional copy of the gene sequence interferes with the expression of the target plant gene. Reference is made to WO 97/20936 and EP 0465572 for methods of implementing co-suppression approaches.

### Recombinant Vectors

Recombinant vectors useful for the invention can include at least one polynucleotide molecule described herein, and/or a polynucleotide encoding a polypeptide as described herein, inserted into any vector capable of delivering the polynucleotide molecule into a host cell. Such a vector contains heterologous polynucleotide sequences, that is polynucleotide sequences that are not naturally found adjacent to polynucleotide molecules of the present invention and that preferably are derived from a species other than the species from which the polynucleotide molecule(s) are derived. The vector can be either RNA or DNA, either prokaryotic or eukaryotic, and typically is a transposon (such as described in U.S. Pat. No. 5,792,294), a virus or a plasmid.

One type of recombinant vector comprises the polynucleotide(s) operably linked to an expression vector. The phrase operably linked refers to insertion of a polynucleotide molecule into an expression vector in a manner such that the molecule is able to be expressed when transformed into a host cell. As used herein, an expression vector is a DNA or RNA vector that is capable of transforming a host cell and of effecting expression of a specified polynucleotide molecule. Preferably, the expression vector is also capable of replicating within the host cell. Expression vectors can be either prokaryotic or eukaryotic, and are typically viruses or plasmids. Expression vectors include any vectors that function (i.e., direct gene expression) in recombinant cells, including in bacterial, fungal, endoparasite, arthropod, animal, and plant cells. Vectors can also be used to produce the polypeptide in a cell-free expression system, such systems are well known in the art.

“Operably linked” as used herein refers to a functional relationship between two or more nucleic acid (e.g., DNA) segments. Typically, it refers to the functional relationship of transcriptional regulatory element to a transcribed sequence. For example, a promoter is operably linked to a coding sequence, such as a polynucleotide defined herein, if it stimulates or modulates the transcription of the coding sequence in an appropriate host cell and/or in a cell-free expression system. Generally, promoter transcriptional regulatory elements that are operably linked to a transcribed sequence are physically contiguous to the transcribed sequence, i.e., they are cis-acting. However, some transcriptional regulatory elements, such as enhancers, need not be physically contiguous or located in close proximity to the coding sequences whose transcription they enhance.

In particular, expression vectors contain regulatory sequences such as transcription control sequences, translation control sequences, origins of replication, and other regulatory sequences that are compatible with the recombinant cell and that control the expression of polynucleotide molecules of the present invention. In particular, recombinant molecules of the present invention include transcription control sequences. Transcription control sequences are sequences which control the initiation, elongation, and termination of transcription. Particularly important transcription control sequences are those which control transcription initiation, such as promoter, enhancer, operator and repressor sequences. Suitable transcription control sequences include any transcription control sequence that can function in at least one of the recombinant cells of the present invention. A variety of such transcription control sequences are known to those skilled in the art. Preferred transcription control sequences include those which function in bacterial, yeast, arthropod, nematode, plant or animal cells, such as, but not limited to, tac, lac, trp, trc, oxy-pro, omp/lpp, rrnB, bacteriophage lambda, bacteriophage T7, T7lac; bacteriophage T3, bacteriophage SP6, bacteriophage SP01, metallothionein, alpha-mating factor, Pichia alcohol oxidase, alphavirus subgenomic promoters (such as Sindbis virus subgenomic promoters), antibiotic resistance gene, baculovirus, Heliothis zea insect virus, vaccinia virus, herpesvirus, raccoon poxvirus, other poxvirus, adenovirus, cytomegalovirus (such as intermediate early promoters), simian virus 40, retrovirus, actin, retroviral long terminal repeat, Rous sarcoma virus, heat shock, phosphate and nitrate transcription control sequences as well as other sequences capable of controlling gene expression in prokaryotic or eukaryotic cells.

### Host Cells

Also useful for certain embodiment of the invention is a recombinant cell comprising a host cell transformed with one or more recombinant molecules described herein or progeny cells thereof. Transformation of a polynucleotide molecule into a cell can be accomplished by any method by which a polynucleotide molecule can be inserted into the cell. Transformation techniques include, but are not limited to, transfection, electroporation, microinjection, lipofection, adsorption, and protoplast fusion. A recombinant cell may remain unicellular or may grow into a tissue, organ or a multicellular organism. Transformed polynucleotide molecules of the present invention can remain extrachromosomal or can integrate into one or more sites within a chromosome of the transformed (i.e., recombinant) cell in such a manner that their ability to be expressed is retained.

Suitable host cells to transform include any cell that can be transformed with a polynucleotide of the present invention. Host cells of the present invention either can be endogenously (i.e., naturally) capable of producing polypeptides described herein or can be capable of producing such polypeptides after being transformed with at least one polynucleotide molecule as described herein. Host cells of the present invention can be any cell capable of producing at least one protein defined herein, and include bacterial, fungal (including yeast), parasite, nematode, arthropod, animal and plant cells. Examples of host cells include Salmonella, Escherichia, Bacillus, Listeria, Saccharomyces, Spodoptera, Mycobacteria, Trichoplusia, BHK (baby hamster kidney) cells, CHO cells, 293 cells, EL4 cells, MDCK cells, CRFK cells, CV-1 cells, COS (e.g., COS-7) cells, and Vero cells. Further examples of host cells are E. coli, including E. coli K-12 derivatives; Salmonella typhi; Salmonella typhimurium, including attenuated strains; Spodoptera frugiperda; Trichoplusia ni; and non-tumorigenic mouse myoblast G8 cells (e.g., ATCC CRL 1246).

Recombinant DNA technologies can be used to improve expression of a transformed polynucleotide molecule by manipulating, for example, the number of copies of the polynucleotide molecule within a host cell, the efficiency with which those polynucleotide molecules are transcribed, the efficiency with which the resultant transcripts are translated, and the efficiency of post-translational modifications. Recombinant techniques useful for increasing the expression of polynucleotide molecules of the present invention include, but are not limited to, operatively linking polynucleotide molecules to high-copy number plasmids, integration of the polynucleotide molecule into one or more host cell chromosomes, addition of vector stability sequences to plasmids, substitutions or modifications of transcription control signals (e.g., promoters, operators, enhancers), substitutions or modifications of translational control signals (e.g., ribosome binding sites, Shine-Delgarno sequences), modification of polynucleotide molecules of the present invention to correspond to the codon usage of the host cell, and the deletion of sequences that destabilize transcripts.

### Gene Therapy

Therapeutic polynucleotide molecules described herein may be employed in accordance with the present invention by expression of such polynucleotides in treatment modalities often referred to as “gene therapy”. For example, polynucleotides encoding a compound of the invention, or a polynucleotide that up-regulates or down-regulates the production of a 5B6 ligand in a cell, may be employed in gene therapy, for example in the treatment of disease and/or modulating an immune response. Thus, cells from a patient may be engineered with a polynucleotide, such as a DNA or RNA, to encode a polypeptide ex vivo. The engineered cells can then be provided to a patient to be treated with the polynucleotide. In this embodiment, cells may be engineered ex vivo, for example, by the use of a retroviral plasmid vector containing RNA encoding a polypeptide described herein can be used to transform, for example, stem cells or differentiated stem cells. Such methods are well-known in the art and their use in the present invention will be apparent from the teachings herein.

Further, cells may be engineered in vivo for expression of a polypeptide in vivo by procedures known in the art. For example, a polynucleotide encoding a polypeptide as described herein may be engineered for expression in a replication defective retroviral vector or adenoviral vector or other vector (e.g., poxvirus vectors). The expression construct may then be isolated. A packaging cell is transduced with a plasmid vector containing RNA encoding a polypeptide as described herein such as a soluble fragment of human 5B6, such that the packaging cell now produces infectious viral particles containing the gene of interest. These producer cells may be administered to a patient for engineering cells in vivo and expression of the polypeptide in vivo. These and other methods for administering a polypeptide should be apparent to those skilled in the art from the teachings of the present invention.

Retroviruses from which the retroviral plasmid vectors hereinabove-mentioned may be derived include, but are not limited to, Moloney Murine Leukemia Virus, Spleen Necrosis Virus, Rous Sarcoma Virus, Harvey Sarcoma Virus, Avian Leukosis Virus, Gibbon Ape Leukemia Virus, Human Immunodeficiency Virus, Adenovirus, Myeloproliferative Sarcoma Virus, and Mammary Tumor Virus. In a preferred ° embodiment, the retroviral plasmid vector is derived from Moloney Murine Leukemia Virus.

Such vectors will include one or more promoters, for expressing the polypeptide. Suitable promoters which may be employed include, but are not limited to, the retroviral LTR; the SV40 promoter; and the human cytomegalovirus (CMV) promoter. Cellular promoters such as eukaryotic cellular promoters including, but not limited to, the histone, RNA polymerase III, the metallothionein promoter, heat shock promoters, the albumin promoter, the 5B6 promoter, human globin promoters and β-actin promoters, can also be used. Additional viral promoters which may be employed include, but are not limited to, adenovirus promoters, thymidine kinase (TK) promoters, and B19 parvovirus promoters. The selection of a suitable promoter will be apparent to those skilled in the art from the teachings contained herein.

The retroviral plasmid vector can be employed to transduce packaging cell lines to form producer cell lines. Examples of packaging cells which may be transfected include, but are not limited to, the PE501, PA317, Y-2, Y-AM, PA12, T19-14×, VT-19-17-H2, YCRE, YCRIP, GP+E-86, GP+envAm12, and DAN cell lines as described by Miller (1990). The vector may be transduced into the packaging cells through any means known in the art. Such means include, but are not limited to, electroporation, the use of liposomes, and CaPO₄ precipitation. In one alternative, the retroviral plasmid vector may be encapsulated into a liposome, or coupled to a lipid, and then administered to a host.

The producer cell line will generate infectious retroviral vector particles, which include the nucleic acid sequence(s) encoding the polypeptide (for example). Such retroviral vector particles may then be employed to transduce eukaryotic cells, either in vitro or in vivo. The transduced eukaryotic cells will express the nucleic acid sequence(s) encoding the polypeptide. Eukaryotic cells which may be transduced include, but are not limited to, embryonic stem cells, embryonic carcinoma cells, as well as hematopoietic stem cells, hepatocytes, fibroblasts, myoblasts, keratinocytes, myocytes (particularly skeletal muscle cells), endothelial cells, and bronchial epithelial cells.

Genetic therapies in accordance with the present invention may involve a transient (temporary) presence of the gene therapy polynucleotide in the patient or the permanent introduction of a polynucleotide into the patient.

Genetic therapies, like the direct administration of agents discussed herein, in accordance with the present invention may be used alone or in conjunction with other therapeutic modalities.

### Pharmaceutical Compositions, Dosages, and Routes of Administration

Compositions comprising the compound together with an acceptable carrier or diluent are useful in the methods of the present invention.

Therapeutic compositions can be prepared by mixing the desired component having the appropriate degree of purity with optional pharmaceutically acceptable carriers, excipients, or stabilizers (Remington's Pharmaceutical Sciences, 16th edition, Osol, A.ed. (1980)), in the form of lyophilized formulations, aqueous solutions or aqueous suspensions. Acceptable carriers, excipients, or stabilizers are preferably nontoxic to recipients at the dosages and concentrations employed, and include buffers such as Tris, HEPES, PIPES, phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; sugars such as sucrose, mannitol, trehalose or sorbitol; salt-forming counter-ions such as sodium; and/or non-ionic surfactants such as TWEEN™, PLURONICS™ or polyethylene glycol (PEG).

Additional examples of such carriers include ion exchangers, alumina, aluminum stearate, lecithin, serum proteins, such as human serum albumin, buffer substances such as glycine, sorbic acid, potassium sorbate, partial glyceride mixtures of saturated vegetable fatty acids, water, salts, or electrolytes such as protamine sulfate, disodium hydrogen phosphate, potassium hydrogen phosphate, sodium chloride, colloidal silica, magnesium trisilicate, polyvinyl pyrrolidone, and cellulose-based substances.

Therapeutic compositions to be used for in vivo administration should be sterile. This is readily accomplished by filtration through sterile filtration membranes, prior to or following lyophilization and reconstitution. The composition may be stored in lyophilized form or in solution if administered systemically. If in lyophilized form, it is typically formulated in combination with other ingredients for reconstitution with an appropriate diluent at the time for use. An example of a liquid formulation is a sterile, clear, colorless unreserved solution filled in a single-dose vial for subcutaneous injection.

Therapeutic compositions generally are placed into a container having a sterile access port, for example, an intravenous solution bag or vial having a stopper pierceable by a hypodermic injection needle. The compositions are preferably administered subcutaneously, intramuscularly or parenterally, for example, as intravenous injections or infusions or administered into a body cavity.

The compound may be administered in an amount of about 0.001 to 2000 mg/kg body weight per dose, and more preferably about 0.01 to 500 mg/kg body weight per dose. Repeated doses may be administered as prescribed by the treating physician.

Single or multiple administrations of the compositions are administered depending on the dosage and frequency as required and tolerated by the patient. The dosage and frequency will typically vary according to factors specific for each patient depending on the specific therapeutic or prophylactic agents administered, the severity and type of disease or immune response required, the route of administration, as well as age, body weight, response, and the past medical history of the patient. Suitable regimens can be selected by one skilled in the art by considering such factors and by following, for example, dosages reported in the literature and recommended in the Physician's Desk Reference, 56^{th} ed., (2002). Generally, the dose is sufficient to treat or ameliorate symptoms or signs of disease without producing unacceptable toxicity to the patient.

In another example, a compound useful for the methods of the invention comprises an antigen, such as a cancer or an antigen of a pathogen or infectious organism, and can be delivered by intramuscular, subcutaneous or intravenous injection, or orally, as a vaccine to enhance humoral and/or T cell mediated immune responses. In another example, the antigen is a self antigen or allergenic antigen which can used to diminish immune responses similar to that described for 33D1 and DEC-205 (Bonifaz et al., 2002; Finkelman et al., 1996).

In another example of the present invention, a radiolabeled form of the is delivered by intravenous injection as a therapeutic agent to target cells that express 5B6 or the 5B6 ligand. Previous examples of radiolabeled antibodies and the methods for their administration to patients as therapeutics are known to those skilled in the art. Examples include Iodine¹³¹ labeled Lym-1, against the β subunit of HLA-DR and the anti-CD20 Indium¹¹¹ and Yttrium⁹⁰ labeled Ibritumomab Tiuxetan (IDEC-Y2B8, ZEVALIN®) and Iodine I 131 Tositumomab (BEXXAR®).

In one embodiment, the composition does not comprise an adjuvant. In another embodiment, the composition does comprise an adjuvant. Examples of adjuvants include, but are not limited to, aluminium hydroxide, aluminium phosphate, aluminium potassium sulphate (alum), muramyl dipeptide, bacterial endotoxin, lipid X, polyribonucleotides, sodium alginate, lanolin, lysolecithin, vitamin A, saponin, liposomes, levamisole, DEAE-dextran, blocked copolymers or other synthetic adjuvants. Such adjuvants are available commercially from various sources, for example, Merck Adjuvant 65 (Merck and Company, Inc., Rahway, N.J.) or Freund's Incomplete Adjuvant and Complete Adjuvant (Difco Laboratories, Detroit, Mich.).

In an embodiment, the composition comprises liposomes or membrane vesicles. Examples of such liposomes are described in US 2007/0026057, Leserman (2004) and van Broekhoven et al. (2004). In these instances the compound can be used to target the liposome to enhance the delivery of an agent of interest. As outlined in US 2007/0026047, processes for the preparation of membrane vesicles for use in the invention are described in WO 00/64471.

Compositions for detection of cells with a disrupted cell membrane, cells infected with a pathogen, dying cells or dead cells, or a portion thereof, modulating an immune response, and/or antigen recognition, processing and/or presentation, are conventionally administered parenterally, by injection, for example, subcutaneously, intramuscularly or intravenously. Additional formulations which are suitable for other modes of administration include suppositories and, in some cases, oral formulations. For suppositories, traditional binders and carriers may include, for example, polyalkylene glycols or triglycerides; such suppositories may be formed from mixtures containing the active ingredient in the range of 0.5% to 10%, preferably 1% to 2%. Oral formulations include such normally employed excipients as, for example, pharmaceutical grades of mannitol, lactose, starch, magnesium stearate, sodium saccharine, cellulose, magnesium carbonate, and the like. These compositions take the form of solutions, suspensions, tablets, pills, capsules, sustained release formulations or powders and contain 10% to 95% of active ingredient, preferably 25% to 70%. Where the composition is lyophilised, the lyophilised material may be reconstituted prior to administration, e.g. as a suspension. Reconstitution is preferably effected in buffer. Capsules, tablets and pills for oral administration to a patient may be provided with an enteric coating comprising, for example, Eudragit “S”, Eudragit “L”, cellulose acetate, cellulose acetate phthalate or hydroxypropylmethyl cellulose.

In any treatment regimen, the therapeutic composition may be administered to a patient either singly or in a cocktail containing other therapeutic agents, compositions, or the like.

In an embodiment, the immune response is modulated by using a DNA vaccine encoding a compound of the invention conjugated to an antigen. DNA vaccination involves the direct in vivo introduction of DNA encoding the antigen into tissues of a subject for expression of the antigen by the cells of the subject's tissue. Such vaccines are termed herein “DNA vaccines” or “nucleic acid-based vaccines”. DNA vaccines are described in U.S. Pat. No. 5,939,400, U.S. Pat. No. 6,110,898, WO 95/20660, WO 93/19183, Demangel et al. (2005) and Nchinda et al. (2008).

To date, most DNA vaccines in mammalian systems have relied upon viral promoters derived from cytomegalovirus (CMV). These have had good efficiency in both muscle and skin inoculation in a number of mammalian species. A factor known to affect the immune response elicited by DNA immunization is the method of DNA delivery, for example, parenteral routes can yield low rates of gene transfer and produce considerable variability of gene expression. High-velocity inoculation of plasmids, using a gene-gun, enhanced the immune responses of mice, presumably because of a greater efficiency of DNA transfection and more effective antigen presentation by dendritic cells. Vectors containing the nucleic acid-based vaccine of the invention may also be introduced into the desired host by other methods known in the art, e.g., transfection, electroporation, microinjection, transduction, cell fusion, DEAE dextran, calcium phosphate precipitation, lipofection (lysosome fusion), or a DNA vector transporter.

Transgenic plants producing an antigenic polypeptide can be constructed using procedures well known in the art. A number of plant-derived edible vaccines are currently being developed for both animal and human pathogens. Immune responses have also resulted from oral immunization with transgenic plants producing virus-like particles (VLPs), or chimeric plant viruses displaying antigenic epitopes. It has been suggested that the particulate form of these VLPs or chimeric viruses may result in greater stability of the antigen in the stomach, effectively increasing the amount of antigen available for uptake in the gut.

### EXAMPLES

### Example 1

### Cloning and Expression of 5B6

### Materials and Methods

### Mice

Mice were bred under specific pathogen free conditions at The Walter and Eliza Hall Institute (WEHI). Female mice were used at 6-12 weeks of age; alternatively, gender aged-matched cohorts were generated. Animals were handled according to the guidelines of the National Health and Medical Research Council of Australia. Experimental procedures were approved by the Animal Ethics Committee, WEHI.

### Sequence Identification of 5B6

Sequencing was performed using the Big Dye Terminator version 3.1 (Applied Biosystems, Victoria, Australia) and 200 ng plasmid DNA, and subjected to electrophoresis on an ABI 3730x1 96-capillary automated DNA sequencer. Comparison of sequences to the expressed sequence tag, cDNA and protein databases was performed by basic local alignment search tool (BLAST) using National Center for Biotechnology Information (www.ncbi.nlm.nih.gov). Genomic localisation was performed by BLAT alignment to the mouse assembly (February 2006) and human assembly (March 2006) using University of California Santa Cruz, Genome Browser (vvww.genome.ucsc.edu/).

### Quantitative RT-PCR

RNA (up to 1 μg) was DNase treated with RQ1 DNase (Promega) then reverse transcribed into cDNA using random primers (Promega) and Superscript II reverse transcriptase (Gibco BRL, Geithersburg, Md.). Real-time reverse transcription PCR (RT-PCR) was performed to determine the expression of 5B6 and Gapdh in hemopoietic cells using the Quantitect SYBR Green PCR kit (Qiagen) and a Light cycler (Roche, Victoria; Australia). The specific primers for real-time RT-PCR were as follows: 5B6; 5′-TGTGACTGCTCCCACAACTGGA-3′ (SEQ ID NO:17); 5′-TTTGCACCAATCACAGCACAGA-3′ (SEQ ID NO:18), Gapdh; 5′-CATTTGCAGTGGCAAAGTGGAG-3′ (SEQ ID NO:19); 5′-GTCTCGCTCCTGGAAGATGGTG-3′ (SEQ ID NO:20). An initial activation step for 15 min at 95° C. was followed by 40 cycles of: 15s at 94° C. (denaturation), 20-30s at 50-60° C. (annealing) and 10-12s at 72° C. (extension), followed by melting point analysis. The expression level for each gene was determined using a standard curve prepared from 10⁻²-10⁻⁶ pg of specific DNA fragment, and was expressed as a ratio relative to Gapdh.

### Recombinant Surface Expression of 5B6

Full length mouse and human 5B6 (m5B6 and h5B6) were isolated by PCR amplification from splenic DC cDNA using Advantage cDNA polymerase (Clontech) and the following primers: [5B6: 5′-GCCATTTCTTGTACCAACCTACTCCT-3′(SEQ ID NO:21); 5′-CGGTGTGGTATGGATCGTCACTT-3′ (SEQ ID NO:22)], [HE: 5′-AGCCTCCTGTGTGGACTGCTTT-3′ (SEQ ID NO:23); 5′-TTCATGGCCCACATTTTGGTTT-3′ (SEQ ID NO:24)], and the resultant products were subcloned into pGemT easy plasmid (Promega). m5B6 and h5B6 were expressed on the surface of Chinese hamster ovary (CHO) cells as C-terminal (extracellular) FLAG-tagged proteins and on the surface of mouse EL4 cells as a fusion protein where green fluorescent protein (GFP) was fused to the N-terminal cytoplasmic domain of 5B6. To generate the FLAG tagged proteins, 5B6 encoding cDNA was amplified using Advantage high fidelity polymerase (Clontech), restriction digested with AscI and Mlu-1 and subcloned into a pEF-Bos vector modified to contain the FLAG epitope (kindly donated by Dr T. Willson; WEHI).

CHO cells were co-transfected with the pEF-Bos-5B6 lectin and a pGK-neo plasmid containing the neomycin phosphotransferase gene by electoporation (Gene Pulsar, Biorad, NSW, Australia) and transfectants selected with 1 mg/ml G418 (Geneticin, Life Technologies). 5B6 lectin-positive cells were stained with a rat anti-FLAG mAb, followed by an anti-rat Ig-PE (Caltag), and then isolated by flow cytometric sorting. GFP-tagged proteins were generated by amplifying the 5B6 lectin encoding cDNA, restriction digesting with EcoRI and subcloning into pEGFP-C2 vector (Clontech), before electroporation into EL4 cells and selection with 1 mg/ml G418. 5B6-positive cells were isolated by flow cytometric sorting of GFP positive cells. Full length untagged proteins were generated by amplifying the 5B6 lectin encoding cDNA, restriction digesting with EcoRI and subcloning into a pIRES-Neo vector, before electroporation into CHO cells and selection with 1 mg/ml G418.

### Generation of mAb Against C-type Lectins

Wistar rats were immunised three to four times with 50 μg Keyhole Limpet Hemocyanin (KLH)-conjugated peptide: 5B6 mouse peptide (H-DGSSPLSDLLPAERQRSAGQIC-OH) (SEQ ID NO:29), human peptide (H-RWLWQDGSSPSPGLLPAERSQSANQVC-OH) (SEQ ID NO:30), or 1×10⁷ CHO cells expressing 5B6-FLAG at 4 week intervals, and given a final boost 4 days before fusion with Sp2/0 myeloma cells. Hybridomas secreting specific mAb were identified by flow cytometric analysis of supernatants using CHO cells expressing C-type 5B6-FLAG and EL4 cells expressing GFP-5B6. Hybridomas were generated that displayed specific reactivity to each of mouse 5B6 and human 5B6.

In summary, the following mAb were previously generated and utilised in this study, two rat mAb 24/04-10B4 (from peptide immunisation) and 42/04-42D2 (from CHO-5B6-FLAG immunisation) were raised against mouse 5B6 (m5B6). Two rat antibodies 20/05-3A4 and 23/05-4C6 (from h5B6 peptide immunisation) were raised against h5B6 as described in WO 09/026,660.

### Isolation and Flow Cytometric Analysis of DCs

DC isolations from lymphoid organs were performed as previously described (Vremec et al., 2000). Briefly, tissues were mechanically chopped, digested with collagenase and DNAse and treated with ethylenediamine tetraacetic acid (EDTA). Low-density cells were enriched by density centrifugation (1.077 g/cm³ Nycodenz, Axis-Shield, Oslo, Norway). Non-DC-lineage cells were coated with mAb (KT3-1.1, anti-CD3; T24/31.7, anti-Thy1; TER119, anti-erythrocytes; ID3, anti-CD19; and 1A8, anti-Ly6G) then removed using anti-rat Ig magnetic beads (Biomag beads, QIAGEN, Victoria, Australia). Blood DCs were enriched by removing red blood cells (RBC) (0.168M NH₄Cl; 5 min at 4° C.) and depletion of irrelevant cells as above, except the mAb cocktail also contained the mAb F4/80. DC-enriched populations were blocked using rat Ig and anti-FcR mAb (2.4G2), then stained with fluorochrome-conjugated mAb against CD11c (N418), CD205 (NLDC-145), CD4 (GK1.5), CD8 (YTS169.4), CD24 (M1/69), 120G8 or CD45RA (14.8), Sirpα (p84) and m5B6 (24/04-10B4-biotin).

cDCs were selected as CD11c^{hi}CD45RA⁻ or CD11c^{hi}120G8⁻; splenic cDC were further subdivided into CD4⁺cDC (CD c^{hi}CD45RA⁻CD4⁺ CD8), double negative (DN) cDC (CD11c^{hi}CD45RA⁻CD4⁻CD8⁻) and CD8⁺cDC (CD11c^{hi}CD45RA⁻ CD8⁺ CD4⁻); thymic DCs were subdivided into CD8⁻cDC (Sirpα^{hi}CD8^{lo}) and CD8⁺cDC (Sirpα^{lo}CD8^{hi}); and LN cDC were subdivided into CD8⁻cDC (CD11c^{hi}CD205⁻CD8⁻), dermal DC (CD11c⁺CD205^{int}CD8⁻), Langerhans' cells (CD11c⁺CD205^{hi}CD8⁻) and CD8⁺cDC (CD11c⁺CD205^{hi}CD8⁺), as described previously (Lahoud et al., 2006). pDCs were separated as CD11c^{int}CD45RA⁺ or CD11c^{int}120G8⁺. Biotin staining was detected using streptavidin (SA)-phycoerythrin (PE). The expression of m5B6 on the various DC populations was analysed and compared to isotype control staining (IgG2a, BD Pharmingen, San Diego, Calif., USA). Flow cytometric analysis was performed on an LSR II (Becton Dickinson, Franklin Lakes, N.J., USA), excluding autofluorescent and propidium iodide (PI) positive dead cells.

### Isolation and Flow Cytometric Analysis of Human Blood DCs and Hemopoietic Cells

Peripheral blood mononuclear cells (PBMC) were isolated from human blood using Ficoll-Pacque-PLUS (GE Healthcare, Rydalmere, NSW, Australia) density separation. Blood donors gave with informed consent and collection was approved by Human Research Ethics Committee, Melbourne Health. The PBMC were blocked using rat Ig and anti-FcR mAb (2.4G2) then stained with mAb against HLA-DR (L243; Becton Dickinson), and a cocktail of PE-conjugated mAb against lineage markers, namely CD3 (BW264156; T cells), CD14 (Tuk4; monocytes), CD19 (6D5; B cells) and CD56 (AF12-7H3; NK cells). Blood DCs were gated as HLA-DR^{hi}, lineage⁻ cells and further segregated based on their expression of BDCA-1 (ADJ-8E3), BDCA-3 (AD5-14H2), BDCA-4 (AD5-17F6) and CD16 (VEP13). PBMC were also used as a source of other hemopoietic cells that were isolated using mAb against CD3 (BW264156; T cells), CD19 (6D5; B cells), CD56 (AF12-7H3), and NKp46 (9E2) (CD5.6⁺NKp46⁺; NK cells) and CD14 (Tuk4; monocytes). Staining and flow cytometric analysis for the expression of h5B6 (20/05-3A4) was performed, excluding PI positive dead cells. Unless otherwise specified, all anti-human mAb were purchased Miltenyi Biotec (North Ryde, NSW, Australia).

### Isolation and Analysis of 5B6 on Mouse Hemopoietic Cells

Spleen cell suspensions were prepared as for DC isolation (Vremec et al., 2000). Cells were stained with mAb against CD3 (KT3-1.1), CD19 (ID3), NK1.1 (PK136), CD49b (Hma2; eBioscience, San Diego, Calif., USA) then B cells (CD19⁺CD3⁻), T cells (CD19⁻CD3⁺) and NK cells (CD49b⁺NK1.1⁺CD3⁻) were selected. Splenic macrophages were first enriched by a 1.082 g/cm³ density centrifugation (Nycodenz) and immunomagnetic bead depletion of CD3⁺ T cells and CD19⁺ B cells; the enriched cells were stained with mAb against CD11b (M1/70) and F4/80, then macrophages were gated as CD11b^{hi}F4/80⁺. Bone marrow macrophages and monocytes were first enriched as for spleen, then stained with CD11b (M1/70) and Ly6C (5075-3.6); monocytes were then gated as side-scatter^{lo}Ly6C^{hi}CD11b^{hi} and macrophages as Ly6C^{int}CD11b^{hi}. All cells were blocked using rat Ig and anti-FcR mAb (2.4G2) before immunofluorescence staining with the various mAb cocktails including anti-5B6 mAb (10B4-biotin). Biotin staining was detected using streptavidin-PE. Samples were analysed for their expression of 5B6 on an LSR II (Becton Dickinson), excluding PI positive dead cells.

### Recombinant Expression of Soluble 5B6

To generate soluble 5B6, cDNA containing the hinge and ectodomain regions was amplified using Advantage high fidelity 2 polymerase (Clontech) and the following primers [m5B6: 5′-TAGTAGACGCGTGAGCAGCAGGAAAGACTCATC-3′ (SEQ ID NO:25); 5′-TAGTAGACGCGTTCAGATGCAGGATCCAAATGC-3′] (SEQ ID NO:26), [H₅B6:5′-TAGTAGACGCGTCAGCAGCAAGAAAAACTCATC-3′ (SEQ ID NO:27); 5′-TAGTAGACGCGTTCAGACAGAGGATCTCAACGC-3′] (SEQ ID NO:28). The amplified cDNA was restriction digested with Mlu-1 and subcloned into the Mlu-1 site of a pEF-Bos vector modified to contain the biotinylation consensus sequence (a peptide consensus sequence NSGLHHILDAQKMVWNHR (SEQ ID NO:31) recognised specifically by E coli biotin holoenzyme synthetase BirA and the FLAG epitope. The resulting lectin fusion constructs thus' included (in order of N-terminus): the IL3 signal sequence (to ensure secretion), the biotinylation consensus peptide sequence, a FLAG-tag, the hinge region and the lectin domain. Recombinant proteins were expressed by transient transfection of 293T cells (a human renal epithelial cell line stably transfected with polyoma/SV40 large T antigen) in DMEM-10% FCS with 8 micrograms DNA/75 cm2 flask using Fugene. After 8 h, the media was removed, the cells washed twice, then incubated for 36-60 h in 10 ml X-Vivo-10 protein-free/serum-free media (BioWhittaker, Walkersville, Md.). The media containing the secreted recombinant protein was harvested, and recombinant protein from the culture supernatant concentrated 100-fold using a 10,000 mwt cutoff centrifugal device (Nanosep 10K Omega, PALL Life Sciences). The concentrated protein was then used directly or enzymatically biotinylated using BIR enzyme (Avidity, Denver, Colo.).

### Binding Assays to Investigate the Binding of Soluble 5B6 to Membrane Bound 5B6

293T cells were transiently transfected with expression constructs encoding full length untagged 5B6 in pIRES Neo. Two-three days later, cells were harvested and surface immunofluorescence labeled using either (1) soluble FLAG-tagged biotinylated m5B6, h5B6 and Cire, and detected with Streptavidin PE, or (2) soluble FLAG-tagged 5B6, biotinylated anti-FLAG mAb 9H10, and Streptavidin-PE. Live cells were gated on forward and side scatter, or by propidium iodide exclusion and analysed for their surface binding of soluble 5B6. The specificity of the binding of soluble 5B6 was demonstrated by comparison to binding to other soluble FLAG-tagged C-type lectins, such as Cire.

### ELISA

Recombinant soluble protein secretion was assayed by capture/two-site ELISA. Briefly, 96-well polyvinylchloride microtitre plates (Costar, Broadway, Cambridge, UK) were coated with purified capture mAb, namely, anti-FLAG 9H10 12.5 ug/ml (generated in-house). Culture supernatants were detected using the biotinylated anti-m5B6 antibody (24/04-10B4)-(2 ug/ml), Streptavidin-HRP and ABTS substrate. Biotinylated recombinant soluble protein was assayed by capture/two-site ELISA. Briefly, 96-well polyvinylchloride microtitre plates (Costar, Broadway, Cambridge, UK) were coated with purified capture mAb, namely, anti-FLAG 9H10 12.5 ug/ml (generated in-house). Culture supernatants were detected using Streptavidin-HRP and ABTS substrate.

### Results

### Comparison of Gene Expression Patterns Between Splenic DC Subsets

Gene expression profile analysis identified a murine cDNA clone that is preferentially expressed by the CD8⁺ cDC subset relative to the CD8⁻ cDC. This clone, termed 5B6, represented a fragment of a “hypothetical C-type lectin”, a gene found on chromosome 6, that was differentially expressed in CD8⁺ DC (Riken 9830005G06, (recently named C-type lectin domain family 9, member A, (Clec9a) Genbank accession AK036399.1, Unigene ID Mm.391518). Furthermore, analysis of the public databases revealed a human orthologue for 5B6 (HEEE9341) on chromosome 12, recently renamed CLEC9A. Orthologs have been identified to exist in other animals such as chimpanzees (Genbank accession XP_{—}001143778), Rhesus monkeys (XP_{—}001114857), dogs (Genbank accession XP_{—}854151), cows (XP_{—}873119), horses (XP_{—}001493987) and rats (Genbank accession XP_{—}578403).

### Identification, Characterisation and Cloning of the C-Type Lectins

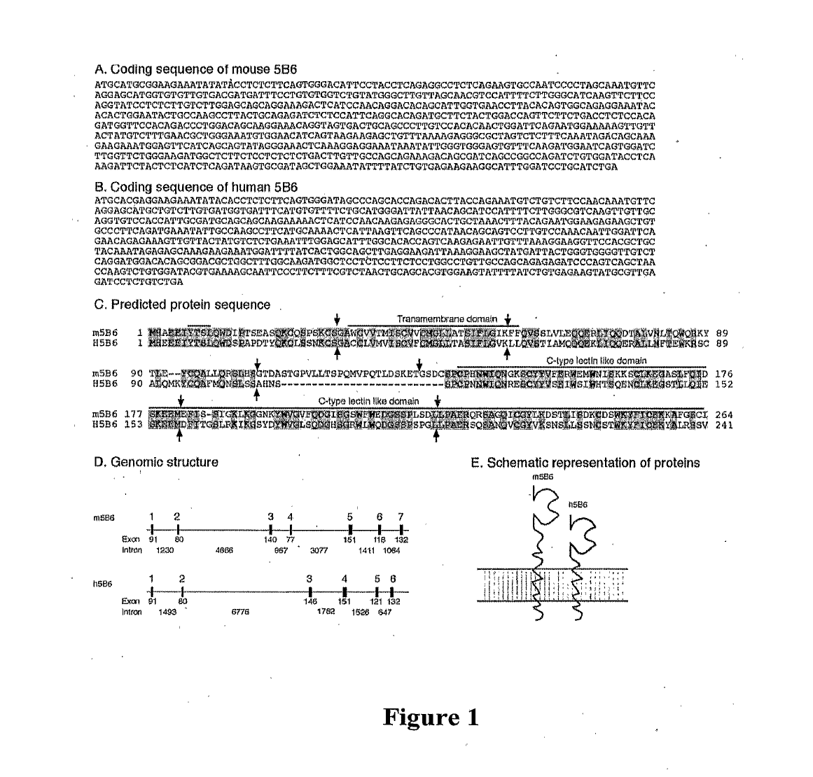

The inventors amplified the full-length cDNA encoding mouse and human 5B6 by PCR and sequenced the genes (FIGS. 1A and 1B).

The full-length coding sequence of mouse 5B6, encoded by 7 exons spanning 13.4 kb of genomic DNA (FIG. 1D), contains a single open reading frame (ORF) (795 bp) encoding a protein of 264 amino acid (aa) (FIG. 1C). Human 5B6 coding sequence, is encoded by 6 exons spanning 12.9 kb of genomic DNA (FIG. 1D), similarly contains a single ORF encoding a protein of 241 aa (FIG. 1C).

The mouse and human 5B6 gene each encode a putative transmembrane protein with a single C-type lectin domain in its extracellular region, a transmembrane region and a cytoplasmic tail containing the YXXL residues, which is a potential signalling motif (Fuller et al., 2007) (FIG. 1C). Human 5B6 has shorter hinge region than mouse. An alignment of the mouse and human protein sequences is demonstrated in FIG. 1C (53% identical; 69% similar). A schematic representation of the proposed mouse and human 5B6 protein structure is shown in FIG. 1E.

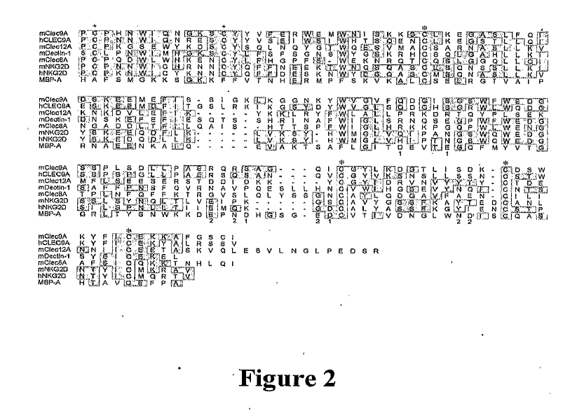

Using NCBI Blast protein analysis, it was determined that m5B6 shares most sequence similarity with mouse Dectin-1 (Clec7A), Clec12B, and NKG2D, whereas h5B6 is most similar to LOX-1 (Clec8A); Clec12B, and DCAL-2 (Clec12A). The CTLD of 5B6, like the classical C-type lectin the rat mannose binding protein A (MBP-A), has four conserved cysteine residues that form two disulfide bonds (FIG. 2). Furthermore, 5B6 possesses two additional cysteine residues in the neck region allowing protein homodimerization (Weis et al., 1998). Critically, the residues involved in Ca²⁺ binding in classical C-type lectins are not present in mouse and human 5B6 (FIG. 2).

### Expression of C-Type Lectin Genes

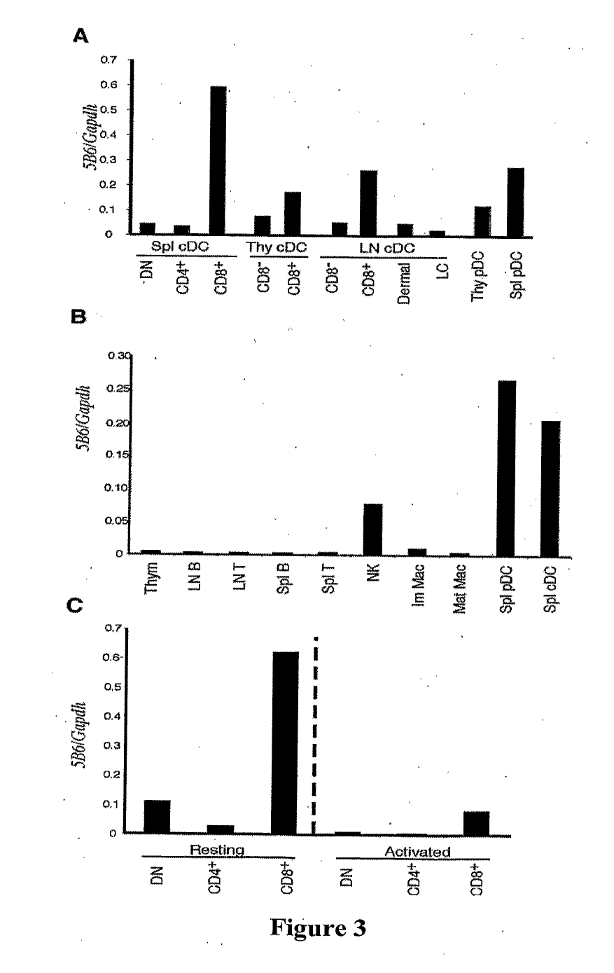

Microarray analysis predicted 5B6 to be expressed at 3.5 fold higher levels in CD8⁺ DC relative to CD8⁻ DC, and at 2.6-fold higher levels in CD8⁺ DC relative to the DN DC. Hence, the inventors designed primers and investigated the expression of 5B6, by quantitative RT-PCR, in mouse splenic cDC subsets. It was confirmed that 5B6 was preferentially expressed by the CD8⁺ cDC; splenic CD8⁺ DC expressed 22-fold more mRNA than splenic CD4⁺ cDC (FIG. 3A).

The inventors examined the expression of mouse and human 5B6 genes across a panel of hemopoietic cell types by quantitative real-time RT-PCR. 5B6. mRNA expression was specific to DC, both cDC and pDC, with moderate levels of mRNA expression in NK cells (FIG. 3B). It was preferentially expressed in splenic CD8⁺ DC relative to CD8⁻ cDC. It was also differentially expressed in the thymic CD8⁺ cDC and the LN CD8⁺DEC205^{hi} cDC (FIG. 3A). Furthermore, the gene expression in all three splenic cDC populations was reduced 3 h after in vivo activation with CpG and LPS, ligands to Toll like receptor 9 and 4 respectively (FIG. 3C).

### Surface Expression of Mouse 5B6 Protein

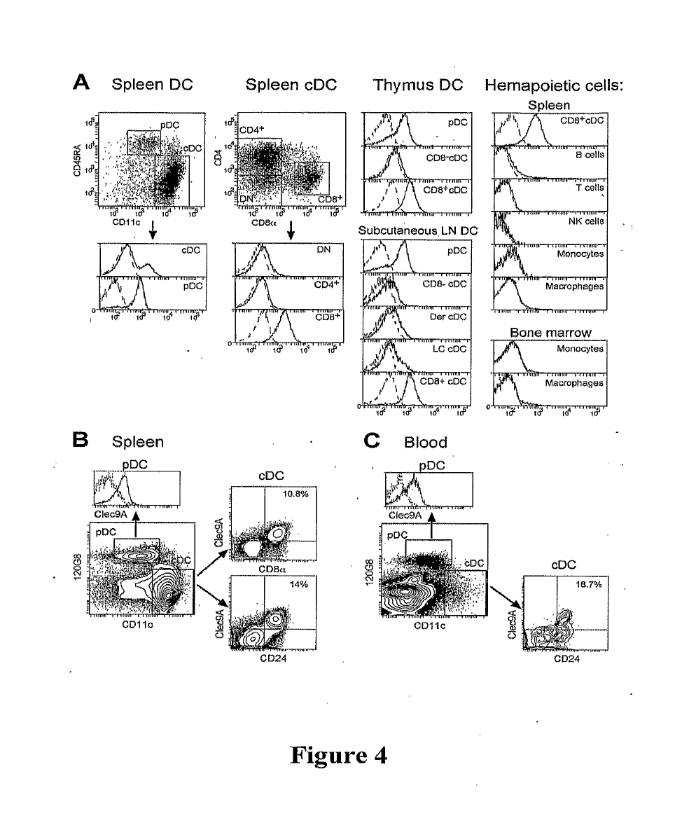

To investigate the protein expression of m5B6 and h5B6, we generated mAbs that recognised protein on the surface of 5B6-transfected cells by flow cytometry. Staining of a panel of freshly isolated mouse hemopoietic cells with the mAb 10B4 indicated that m5B6 was expressed on a subset of cDCs and on most pDCs (FIG. 4A). Strikingly, m5B6 protein was not detected on most other hemopoietic cells investigated, including T cells, most B cells, monocytes and macrophages. Nor was it detected on the NK cells that expressed some mRNA (FIG. 4A). However, a small (3%) proportion of B cells, displayed clear positive staining for m5B6. Only around 3% of bone marrow cells showed any staining with 10B4, and most of this was weak. Thus, in the hemopoietic system, m5B6 surface expression appears mainly restricted to DCs (FIG. 4A). In addition, staining of frozen sections with the mAb 10B4 revealed no staining beyond that attributed to DCs (data not shown).

Surface levels of m5B6 were then compared on splenic, LN and thymic cDCs. m5B6 was expressed by the CD8⁺cDCs of spleen, thymus and LN (FIG. 4A). Most splenic, thymic and LN CD8⁻cDCs and the migratory cDCs (dermal DCs and Langerhans' cells) were negative for m5B6 expression (FIG. 4A, B). However, a small proportion of CD8⁻cDCs showed above background staining; this could be attributed to a small proportion of DCs of the CD8⁺cDC lineage not yet expressing CD8α, known to be present within this CD8⁻cDC gating. No m5B6 staining was detected on a preparation of inflammatory CD11^{int}CD11b^{hi} DCs from inflamed mouse spleens (Naik et al., 2006) (data not shown). These DC surface expression profiles were consistent with the gene expression observed by quantitative RT-PCR (FIG. 3).

### Surface Expression of Mouse 5B6 on Mouse Blood DC

Mouse blood contains very few mature DC (CD11c^{hi}) compared to the DC found within the spleen and these few blood DC lack the expression of CD8 (O'Keeffe et al., 2003). However, in the mouse, CD24 expression has correlated with the expression of CD8. A small portion of mature DC within the blood express this marker; presumably these cells are on their way to becoming CD8⁺. To determine the expression of 5B6 on blood DC, we isolated them and stained them with CD24 and 5B6. DC expressing CD24 (which are destined to become CD8⁺DC) also express 5B6 (FIG. 4C).

### Surface Expression of Macaque and Human 5B6

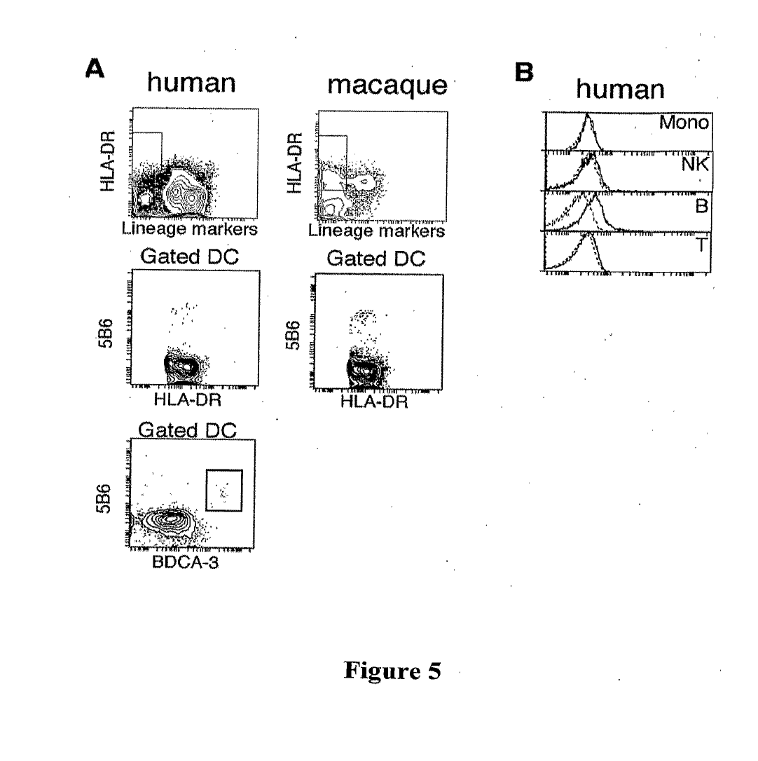

To investigate the surface expression of human 5B6 (h5B6), we generated two monoclonal antibodies (20/05-3A4; 23/05-4C6) that recognised native protein on the surface of h5B6-transfectant cells, as measured by flow cytometry (data not shown). Staining of freshly isolated peripheral blood cells, from humans or from macaque monkeys, indicated that 5B6 was expressed on a subset of DC (FIG. 5). In particular, a small subset of HLADR⁺ DCs were positive for h5B6 (FIG. 5A). Most other human blood cells did not show positive staining, but low level staining was obtained on human blood B cells (FIG. 5B). To determine if the 5B6-expressing DCs resembled those seen in mouse blood, the blood DCs were also stained with BDCA-1, BDCA-3 and BDCA-4. Staining with mAb 3A4 was restricted to the minor BDCA-3⁺ DC subset (proposed equivalents of mouse CD8⁺ cDC¹⁷), and absent from BDCA-4⁺ subset (data not shown). This suggests h5B6 is present on a cDC type similar to the mouse CD24⁺, CD8⁺ DC lineage (Galibert et al., 2005), but in contrast to the mouse, not on pDCs.

Soluble 5B6 can Interact with Membrane Bound 5B6 in a Cross-Species Manner

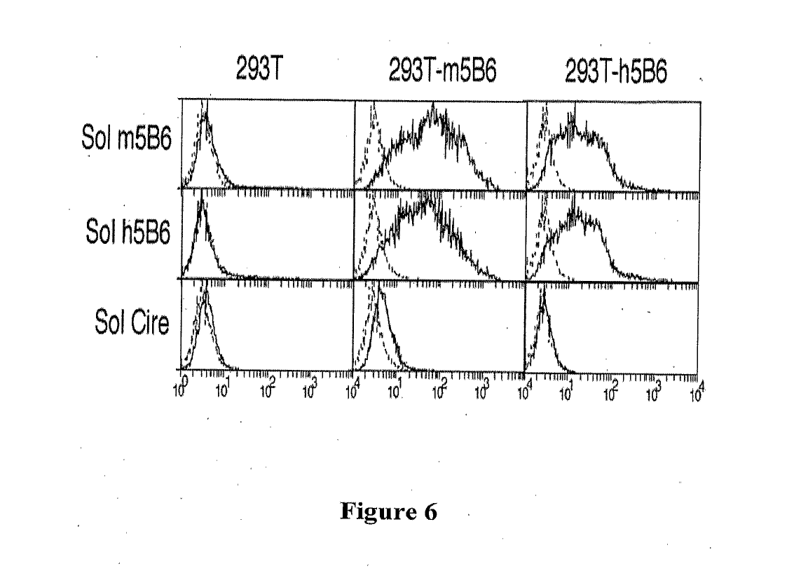

To identify binding partners for the 5B6 molecule, the inventors generated the soluble FLAG-tagged m5B6 and h5B6, and a control soluble FLAG-tagged C-type lectin Cire. The soluble 5B6 was screened for binding to 293T cells expressing membrane bound m5B6 and h5B6 following transient transfections with full length untagged 5B6 constructs in a pIresNeo vector. Soluble mouse 5B6 was able to bind to live 293T cells expressing both the membrane bound mouse 5B6 and human 5B6 but showed minimal or no binding to the mock (no DNA) transfected 293T cells (FIG. 6). Similarly, soluble human 5B6 was able to bind to live 293T cells expressing both the membrane bound mouse 5B6 and human 5B6 but showed no binding to mock transfected 293T cells. In contrast the control soluble molecule Cire showed only minimal binding to the control or transfectant cell lines. Thus, soluble 5B6 can interact with membrane bound 5B6 in a cross-species manner.

### Example 2

### 5B6 Ligand Expressed by Dying and Dead Cells, and Identification of 5B6 Ligands

### Materials and Methods

### Nomenclature

Throughout this Example 5B6 is referred to as Clec9A.

### Mice

Female C57BL/6J Wehi mice, 8-12 weeks of age, were bred under specific pathogen free conditions at The Walter and Eliza Hall Institute (WEHI); Animals were handled according to the guidelines of the National Health and Medical Research Council of Australia. Experimental procedures were approved by the Institutional Animal Ethics Committee, WEHI.

### Recombinant Expression of Soluble Clec9A

Two versions of soluble Clec9A were generated, a full Clec9A ectodomain (Clec9A-ecto; stalk and CTLD), and a Clec9A CTLD only (Clec9A-CTLD). Soluble ectodomain mouse Clec9A is provided as SEQ ID NO:40; soluble ectodomain human Clec9A is provided as SEQ ID NO:41, soluble CTLD only mouse Clec9A is provided as SEQ ID NO:42, and soluble CTLD only human Clec9A is provided as SEQ ID NO:43.

cDNA containing the required ectodomain region was amplified from the original Clec9A cDNA sequence (Caminschi et al., 2008) using Advantage high fidelity 2 polymerase (Clontech) or HotStar HiFidelity polymerase (Qiagen) and the following primers: mClec9A-ecto-forward: 5′-TAGTAGACGCGTGAGCAGCAGGAAAGACTCATC-3′ (SEQ ID NO:25); mClec9A-CTLD-forward: 5′-TAGTAGACGCGTGGTAGTGACTGCAGCCCTTGT-3′ (SEQ ID NO:38); mClec9A-reverse 5′-TAGTAGACGCGTTCAGATGCAGGATCCAAATGC-3′ (SEQ ID NO:26). hCLEC9A-ecto-forward: 5′-TAGTAGACGCGTCAGCAGCAAGAAAAACTCATC-3′ (SEQ ID NO:27); hCLEC9A-CTLD-forward: 5′-TAGTAGACGCGTAACAGCAGTCCTTGTCCAAACAAT-3′ (SEQ ID NO:39); hCLEC9A-reverse: 5′-TAGTAGACGCGTTCAGACAGAGGATCTCAACGC-3′ (SEQ ID NO:28).

The amplified cDNA was subcloned into a pEF-Bos vector modified to contain the biotinylation consensus sequence (a peptide consensus sequence NSGLHHILDAQKMVWNHR (SEQ ID NO:31) recognised specifically by E coli biotin holoenzyme synthetase BirA) and the FLAG epitope. The resulting fusion constructs thus included (in order of N-terminus): the IL3 signal sequence (to ensure secretion), the biotinylation consensus peptide sequence, a FLAG-tag, and Clec9A cDNA fragment. Tagged soluble ectodomain mouse Clec9A is provided as SEQ ID NO:44, tagged soluble ectodomain human Clec9A is provided as SEQ ID NO:45, tagged soluble CTLD only mouse Clec9A is provided as SEQ ID NO:46, and tagged soluble CTLD only human Clec9A is provided as SEQ ID NO:47.

Recombinant proteins were expressed by transient transfection of mammalian cells and culture in protein-free/serum-free media: 293T cells followed by culture in X-Vivo-10 media (BioWhittaker) or FreeStyle 293F cells cultured in FreeStyle Expression Media (Invitrogen). Media containing the secreted recombinant protein was assayed for the presence of soluble mClec9A by reactivity with anti-mouse Clec9A mAb (24/04-10B4). The secreted recombinant protein was concentrated 100-fold using a 10,000 mol wt cutoff centrifugal device (Millipore) and either used directly or enzymatically biotinylated using BIR enzyme (Avidity). Where required, Clec9A soluble proteins were purified by affinity chromatography using an anti-FLAG M2 agarose resin (Sigma) and elution with 100 μg/ml FLAG peptide (Auspep), and further purified by size-exclusion chromatography using a pre-packed Superdex 200 column (GE Healthcare). Furthermore, where required purified soluble Clec9A was specifically biotinylated using BIR enzyme (Avidity).

### Mouse Embryonic Fibroblasts

Mouse embryonic fibroblasts (MEF) expressing Noxa (van Delft et al., 2006) were seeded a day previously and grown to approximately 80% confluence then induced to undergo apoptosis by treatment with 2.5 μM ABT-737. After 16 h. cells were harvested using Cell Dissociation Buffer (Enzyme-free PBS-based; Gibco).

### Generation of Red Blood Cell Membranes

Mouse blood was collected into heparinised tubes, diluted 1:25 with PBS and harvested by centrifugation at 1204 g. Red blood cells (RBC) were enriched by collecting the heavy density fraction following density centrifugation (1.091 g/cm³ Nycondenz, Axis-Shield, Oslo, Norway), and washed a further 3 times with PBS before use. Purified RBC membranes were prepared by saponin lysis of RBC using saponin lysis buffer (0.15% Saponin (Sigma) in PBS, supplemented with a protease inhibitor cocktail (Roche)), centrifugation at 16.060 g and repeated washings with same buffer until the membrane cell pellet was white. RBC membranes were then used immediately for staining or frozen at −80° C. for purification of interacting proteins.

“Spectrin-free” membranes were then prepared using a modified protocol of Clana et al. (2005). In brief, RBC membranes were resuspended in 200 μl 5P8 (5 mM Na-phosphate, 0.5 mM EDTA, 0.2 mM PMSF, pH8.0) then treated with 10 ml 0.5 mM EDTA pH8.5, 0.33 mM DTT, 0.15 mM PMSF for 1 h at 37° C. “Spectrin-free” membranes were recovered by centrifugation at 27,000 g for 40 min at 4° C., and used immediately for staining or stored at −80° C.

### Binding Assays Using Soluble Clec9A

Binding assays were performed in binding buffer (PBS containing 0.2% BSA/and 0.02% sodium azide), on ice. Cells were washed 3 times with PBS to remove serum proteins, then resuspended in binding buffer. Cells were incubated with either (1) biotinylated soluble Clec9A and controls, and detected with SA-PE, or (2) soluble FLAG-tagged Clec9A, biotinylated anti-FLAG mAb 9H10, then detected with SA-PE. Live cells were gated on forward and side scatter, or by propidium iodide (PI) exclusion, whereas dead cells were gated on forward and side scatter, or by PI inclusion. Analysis of soluble Clec9A binding was performed by flow cytometry using a FACScan (Becton Dickinson). The specificity of the binding was demonstrated by comparison to binding to other soluble FLAG-tagged C-type lectins, mouse Cire/mDCSign (Caminschi et al., 2001) and Clec12A (Pyz et al., 2008).

### ELISA for the Detection of Clec9A Binding

ELISA plates (Costar, Broadway, Cambridge, UK) were coated overnight at 4° C. with 10 mg/ml of Spectrin (purified from human erythrocytes, Sigma, #S3644) or Actin (Sigma). Unbound proteins were washed away (PBS, 0.05% Tween-20), and the ELISA plates blocked with PBS, 1% BSA. Serially diluted biotinylated purified mClec9A-ecto, mClec9A-CTLD and Cire soluble proteins were plated (PBS, 1% BSA) and incubated at 4° C. overnight. Bound soluble proteins were detected using Streptavidin-HRP and visualised using ABTS.

### Identification of Interacting Proteins Using Protein Microarrays

Biotinylated purified soluble mClec9A-ecto and Cire (control) were diluted to 50 μg/ml in Casein Washing Buffer (Invitrogen) and hybridised to Human protein microarrays using the “ProtoArray Human Protein Microarray v4.1 Protein-Protein Interaction (PPI) kit for biotinylated proteins” (Invitrogen, #PAH05241011) as per manufacturer's instructions. Binding of the biotinylated proteins was detected using Streptavidin-Alexa Fluor647, and images acquired using a fluorescence microarray scanner (GenePix4000B scanner, Axon Instruments). Positive interactions were identified using the ProtoArray Prospector software (Invitrogen).

Phagocytic Uptake of Dead Cell by DCs from Lymphoid Organs

Splenic DC were isolated as previously described (Caminschi et al., 2008) and assayed for uptake of dead cells (modified from Schnorrer et al., 2006). In brief, DC were labelled with antibodies to CD11c (N418-allophycocyanin (APC)) and CD8 (YTS169.4-FITC). Splenocytes were subjected to two rounds of freezing then thawing (30s on dry ice followed by 30s at 37° C.) then labelled with 250 ng/ml PI for 10 min at 4° C., and the excess PI dye washed away. Labelled freeze-thawed splenocytes (1×10⁶ cells/well) were incubated with soluble proteins, mClec9A-ecto, mClec9A-CTLD or the control protein Cire, at 25 μg/ml for 30 min at 4° C., before the addition of 2.5×10⁵ DC in modified RPMI-1640 medium containing 10% FCS, 100 U/ml penicillin, 100 μg/ml streptomycin, and 10⁻⁴ M 2-ME. The cocultures were incubated for 3 h at 37° C. to enable phagocytosis, or at 4° C. (control) then harvested for flow cytometric analysis using an LSRII (Becton Dickinson). DC were gated as CD11c⁺CD8⁺ or CD11c⁺CD8⁻ cells and the proportion of DC that were PI⁺ calculated. Uptake at 4° C. was a measure of binding of dead splenocytes to the DC surface, whereas the additional uptake at 37° C. was a measure of phagocytic uptake.

### Results and Discussion

### Generation of Soluble Clec9A Ectodomains

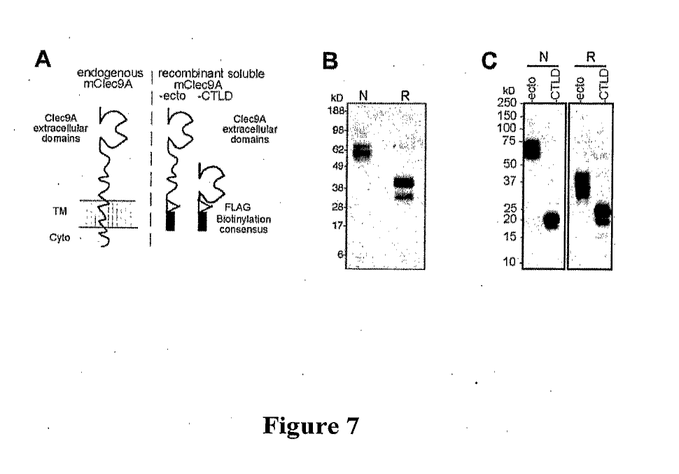

To seek Clec9A ligands, the inventors first generated tagged soluble forms of the ectodomains of both the mouse and human molecules. Constructs were made encoding either the full Clec9A ectodomain (Clec9A-ecto; stalk region and Clec9A CTLD), or the Clec9A CTLD only (Clec9A-CTLD), and including a FLAG-tag and a biotinylation consensus sequence (Brown et al., 1998) (FIG. 7A). Recombinant soluble mClec9A and hCLEC9A proteins were expressed in mammalian cells using protein-free/serum-free media. They were either enzymatically biotinylated using BirA (Avidity) enzyme (FIG. 7C) and detected with Streptavidin-PE (SA-PE), or they were detected using anti-FLAG reagents. As controls, similar constructs were generated for the other C-type lectins, mouse Cire/mDCSign (Caminschi et al., 2001) and Clec12A (Pyz et al., 2008).

### Mouse and Human Clec9A Bind to Dead Cells

A variety of mouse and human cells were screened to determine if soluble Clec9A could bind to the cell surface; low or minimal binding was observed with normal viable cells but we observed Clec9A binding to cells that were dead based on staining with the nuclear dye propidium iodide (PI) which stains cell nuclei once the cell membrane is damaged. Therefore, the inventors investigated the binding of mClec9A to thymocytes induced to undergo apoptosis using v-irradiation.

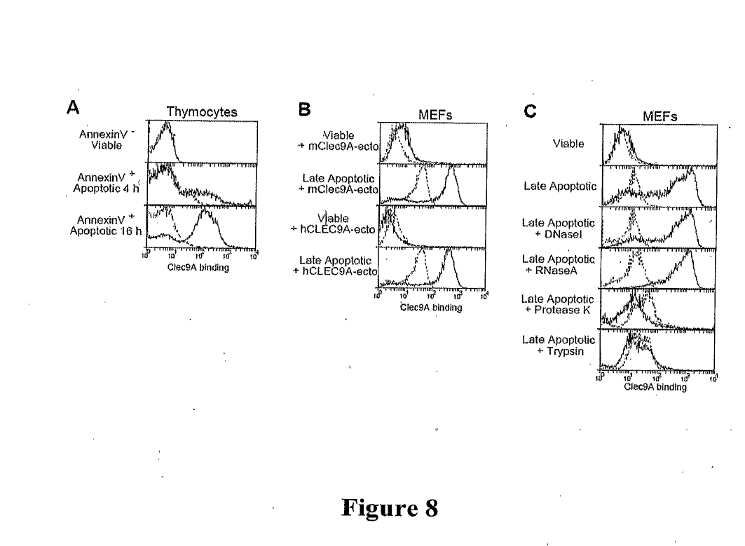

Thymocytes were stained with Annexin V, an early marker of apoptosis, and with PI to mark cells damaged to the point of having disrupted cell membranes mClec9A-ecto strongly bound to some apoptotic mouse thymocytes, but not to their viable counterparts; notably binding was restricted to late stage apoptotic/secondary necrotic cells (Annexin V⁺PI⁺) but not to early stage Annexin V⁺ apoptotic cells (FIG. 8A).

The present inventors further investigated mouse embryonic fibroblasts (MEF) induced to undergo apoptosis induced by the BH3 mimetic drug ABT-737. They found that both mClec9A and hCLEC9A strongly bound to late stage apoptotic MEFs, but not their live counterparts (FIG. 8B). Pretreatment of apoptotic cells with proteases (trypsin, protease K), but not with nucleases, reduced Clec9A binding in a dose dependent manner, suggesting the ligand was or ligands were a protein or protein-associated molecule(s) (FIG. 8C). The level of binding to dead cells was higher than any “non-specific” binding seen with soluble forms of other C-type lectins tested, namely Cire (FIG. 8A, B and C) and Clec12A (data not shown).

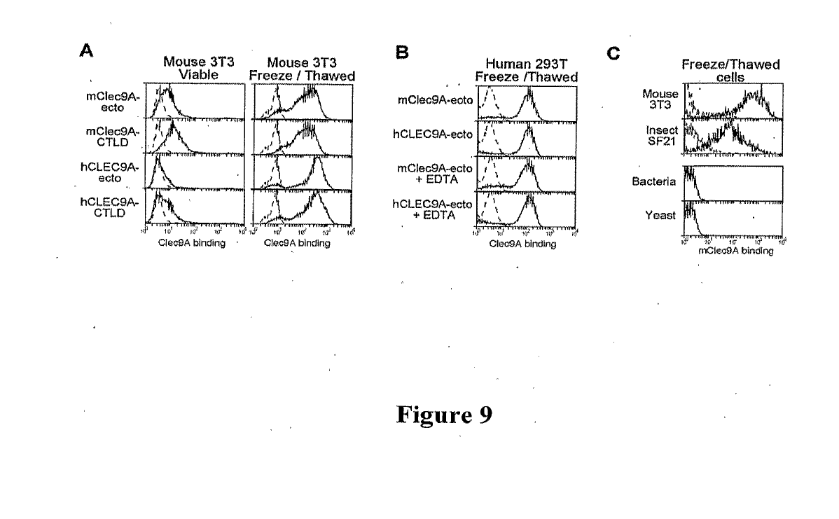

The possibility that cell membrane rupture was all that was required to reveal the ligand(s) was tested by staining with Clec9A immediately after freezing and thawing cells, as a model of primary necrosis. Clec9A bound to frozen and thawed 3T3 cells (FIG. 9A), to other frozen and thawed mouse cells, as well as to fixed and permeabilised cells and to frozen mouse tissue sections (data not shown). The binding included the exposed external surface of the dead cells, since it was also seen under fluorescence microscopy when fluorescent beads coated with mClec9A were used instead of soluble mClec9A (data not shown). Treatment of viable cells with trypsin prior to freeze-thawing did not eliminate Clec9A binding (data not shown), indicating the ligand(s) was initially within the cells.

In summary, the data indicates that the ligand(s) for Clec9A are normally contained within viable cells, and are only exposed after membrane disruption, such as occurs in late apoptosis or necrosis.

### Binding to Dead Cells is Via the C-Type Lectin Domain

To investigate the requirements for Clec9A binding to dead cells, the shorter form of the soluble recombinant proteins (CTLD only) was compared to the soluble full length ectodomains (stalk+CTLD). The mClec9A-CTLD and hCLEC9A-CTLD were both monomeric, compared to the homodimeric mClec9A ectodomains, indicating the stalk region was required for homodimerisation (FIG. 7). However, the mClec9A-CTLD and hCLEC9A-CTLD both showed similar levels of binding to dead cells as the full length ectodomains, indicating that the monomeric CTLD is sufficient for binding (FIG. 9A). The mClec9A-ecto and mClec9A-CTLD showed similar levels of binding even when limiting dilutions of Clec9A were used, indicating that the ectodomain and CTLD bound with similarly to the dead cells.

Since binding was unaffected by EDTA (FIG. 9B), divalent metal ions such as calcium are not required for binding.

### Species Conservation of Clec9A Binding to Dead Cells

As indicated in FIGS. 8 and 9A, both mouse and human Clec9A bound to dead mouse cells. Binding was seen to all dead mouse cell types tested, whether from cultured cell lines or freshly isolated cells. Mouse and human Clec9A also bound equally well to dead human cells (FIG. 9B), as well as to hamster and monkey cells (data not shown). Binding was also seen to frozen and thawed insect cells, but not to frozen and thawed bacteria or yeast (FIG. 9C). Thus, recognition of dead cells by Clec9A is conserved across evolution and the ligand(s) are expressed by most animal and even insect cells.

### Clec9A Binds to a Cytoskeletal Component of Cell Membranes

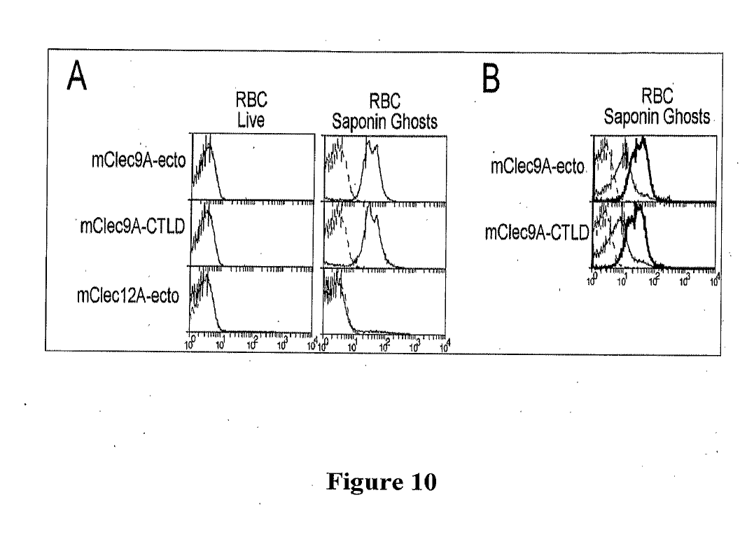

To determine whether Clec9A could bind to a ligand present within cell membranes, mClec9A was assayed for its ability to bind to membranes isolated from mouse red blood cells (RBC ghosts). RBC ghosts were prepared by lysis and repeated washing of the RBC in a saponin containing buffer, in order to permeabilise the cells and prevent resealing of the RBC membranes. Both Clec9A-ecto and Clec9A-CTLD bound to the RBC membranes, whereas two control proteins (Cire and Clec12A) showed no binding. Furthermore, Clec9A was found to bind at lower levels if the RBC membranes had been treated with a “spectrin-removal” buffer, indicating that Clec9A bound to spectrin or to a spectrin-associated cytoskeletal component (FIG. 10).

### Clec9A Binds to Purified Spectrin

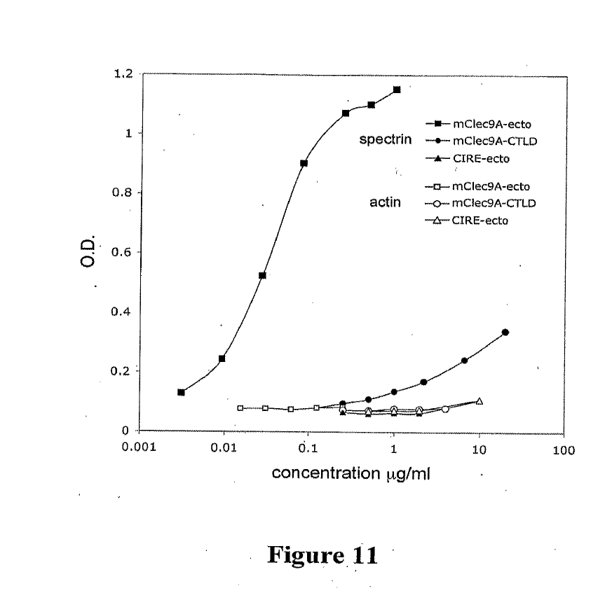

To determine whether Clec9A could interact directly with spectrin, soluble Clec9A-ecto, Clec9A-CTLD and the control protein Cire were assayed for their ability to bind to purified spectrin and to actin using an Enzyme Linked ImmunoSorbent Assay (ELISA). Purified biotinylated Clec9A soluble proteins (Clec9A-ecto and Clec9A-CTLD) bound to spectrin, whereas Cire did not bind to spectrin nor to actin (FIG. 11). mClec9A-ecto bound to spectrin more efficiently than Clec9A-CTLD, indicating that the stalk region of Clec9A may contribute to the (affinity of) interaction between Clec9A and spectrin (FIG. 11).

Clec9A-Ecto Interacts with RNF41

To determine whether Clec9A could interact with further molecules, purified biotinylated mClec9A-ecto and Cire were used to screen human protein microarrays. mClec9A-ecto was found to bind to isoforms of RNF41 (Accession number NM_{—}194358). Therefore RNF41 is another possible binding partner.

### Does Clec9A Mediate Uptake of Dead Cells by DC?

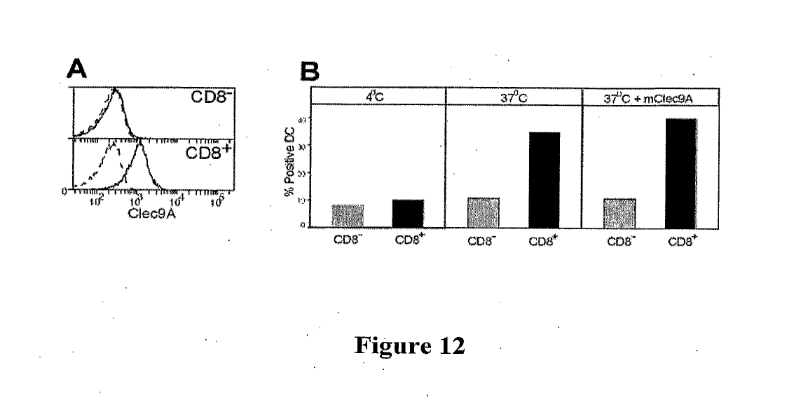

Clec9A is expressed on splenic DCs, and not on CD8−DCs, as reported previously (Caminschi et al., 2008; Sancho et al., 2008) and confirmed in FIG. 12A. CD8+ DCs have been reported previously to be more efficient at phagocytosis of dead cells (Iyoda et al., 2002; Schulz et al, 2002; Schnorrer et al., 2006). The inventors therefore investigated whether uptake of dead cells could be blocked using an excess of soluble Clec9A. As previously reported (Iyoda et al., 2002; Schulz and Sousa, 2002), the CD8+ DC were more efficient than their CD8− counterparts at phagocytosis of dead splenocytes that had been labelled with the nuclear dye PI (FIG. 12B). However, the addition of soluble mClec9A, whether the full ectodomain (mClec9A-ecto; FIG. 12B) or CTLD only (data not shown) had no noticeable effect on CD8+ DC uptake of dead cells. Similar results were observed using dead splenocytes that had been labelled with the lipophilic membrane dye PKH26 (data not shown).

### Conclusions

Clec9A binds strongly to late apoptotic or necrotic cells; it recognises a component or components that are expressed by cells of diverse origins and tissue types, but are not accessible until the cell membrane is disrupted. Clec9A can therefore serve to distinguish early apoptotic from necrotic cells. This is considered to be an important distinction in the biology of DCs, as uptake of early apoptotic cells has been reported to promote an immunosuppressive environment to self-Ag whereas necrosis or failed clearance of apoptotic cells has been reported to promote immunogenic responses. Clec9A is selectively expressed on CD8⁺ DCs, which are specialised for the uptake and processing of Ag from dead cells, so it is likely to have a role in this process. Spectrin and RNF41 both appear to bind to mClec9A, indicating these may constitute some of the binding partners for this molecule.

### Example 3

### Identification of the m5B6 Ligand

CD8+DC ingest dead cells more efficiently than other DC types (Iyoda et al., 2002). m5B6, expressed on CD8+ DC, specifically binds dead cells, but not early stage apoptotic cells. Molecules used by CD8+ DC to differentiate between early stage apoptotic cells and necrotic cells are of prime importance because uptake of early stage apoptotic cells by DC induces tolerance, but uptake of necrotic cells induces immunity (Sauter et al., 2000). Thus differential recognition of these states by receptors on DC is crucial to the immune system. Importantly, only CD8+ DC are capable of inducing efficient CD8 T cell responses to exogenous Ag (Belz et al., 2004).

Some related C-type lectins have had their ligands identified, and some have multiple ligands (eg. LOX-1/Clec8a, Dectin-1/Clec7a). The identity of 5B6 ligand(s) will be determined using a panel of immunochemical and proteomic techniques.

In a first approach, cells will be metabolically labelled using 35S, induce cell death, then incubate with soluble FLAG-tagged m5B6. Excess free Clec9A will be washed away and the cells incubated in the presence or absence of a chemical cross-linker. Cells will be lysed, and the complex affinity purified using either anti-FLAG M2 or anti-5B6-affinity resin. Bound proteins will be eluted with an excess of FLAG or 5B6 peptide.

In a complementary approach, a large batch of sol-5B6 will be purified and conjugated to NHS-activated Sepharose resin. Lysates from at least 5×10⁷ EL4 cells will be incubated with 5B6 affinity resin, and bound proteins eluted. Eluted proteins will be analysed by SDS-PAGE, transferred to PVDF membrane and visualised using a phosphorimager. To identify positive bands, this procedure will be scaled-up and eluates analysed by SDS-PAGE and Sypro Ruby/Coomassie blue staining. Bands will be excised and proteins identified using mass-spectrometry. In brief, protein bands will be digested with trypsin (Moritz et al., 1996), separated, by capillary chromatography (Moritz et al., 1992) and sequenced using an on-line electrospray ionisation ion-trap mass-spectrometer (Simpson et al., 2000).

In a further complementary approach, rats will be immunized with dead cells and perform a fusion to generate hybridomas by standard protocols. Hybridomas will be screened for Ab that bind to dead cells and block the binding of sol-5B6 as assayed by flow cytometry. We will clone and purify the blocking Ab, and use it to immunoprecipitate the ligand to enable identification by mass-spectrometry.

Myc-tagged expression constructs for potential ligands will be generated, transiently transfected into 293T cells and induce cell death, before incubation with 5B6+ DC or 5B6 transfectant cells. We will lyse the cells, immunoprecipitate 5136-complexes using anti-Clec9A mAb and analyse for co-precipitation of the myc-tagged ligand by Western blot. Alternatively, we will perform direct Western blots of 5B6 complexes. In a complementary approach, we will express potential candidates as recombinant soluble molecules and confirm their binding to 5B6 transfectants.

Antibodies which bind the 5B6 ligand will be generated using standard procedures.

### Example 4

### Clec9A binds RNF41

### Materials and Methods

### Expression and Purification of GST-RNF41 Proteins

cDNA constructs encoding two forms of RNF41 (full length RNF41 (SEQ ID NO:76); RNF41 transcript variant 2 (SEQ ID NO:77)) were subcloned into a modified pGEX-2T vector (GE Healthcare). Recombinant proteins were expressed as glutathione S-transferase (GST) fusion proteins in BL21 (DE3) E. coli and purified as described previously (Grieco et al, 1992). Briefly, BL21 (DE3) cells, transformed with plasmid, were cultured at 30° C. in Superbroth and induced when OD₆₀₀ reached ˜0.8 with 0.1 mM isopropyl-β-thiogalactopyranoside (IPTG) for 3 hours. The IPTG-induced cells were lysed with lysis buffer (0.2 mg/mL lysozyme, 1% Triton-X 100, 30 μg/mL DNase I, 1 mM PMSF in phosphate-buffered saline) for 1 hour on ice. The lysed cells were centrifuged at 16060 g for 15 minutes at 4° C. The resulting insoluble pellet containing the GST-RNF41 proteins was suspended initially in 1.5% N-lauroylsarcosine (Sarcosyl™, Sigma) for 10 min (4° C.) to extract GST-RNF41 proteins, and supplemented with 2% Triton X-100 and 1 mM CaCl₂ for an additional 10 min. The lysate was centrifuged (16060 g, 1.5 min). Supernatants containing the solubilised GST-RNF41 proteins were incubated with Glutathione-Sepharose 4B resin (GE Healthcare) for 1 h (4° C.). Glutathione-Sepharose resin coupled with GST-RNF41 fusion proteins was used in pull-down assays for detecting protein-protein interaction, as described below.

As a control, GST-protein was prepared by transforming BL21 cells with a plasmid control, IPTG induction, lysis and centrifugation. The resulting supernatant, containing the GST protein, was supplemented with 1.5% Sarcosyl, 1% Triton X-100, 1 mM CaCl₂. The GST protein was bound to Glutathione-Sepharose 4B resin and used as a control in the pull-down assays.

### Protein-Protein Interaction by the Pull-Down Assay

Glutathione Sepharose resin coupled with either GST or GST-RNF41 proteins was resuspended in the binding buffer (0.2% NP-40 and 2% glycerol in 20 mM Tris-buffered saline, pH 7.5 containing complete protease inhibitor cocktail mixture (Roche) and 1 mM PMSF) and mixed with 1 μg/m purified FLAG-tagged soluble Clec9A (also in binding buffer) and incubated at 4° C. on a rotating wheel for 2 hours. The beads were washed extensively with the binding buffer to remove unbound proteins. Bound proteins were eluted from the beads by the addition of 2×SDS reducing sample and heated at 92° C. for 5 minutes. The eluted proteins were separated by SDS-PAGE followed by Western blot with anti-FLAG antibody.

### Results

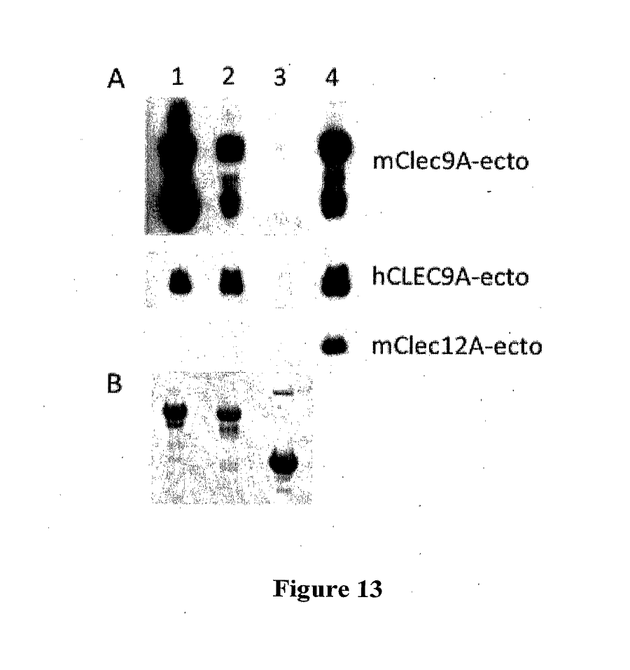

In an alternative approach to detect Clec9A interacting proteins, the inventors hybridised mClec9A-ecto and Cire, with protein microarrays (Invitrogen) consisting of glutathione S-transferase (GST)-tagged human proteins. mClec9A-ecto bound to an isoform of RNF41 (encoded by RNF41 transcript variant 2), whereas Cire-ecto did not bind RNF41. The inventors subcloned and expressed full length RNF41 (RNF41_{FL}), and RNF41 transcript variant 2 (RNF41₇₂₋₃₁₇) as GST-RNF41 fusion proteins in bacterial cells. The fusion proteins were immobilized onto glutathione beads, and incubated with mClec9A-ecto or with the control mClec12A-ecto. mClec9A-ecto directly bound to both GST-RNF41_{FL} and GST-RNF41₇₂₋₃₁₇, but not to GST alone (FIG. 13). In contrast, the control Clec 12A did not bind to RNF41. This confirmed that mClec9A binds specifically to RNF41. These studies were extended to investigate hCLEC9A interactions, and found that hClec9A-ecto similarly bound RNF41 (FIG. 13), indicating the Clec9A-RNF41 interaction is conserved among species.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

This application claims priority from U.S. 61/162,616 filed 23 Mar. 2009, the entire contents of which are incorporated herein by reference.

All publications discussed and/or referenced herein are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### REFERENCES

Almeida and Allshire (2005) TRENDS Cell Biol 15: 251-258.
Al-Mufti et al (1999) Am. J. Med. Genet. 85:66-75.
Bauer et al (2002) Int J Legal Med 116:39-42.
Belz et al (2004). Proc Natl Acad Sci USA 101:8670-8675.
Belz et al. (2005) J Immunol 175, 196-200 (2005).
Binder et al. (2004) Tissue Antigens 64, 442-51 (2004).
Bird et al (1988) Blood 80:1418-1422.
Bonifaz et al (2002) J Exp Med 196:1627-1638.
Bourque (1995) Plant Sci. 105:125-149.
Broderick and Winder (2005) Advan Prot Chem 70:203-246.
Brown et al (1998) J Exp Med 188, 2083-90.
Caminschi et al (2001) Mol Immunol 38:365-373.
Caminschi et al (2008) Blood 112:3264-3273.
Chothia and Lesk (1987) J. Mol. Biol. 196:901-917.
Ciana et al (2005) J Biosci 30:317-328.
Colcher et al (1986) Methods Enzymol. 121:802-816.
Delneste (2004) Biochem Soc Trans 32, 633-5.
Demangel et al (2005) Mol Immunol 42:979-985.
Dunbrack et al (1997) Folding and Design, 2:R27-42.
Finkelman et al (1996). J Immunol 157:1406-1414.
Fuller et al (2007) J Biol Chem 282:12397-12409.
Galibert et al (2005) J Biol Chem 280:21955-21964.
Greenwood et al (1993) Eur J Immunol 23: 1098-1104.
Grieco et al (1992) BioTechniques 13:856-857.
Harayama (1998) Trends Biotech. 16:76-82.
Haseloff and Gerlach (1988) Nature 334:585-591.
Heath and Valladangos (2005)
Henri et al (2001) J Immunol 167, 741-748 (2001).
Hochrein et al (2001) J Immunol 166:5448-5455.
Hoeffel et al (2007) Immunity 27, 481-92.
Hume (2008) Nat Immunol 9, 12-4.
Huston et al (1988) Proc Natl Acad Sci USA 85:5879-5883.
Iyoda et al (2002) J Exp Med 195, 1289-302.
Jones et al (1986) Nature 321:522-525.
Kenna et al (2008) Blood 111, 2091-100 (2008).
Lahoud et al (2006) J Immunol 177:372-382.
Leserman (2004) J Liposome Res 14:175-189.
Millar and Waterhouse (2005) Funct Integr Genomics 5:129-135.
Miller (1990) Blood 76:271-278.
Moritz et al. (1992) J Chromatogram 599:119-130.
Moritz et al. (1996) Electrophoresis 17:907-917.
Morrison et al (1984) Proc Natl Acad Sci USA 81:6851-6855.
Nagata (2007) Immunol Rev 220, 237-50 (2007).
Naik et al (2006) Nat Immunol 7:663-671.
Naik et al. (2005) J Immunol 174:6592-6597.
Nchinda et al (2008) J Clin Investig 118:1427-1436.
Needleman and Wunsch. (1970). J. Mol. Biol., 48, 443-453.
O'Keeffe et al (2002) J Exp Med 196:1307-1319.
O'Keeffe et al (2003) Blood 101:1453-1459.
Pasquinelli et al (2005) Curr Opin Genet Develop 15: 200-205.
Pastan et al (1986) Cell 47: 641-648.
Peng et al (2007) J Autoimmun 29:303-309.
Perriman et al (1992) Gene 113: 157-16.
Pyz et al (2008) Eur J Immunol 38:1157-1163.
Sancho et al (2008) J Clin Invest 118:2098-2110.
Sauter et al (2000) J Exp Med 191, 423-434.
Schnorrer et al (2006) Proc Natl Acad Sci USA 103:10729-10734.
Schulz & Sousa (2002) Immunology 107:183-189.
Senior (1998) Biotech. Genet. Engin. Revs. 15:79-119.
Shippy et al (1999) Mol. Biotech. 12: 117-129.
Shortman and Naik (2007). Nat Rev Immunol 7:19-30.
Simpson et al. (2000) Electrophoresis 21:1707-1732.
Smith et al (2000) Nature 407: 319-320.
Steinman et al (2000) J Exp Med 191, 411-6.
Steinman et al (2003) Annu Rev Immunol 21:685-711.
Sun et al (1986) Hybridoma 5S1:517-520.
Thorpe et al (1987) Cancer Res 47: 5924-31.
van Broekhoven et al (2004) Cancer Res 64:4357-4365.
van Delft et al (2006) Cancer Cell 10, 389-99
Vandenabeele et al (2001) Blood 97:1733-1741.
Vitetta et al (1987) Science 238: 1098-1104.
Vremec et al (2000) J Immunol 164:2978-2986.
Waldmann (1991) Science 252: 1657-1662.
Waterhouse et al (1998) Proc. Natl. Acad. Sci. USA 95: 13959-13964.
Weis et al (1998) Immunol Rev. 163:19-34.

1. A composition comprising compound comprising a polypeptide conjugate, wherein the polypeptide of the polypeptide conjugate comprises:
i) an amino acid sequence as provided in any one of SEQ ID NO's 48 to 80;
ii) an amino acid sequence which is at least 50% identical to any one or more of SEQ ID NO's 48 to 80; and/or
iii) a biologically active fragment of i) or ii),
and wherein the polypeptide of the compound binds a second polypeptide comprising
a) an amino acid sequence as provided in any one of SEQ ID NO's 1 to 8; and/or
b) an amino acid sequence which is at least 50% identical to any one or more of SEQ ID NO's 1 to 8.

2. The composition of claim 1, wherein the polypeptide comprises an amino acid sequence which is at least 90% identical to any one or more of SEQ ID NO's 48 to 80.

3. The composition of claim 1, wherein the polypeptide is conjugated to a therapeutic agent.

4. The composition of claim 3, wherein the therapeutic agent is an antigen.

5. The composition of claim 3, wherein the therapeutic agent is selected from the group consisting of a cytotoxic agent, a cancer antigen, a self antigen, an allergen, or an antigen from an infectious organism.

6. The composition of claim 3, wherein the therapeutic agent is a drug and/or pharmacological agent.

7. The composition of claim 1, wherein the polypeptide is conjugated to a detectable label.

8. A composition comprising a compound that binds a polypeptide which comprises:
i) an amino acid sequence as provided in any one of SEQ ID NO's 48 to 80;
ii) an amino acid sequence which is at least 50% identical to any one or more of SEQ ID NO's 48 to 80; and/or
iii) a biologically active fragment of i) or ii).

9. The composition of claim 8 which binds a polypeptide which comprises an amino acid sequence which is at least 90% identical to any one or more of SEQ ID NO's 48 to 80.

10. The composition of claim 8 which is a polypeptide.

11. The composition of claim 10 which is an antibody or antigenic binding fragment thereof.

12. The composition of claim 11, wherein the antibody is a monoclonal antibody, humanized antibody, single chain antibody, diabody, triabody, or tetrabody.

13. The compound according to claim 8 which is conjugated to a therapeutic agent or a detectable label.

14. The composition of claim 13, wherein the compound is conjugated to a therapeutic agent.

15. The composition of claim 14, wherein the therapeutic agent is an antigen.

16. The composition of claim 13, wherein the therapeutic agent is a cytotoxic agent, a cancer antigen, a self antigen, an allergen, and/or an antigen from an infectious organism.

17. The composition of claim 13, wherein the therapeutic agent is a drug and/or pharmacological agent.

18. (canceled)

19. The composition according to claim 1 wherein the composition comprises a pharmaceutically acceptable carrier.

20. The composition of claim 19 which comprises an adjuvant.

21. The composition of claim 19, wherein the compound is encapsulated in, or exposed on the surface of, a liposome.

22. A method of modulating an immune response in a subject, the method comprising administering to the subject a composition according to claim 1.

23. The method of claim 22, wherein an immune response to an antigen is induced and/or enhanced.

24. The method of claim 22, wherein an immune response to a self antigen or allergen is reduced.

25. A method of modulating an immune response to an antigen in a subject, the method comprising exposing dendritic cells or precursors thereof in vitro to a composition according to claim 3, and administering said cells to the subject.

26. The method of claim 25, wherein the cells have been isolated from the subject.

27.-28. (canceled)

29. A method of treating and/or preventing a disease involving dendritic cells or precursors thereof, the method comprising administering to the subject a composition according to claim 3, wherein.

30. (canceled)

31. A method of treating and/or preventing a disease involving dendritic cells or precursors thereof, the method comprising administering to the subject an isolated polynucleotide and/or construct encoding said polynucleotide which, when present in a cell of the subject, modulates the level of activity of a polypeptide which comprises:
i) an amino acid sequence as provided in any one of SEQ ID NO's 48 to 80; and/or
ii) an amino acid sequence which is at least 50% identical to any one or more of SEQ ID NO's 48 to 80,
in the cell when compared to a cell that lacks said polynucleotide.

32. The method of claim 31, wherein the polynucleotide down-regulates the level of activity of the polypeptide in the cell, and wherein the polynucleotide is selected from: an antisense polynucleotide, a sense polynucleotide, a catalytic polynucleotide, a microRNA, and a double stranded RNA.

33. The method according to claim 29, wherein the disease involving dendritic cells or precursors thereof is cancer, an infection, an autoimmune disease or an allergy.

34. The method of claim 33, wherein the autoimmune disease is lupus erythematosus.

35. The method of claim 33, wherein the infection is a Plasmodium sp. infection.

36. A method of modulating the uptake and/or clearance of cells with a disrupted cell membrane, cells infected with a pathogen, dying cells or dead cells, or a portion thereof, in a subject, the method comprising administering
i) a polypeptide comprising
a) an amino acid sequence as provided in any one of SEQ ID NO's 48 to 80;
b) an amino acid sequence which is at least 50% identical to any one or more of SEQ ID NO's 48 to 80; and/or
c) a biologically active fragment of a) or b),
ii) a composition according to claim 1.

37. A method of modulating the antigen recognition, processing and/or presentation of material derived from cells with a disrupted cell membrane, cells infected with a pathogen, dying cells or dead cells, or a portion thereof, or surrounding cells, in a subject, the method comprising administering
i) a polypeptide comprising
a) an amino acid sequence as provided in any one of SEQ ID NO's 48 to 80;
b) an amino acid sequence which is at least 50% identical to any one or more of SEQ ID NO's 48 to 80; and/or
c) a biologically active fragment of a) or b),
ii) a composition according to claim 1.

38. A method of modulating an immune response to material derived from cells with a disrupted cell membrane, cells infected with a pathogen, dying cells or dead cells, or a portion thereof, or surrounding cells, in a subject, the method comprising administering a
i) a polypeptide comprising
a) an amino acid sequence as provided in any one of SEQ ID NO's 48 to 80;
b) an amino acid sequence which is at least 50% identical to any one or more of SEQ ID NO's 48 to 80; and/or
c) a biologically active fragment of a) or b),
ii) a composition according to claim 1.

39. A method of modulating the uptake and/or clearance of cells with a disrupted cell membrane, cells infected with a pathogen, dying cells or dead cells, or a portion thereof, and/or modulating the antigen recognition, processing and/or presentation of material derived from cells with a disrupted cell membrane, cells infected with a pathogen, dying cells or dead cells, or a portion thereof, or surrounding cells and/or modulating an immune response to material derived from cells with a disrupted cell membrane, cells infected with a pathogen, dying cells or dead cells, or a portion thereof, or surrounding cells, in a subject, the method comprising:
administering to the subject a compound which modulates the production of a polypeptide which comprises:
i) an amino acid sequence as provided in any one of SEQ ID NO's 48 to 80;
ii) an amino acid sequence which is at least 50% identical to any one or more of SEQ ID NO's 48 to 80; and/or
iii) a biologically active fragment of i) or ii).

40.-41. (canceled)

42. The method according to claim 39, wherein the compound is a polynucleotide.

43. The method of claim 42, wherein the polynucleotide is operably linked to a promoter capable of directing expression of the polynucleotide in a cell of an animal.

44. The method of claim 42, wherein the polynucleotide down-regulates mRNA levels from a gene encoding the polypeptide.

45. The method of claim 44, wherein the polynucleotide is selected from: an antisense polynucleotide, a sense polynucleotide, a catalytic polynucleotide, a microRNA, and a double stranded RNA.

46. The method of claim 42, wherein the polynucleotide up-regulates mRNA levels from a gene encoding the polypeptide.

47. The method of claim 36, wherein the uptake and/or clearance of cells with a disrupted cell membrane, cells infected with a pathogen, dying cells or dead cells, or a portion thereof, is increased.

48. The method of claim 36, wherein the uptake and/or clearance of cells with a disrupted cell membrane, cells infected with a pathogen, dying cells or dead cells, or a portion thereof, is decreased.

49. The method of claim 37, wherein antigen recognition, processing and/or presentation of material derived from cells with a disrupted cell membrane, cells infected with a pathogen, dying cells or dead cells, or a portion thereof, or surrounding cells, is increased.

50. The method of claim 37, wherein antigen recognition, processing and/or presentation of material derived from cells with a disrupted cell membrane, cells infected with a pathogen, dying cells or dead cells, or a portion thereof, or surrounding cells, is decreased.

51. The method of claim 38, wherein the immune response to material derived from cells with a disrupted cell membrane, cells infected with a pathogen, dying cells or dead cells, or a portion thereof, or surrounding cells, is increased.

52. The method of claim 38, wherein the immune response to material derived from cells with a disrupted cell membrane, cells infected with a pathogen, dying cells or dead cells, or a portion thereof, or surrounding cells, is decreased.

53. The method according to claim 36, wherein the subject is suffering from a disease selected from: graft versus host disease (GVHD), an autoimmune disease, an infection, a neurodegenerative disease, systemic inflammatory reaction syndrome (SIRS), cancer and injury.

54.-62. (canceled)

63. A method of diagnosing, prognosing and/or monitoring the status of a disease associated with cells with a disrupted cell membrane, cells infected with a pathogen, dying cells or dead cells, the method comprising
i) contacting a cell with the composition of claim 8, and
ii) determining whether the polypeptide is present or absent,
wherein the presence of the polypeptide provides a diagnosis, prognosis and/or status of the disease.

64. The method of 63, wherein the compound is an antibody or antigenic binding fragment thereof.

65. The method of claim 63, wherein the compound is detectably labelled.

66. The method according to claim 63 which is performed in vivo on a subject.

67. The method according to claim 63 which is performed in vitro on a sample obtained from a subject.

68. The method according to claim 63, wherein the disease is selected from: graft versus host disease (GVHD), an autoimmune disease, an infection, a neurodegenerative disease, systemic inflammatory reaction syndrome (SIRS), cancer and injury.

69. A method of monitoring the effectiveness of a therapy for killing a cell, the method comprising;
i) exposing a cell to the therapy, and
ii) detecting a cell with a disrupted cell membrane, a dying cell or a dead cell, or a portion thereof, using a method according to claim 63,
wherein the presence of a cell with a disrupted cell membrane, a dying cell or a dead cell indicates that the therapy is effective.

70. The method of claim 69, wherein the cell in step i) is in vivo.

71. The method of claim 69, wherein the therapy is administered to a subject.

72. The method of claim 69, wherein the subject has cancer or an infection.

73. The method according to claim 69, wherein step ii) is performed on a sample obtained from a subject.

74. The method according to claim 69, wherein the therapy is drug therapy or radiotherapy.

75. A method of distinguishing between an early stage apoptotic cell and a late stage apoptotic cell, necrotic cell or dead cell, the method comprising
i) contacting a cell with a composition according to claim 1, and
ii) determining whether binding of the compound to the polypeptide is present or absent, wherein the compound binding to the polypeptide indicates that the cell is a late stage apoptotic cell, necrotic cell or dead cell.

76. A method of modulating an immune response to an antigen in a subject, the method comprising
i) obtaining a population of dendritic cells or precursors thereof,
ii) modulating the production and/or activity of a polypeptide which comprises:
a) an amino acid sequence as provided in any one of SEQ ID NO's 48 to 80;
b) an amino acid sequence which is at least 50% identical to any one or more of SEQ ID NO's 48 to 80; and/or
c) a biologically active fragment of a) or b),
iii) contacting the dendritic cells or precursors thereof with the antigen, and
iv) administering the dendritic cells or precursors thereof to the subject.

77. The method of claim 76, wherein step iii) comprises contacting the dendritic cells or precursors thereof with a cell with a disrupted cell membrane, a cell infected with a pathogen, a dying cell, a dead cell, and/or a portion thereof, comprising said antigen.

78. The method of claim 76, wherein step iii) comprises
a) obtaining a cell comprising the antigen,
b) disrupting the cell membrane of the cell, and
c) contacting the product of step b) with the dendritic cells or precursors thereof.

79. The method according to claim 76 which further comprises, before step ii), enriching the population for cells expressing the polypeptide.

80. A method of enriching dendritic cells, or a subset or precursors thereof, from a sample comprising;
i) contacting a sample comprising dendritic cells or precursors thereof with a compound according to claim 1, and
ii) isolating cells bound to the compound.

81. A method of enriching dendritic cells, or a subset or precursors thereof, from a sample comprising;
i) contacting a sample comprising dendritic cells or precursors thereof with a detectably labelled first polynucleotide that hybridizes to a second polynucleotide encoding a polypeptide which comprises
a) an amino acid sequence as provided in any one of SEQ ID NO's 48 to 80; and/or
b) an amino acid sequence which is at least 50% identical to any one or more of SEQ ID NO's 48 to 80, and
ii) isolating the detectably labelled cells.

82. The method of claim 80, wherein the cells obtained from step ii) are administered to a subject.

83. The method of claim 82, wherein the cells are administered to treat and/or prevent a disease selected from cancer, an infection, an autoimmune disease or an allergy.

84. A method of detecting dendritic cells, or a subset or precursors thereof, in a sample comprising;
i) contacting a sample comprising dendritic cells or precursors thereof with a compound according to claim 1, and
ii) detecting cells bound to the compound.

85. A method of detecting dendritic cells, or a subset or precursor thereof, in a sample comprising;
i) contacting a sample comprising dendritic cells or precursors thereof with a detectably labelled first polynucleotide that hybridizes to a second polynucleotide encoding a polypeptide which comprises
a) an amino acid sequence as provided in any one of SEQ ID NO's 48 to 80; and/or
b) an amino acid sequence which is at least 50% identical to any one or more of SEQ ID NO's 48 to 80, and
ii) detecting the detectably labelled cells.

86. A method of detecting dendritic cells, or a subset or precursor thereof, in a subject comprising;
i) administering to the subject a compound according to claim 1,
ii) detecting cells bound to the compound.

87. The method of claim 84, wherein the compound is detectably labelled.

88. A method of detecting dendritic cells, or a subset or precursor thereof, in a subject comprising;
i) administering to the subject a detectably labelled first polynucleotide that hybridizes to a second polynucleotide encoding a polypeptide which comprises
a) an amino acid sequence as provided in any one of SEQ ID NO's 48 to 80; and/or
b) an amino acid sequence which is at least 50% identical to any one or more of SEQ ID NO's 48 to 80, and,
ii) detecting the detectably labelled cells.

89. The method according to claim 80, wherein the dendritic cells express one or more of the following markers, CD8, CD24, Necl-2, CD11c, HLADR and BDCA3.

90. A method of detecting a cell with a disrupted cell membrane, a cell infected with a pathogen, a dying cell or a dead cell, the method comprising
i) contacting a cell with a compound that binds a polypeptide comprising
a) an amino acid sequence as provided in any one of SEQ ID NO's 48 to 80;
b) an amino acid sequence which is at least 50% identical to any one or more of SEQ ID NO's 48 to 80; and/or
c) a biologically active fragment of a) or b), and
ii) determining whether binding of the compound to the polypeptide is present or absent, wherein the compound binding to the polypeptide indicates that the cell has a disrupted cell membrane, is infected with a pathogen, is dying or is dead.

91. An isolated and/or exogenous polynucleotide encoding a compound according to claim 1, wherein the compound is a polypeptide.

92. An isolated polynucleotide which, when present in a cell of a subject, modulates the level of activity of a polypeptide in the cell when compared to a cell that lacks said polynucleotide, wherein the polypeptide comprises
i) an amino acid sequence as provided in any one of SEQ ID NO's 48 to 80;
ii) an amino acid sequence which is at least 50% identical to any one or more of SEQ ID NO's 48 to 80; and/or
iii) a biologically active fragment of i) or ii).

93. A vector comprising a polynucleotide of claim 91.

94. The vector of claim 93 which is an expression vector.

95. A host cell comprising at least one polynucleotide of claim 91.

96. The host cell of claim 95 which is a bacterial, yeast, insect, animal or plant cell.

97. An enriched population of dendritic cells and/or precursors thereof, obtained by a method according to any claim 80.

98. An expanded dendritic cell population, and/or precursors thereof, obtained by culturing an enriched population of dendritic cells and/or precursors thereof according to claim 97.

99. A composition comprising a polynucleotide of claim 91, and a pharmaceutically acceptable carrier.

100. A kit comprising a composition according to claim 1.
